SEQUENCE LISTING (114 sequences)

SEQ ID NO: 1 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 2 - protein, 133 aa, Artificial sequence [sequence omitted]

SEQ ID NO: 3 - protein, 525 aa, Homo sapiens
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 4 - protein, 154 aa, Artificial sequence
  synthetic sequence
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHFDPRDVVISNINVFVLELKGSETTFMCEYADETATIVEFLNRWITFCQSIISTLT

SEQ ID NO: 5 - protein, 134 aa, Artificial sequence
  synthetic sequence
APTSSSTKKTQLQLEHLLLDLQMILNGINNYKNPKLTRMLTFKFYMPKKATELKHLQCLEEELKPLEEVLNLAQSKNFHFDPRDVVISNINVFVLELKGSETTFMCEYADETATIVEFLNRWITFCQSIISTLT

SEQ ID NO: 6 - protein, 134 aa, Artificial sequence
  synthetic sequence
APTSSSTKKTQLQLEHLLLDLQMILNGINNYKNPKLTRMLTAKFYMPKKATELKHLQCLEEELKPLEEVLNLAQSKNFHFDPRDVVISNVNVFVLELKGSETTFMCEYADETATIVEFLNRWITFCQSIISTLT

SEQ ID NO: 7 - protein, 134 aa, Artificial sequence
  synthetic sequence
APTSSSTKKTQLQLEHLLLDLQMILNGINNYKNPKLTRMLTFKFYMPKKATELKHLQCLEEELKPLEEVLNLAQSKNFHFDPRDVVISNVNVFILELKGSETTFMCEYADETATIVEFLNRWITFCQSIISTLT

SEQ ID NO: 8 - protein, 134 aa, Artificial sequence
  synthetic sequence
APTSSSTKKTQLQLEHLLLDLQMILNGINNYKNPKLTRMLTFKFYMPKKATELKHLQCLEEELKPLEEVLNLAHSKNFHFDPRDVVISNINVFVLELKGSETTFMCEYADETATIVEFLNRWITFCQSIISTLT

SEQ ID NO: 9 - protein, 134 aa, Artificial sequence
  synthetic sequence
APTSSSTKKTQLQLEHLLLDLQMILNGINNYKNPKLTRMLTFKFYMPKKATELKHLQCLEEELKPLEEVLNLASSKNFHFDPRDVVISNINVFVLELKGSETTFMCEYADETATIVEFLNRWITFCQSIISTLT

SEQ ID NO: 10 - protein, 134 aa, Artificial sequence
  synthetic sequence
APTSSSTKKTQLQLEHLLLDLQMILNGINNYKNPKLTRMLTFKFYMPKKATELKHLQCLEEELKPLEEVLNLANSKNFHFDPRDVVISNINVFVLELKGSETTFMCEYADETATIVEFLNRWITFCQSIISTLT

SEQ ID NO: 11 - protein, 133 aa, Artificial sequence
  synthetic sequence
APTSSSTKKTQLQLSQLLVTLKLILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 12 - protein, 525 aa, Artificial sequence
  synthetic sequence
AVNGTSQFTCFYNSRANISCVWSQDGALQDTSCQVHAWPDRRRWNQTCELLPVSQASWACNLILGAPDSYKLDTVDIVTLRVLCREGVRWRVMAIQDFKPFENLRLMAPISLQVVHVETHRCNISWEISQASDFFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 13 - protein, 56 aa, Artificial sequence
  synthetic sequence
IPYKIEAVKSEPVEPPLPSQLHLMYVAAAAFVLLFFVGCGVLLSRKRRRQLCIQKL

SEQ ID NO: 14 - protein, 56 aa, Artificial sequence; identical to SEQ ID NO: 13
  synthetic sequence

SEQ ID NO: 15 - protein, 120 aa, Artificial sequence
  synthetic sequence
PPQIEEACELPECQVDAGNKVCNLQCNNHACGWDGGDCSLNFNDPWKNCTQSLQCWKYFSDGHCDSQCNSAGCLFDGFDCQLTEGQCNPLYDQYCKDHFSDGHCDQGCNSAECEWDGLDC

SEQ ID NO: 16 - protein, 152 aa, Artificial sequence
  synthetic sequence
AAGTLVLVVLLPPDQLRNNSFHFLRELSHVLHTNVVFKRDAQGQQMIFPYYGHEEELRKHPIKRSTVGWATSSLLPGTSGGRQRRELDPMDIRGSIVYLEIDNRQCVQSSSQCFQSATDVAAFLGALASLGSLNIPYKIEAVKSEPVEPPLP

SEQ ID NO: 17 - protein, 23 aa, Artificial sequence
  synthetic sequence
HLMYVAAAAFVLLFFVGCGVLLS

SEQ ID NO: 18 - protein, 306 aa, Artificial sequence
  synthetic sequence
PPQIEEACELPECQVDAGNKVCNLQCNNHACGWDGGDCSLNFNDPWKNCTQSLQCWKYFSDGHCDSQCNSAGCLFDGFDCQLTEGQCNPLYDQYCKDHFSDGHCDQGCNSAECEWDGLDCAEHVPERLAAGTLVLVVLLPPDQLRNNSFHFLRELSHVLHTNVVFKRDAQGQQMIFPYYGHEEELRKHPIKRSTVGWATSSLLPGTSGGRQRRELDPMDIRGSIVYLEIDNRQCVQSSSQCFQSATDVAAFLGALASLGSLNIPYKIEAVKSEPVEPPLPSQLHLMYVAAAAFVLLFFVGCGVLLS

SEQ ID NO: 19 - protein, 358 aa, Artificial sequence
  synthetic sequence
PCVGSNPCYNQGTCEPTSENPFYRCLCPAKFNGLLCHILDYSFTGGAGRDIPPPQIEEACELPECQVDAGNKVCNLQCNNHACGWDGGDCSLNFNDPWKNCTQSLQCWKYFSDGHCDSQCNSAGCLFDGFDCQLTEGQCNPLYDQYCKDHFSDGHCDQGCNSAECEWDGLDCAEHVPERLAAGTLVLVVLLPPDQLRNNSFHFLRELSHVLHTNVVFKRDAQGQQMIFPYYGHEEELRKHPIKRSTVGWATSSLLPGTSGGRQRRELDPMDIRGSIVYLEIDNRQCVQSSSQCFQSATDVAAFLGALASLGSLNIPYKIEAVKSEPVEPPLPSQLHLMYVAAAAFVLLFFVGCGVLLS

SEQ ID NO: 20 - protein, 37 aa, Artificial sequence
  synthetic sequence
PCVGSNPCYNQGTCEPTSENPFYRCLCPAKFNGLLCH

SEQ ID NO: 21 - protein, 37 aa, Artificial sequence
  synthetic sequence
DINECVLSPCRHGASCQNTHGGYRCHCQAGYSGRNCE

SEQ ID NO: 22 - protein, 37 aa, Artificial sequence
  synthetic sequence
DIDDCRPNPCHNGGSCTDGINTAFCDCLPGFRGTFCE

SEQ ID NO: 23 - protein, 37 aa, Artificial sequence
  synthetic sequence
DVNECDSQPCLHGGTCQDGCGSYRCTCPQGYTGPNCQ

SEQ ID NO: 24 - protein, 36 aa, Artificial sequence
  synthetic sequence
LVDECSPSPCQNGATCTDYLGGYSCKCVAGYHGVNC

SEQ ID NO: 25 - protein, 36 aa, Artificial sequence
  synthetic sequence
IDECLSHPCQNGGTCLDLPNTYKCSCPRGTQGVHCE

SEQ ID NO: 26 - protein, 27 aa, Artificial sequence
  synthetic sequence
CFNNGTCVDQVGGYSCTCPPGFVGERC

SEQ ID NO: 27 - protein, 10 aa, Artificial sequence
  synthetic sequence
GRRRRELDPM

SEQ ID NO: 28 - protein, 9 aa, Artificial sequence
  synthetic sequence
RQRRELDPM

SEQ ID NO: 29 - protein, 8 aa, Artificial sequence
  synthetic sequence
KIEAVKSE

SEQ ID NO: 30 - protein, 8 aa, Artificial sequence
  synthetic sequence
KIEAVQSE

SEQ ID NO: 31 - protein, 8 aa, Artificial sequence
  synthetic sequence
VGCGVLLS

SEQ ID NO: 32 - protein, 7 aa, Artificial sequence
  synthetic sequence
GCGVLLS

SEQ ID NO: 33 - protein, 248 aa, Artificial sequence
  synthetic sequence
MSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGGPADALDDFDLDMLPADALDDFDLDMLPADALDDFDLDMLPG

SEQ ID NO: 34 - protein, 211 aa, Artificial sequence
  synthetic sequence
MKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 35 - protein, 120 aa, Artificial sequence; identical to SEQ ID NO: 15
  synthetic sequence

SEQ ID NO: 36 - protein, 152 aa, Artificial sequence; identical to SEQ ID NO: 16
  synthetic sequence

SEQ ID NO: 37 - protein, 4 aa, Artificial sequence
  synthetic sequence
RQRR

SEQ ID NO: 38 - protein, 23 aa, Artificial sequence
  synthetic sequence
HLMYVAAAAFVLLFFVGCGVLLS

SEQ ID NO: 39 - protein, 4 aa, Artificial sequence
  synthetic sequence
VLLS

SEQ ID NO: 40 - protein, 37 aa, Artificial sequence; identical to SEQ ID NO: 20
  synthetic sequence

SEQ ID NO: 41 - protein, 56 aa, Artificial sequence; identical to SEQ ID NO: 13
  synthetic sequence

SEQ ID NO: 42 - protein, 206 aa, Artificial sequence
  synthetic sequence
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPG

SEQ ID NO: 43 - protein, 248 aa, Artificial sequence; identical to SEQ ID NO: 33
  synthetic sequence

SEQ ID NO: 44 - protein, 273 aa, Artificial sequence
  synthetic sequence
RPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWK

SEQ ID NO: 45 - protein, 242 aa, Artificial sequence
  synthetic sequence
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 46 - protein, 242 aa, Artificial sequence; identical to SEQ ID NO: 45
  synthetic sequence

SEQ ID NO: 47 - protein, 244 aa, Artificial sequence
  synthetic sequence
GSQVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGQGTTVTVSSGGGGSGGGGSSGGGSDIELTQSPAIMSASPGEKVTMTCSASSSVSYMHWYQQKSGTSPKRWIYDTSKLASGVPGRFSGSGSGNSYSLTISSVEAEDDATYYCQQWSKHPLTYGAGTKLEIKAS

SEQ ID NO: 48 - protein, 261 aa, Artificial sequence
  synthetic sequence
GSQVQLQQQVQLQESGGDLVKPGGSLKLSCAASGFTFSHYGMSWVRQTPDKRLEWVATIGSRGTYTHYPDSVKGRFTISRDNDKNALYLQMNSLKSEDTAMYYCARRSEFYYYGNTYYYSAMDYWGQGASVTVSSGGGGSGGGGSGGGGSDIVLTQSPAFLAVSLGQRATISCRASESVDNYGFSFMNWFQQKPGQPPKLLIYAISNRGSGVPARFSGSGSGTDFSLNIHPVEEDDPAMYFCQQTKEVPWTFGGGTKLEIK

SEQ ID NO: 49 - protein, 127 aa, Artificial sequence
  synthetic sequence
MADVQLVESGGGLVQAGGSLRLSCAASGRTFSTSAMGWFRQAPGKEREFVARITWSAGYTAYSDSVKGRFTISRDKAKNTVYLQMNSLKPEDTAVYYCASRSAGYSSSLTRREDYAYWGQGTQVTVS

SEQ ID NO: 50 - protein, 128 aa, Artificial sequence
  synthetic sequence
MADVQLVESGGGLVQAGGSLRLSCAASGRTISMAAMSWFRQAPGKEREFVAGISRSAGSAVHADSVKGRFTISRDNTKNTLYLQMNSLKAEDTAVYYCAVRTSGFFGSIPRTGTAFDYWGQGTQVTVS

SEQ ID NO: 51 - protein, 128 aa, Artificial sequence
  synthetic sequence
MAQVQLVESGGGLVQTGGSLKLSCTASVRTLSYYHVGWFRQAPGKEREFVAGIHRSGESTFYADSVKGRFTISRDNAKNTVHLQMNSLKPEDTAVYYCAQRVRGFFGPLRSTPSWYDYWGQGTQVTVS

SEQ ID NO: 52 - protein, 270 aa, Artificial sequence
  synthetic sequence
MAQVQLVESGGRLVQAGDSLRLSCAASGRTFSTSAMAWFRQAPGREREFVAAITWTVGNTILGDSVKGRFTISRDRAKNTVDLQMDNLEPEDTAVYYCSARSRGYVLSVLRSVDSYDYWGQGTQVTVSGGGGSGGGGSGGGGSMAQVQLVESGGGLVQAGGSLRLSCAASGRTFSNYAMGWFRQAPGKEREFVAAISWTGVSTYYADSVKGRFTISRDNDKNTVYVQMNSLIPEDTAIYYCAAVRARSFSDTYSRVNEYDYWGQGTQVTV

SEQ ID NO: 53 - protein, 242 aa, Artificial sequence; identical to SEQ ID NO: 45
  synthetic sequence

SEQ ID NO: 54 - protein, 211 aa, Artificial sequence; identical to SEQ ID NO: 34
  synthetic sequence

SEQ ID NO: 55 - protein, 205 aa, Artificial sequence
  synthetic sequence
LEIRAAFLRQRNTALRTEVAELEQEVQRLENEVSQYETRYGPLGGGKGGSGGSGGSMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAA

SEQ ID NO: 56 - protein, 185 aa, Artificial sequence
  synthetic sequence
GSQVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGQGTTVTVSSGGGGSGGGGSSGGGSDIELTQSPAIMSASPGEKVTMTCSASSSVSYMHWYQQKSGTSPKRWIYD

SEQ ID NO: 57 - protein, 115 aa, Artificial sequence
  synthetic sequence
PKKKRKVDALDDFDLDMLGSDALDDFDLDMLGSDALDDFDLDMLGSDALDDFDLDMLGSGGSGGSGGSLEIEAAFLERENTALETRVAELRQRVQRLRNRVSQYRTRYGPLGGGK

SEQ ID NO: 58 - protein, 198 aa, Artificial sequence
  synthetic sequence
PGLLGVSTLGPKRNSMVLDVAFVLEGSDKIGEADFNRSKEFMEEVIQRMDVGQDSIHVTVLQYSYMVTVEYPFSEAQSKGDILQRVREIRYQGGNRTNTGLALRYLSDHSFLVSQGDREQAPNLVYMVTGNPASDEIKRLPGDIQVVPIGVGPNANVQELERIGWPNAPILIQDFETLPREAPDLVLQRCCSGEGLQI

SEQ ID NO: 59 - protein, 198 aa, Artificial sequence
  synthetic sequence
PGLLGVKKLGPKRNSMVLDVAFVLEGSDKIGEADFNRSKEFMEEVIQRMDVGQDSIHVTVLQYSYMVTVEYPFSEAQSKGDILQRVREIRYQGGNRTNTGLALRYLSDHSFLVSQGDREQAPNLVYMVTGNPASDEIKRLPGDIQVVPIGVGPNANVQELERIGWPNAPILIQDFETLPREAPDLVLQRCCSGEGLQI

SEQ ID NO: 60 - protein, 199 aa, Artificial sequence
  synthetic sequence
PGIAGISSPGPKRKSMVLDVVFVLEGSDEVGEANFNKSKEFVEEVIQRMDVSPDATRISVLQYSYTVTMEYAFNGAQSKEEVLRHVREIRYQGGNRTNTGQALQYLSEHSFSPSQGDRVEAPNLVYMVTGNPASDEIKRLPGDIQVVPIGVGPHANMQELERISRPIAPIFIRDFETLPREAPDLVLQTCCSKEGLQLP

SEQ ID NO: 61 - protein, 199 aa, Artificial sequence
  synthetic sequence
PGIAGTLSPGPKRKSMVLDVVFVLEGSDEVGEANFNKSKEFVEEVIQRMDVSPDATRISVLQYSYTVTMEYAFNGAQSKEEVLRHVREIRYQGGNRTNTGQALQYLSEHSFSPSQGDRVEAPNLVYMVTGNPASDEIKRLPGDIQVVPIGVGPHANMQELERISRPIAPIFIRDFETLPREAPDLVLQTCCSKEGLQLP

SEQ ID NO: 62 - protein, 430 aa, Artificial sequence
  synthetic sequence
MVSKGEEDNSDVWWGGADTDYADGSEDKVVEVAEEEEVAEVEEEEADDDEDDEDGDEVEEEAEEPYEEATERTTSIATTTTTTTESVEEVVRVPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVNLDAEFRHDSGYEVHHQKLVFFAEDVGSNKGR

SEQ ID NO: 63 - protein, 194 aa, Artificial sequence
  synthetic sequence
GMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQVR

SEQ ID NO: 64 - protein, 461 aa, Artificial sequence
  synthetic sequence
PRHSKSHAAAQKQNNWIWSWFGNSQSTTQTQEPTTSATTALMTTPETPPKRQEAQNWFSWLFQPSESKSHLHTTTKMPGTESNTNPTGWEPNEENEDETDKYPSFSGSGIDDDEDFISSTIASTPRVSARTEDNQDWTQWKPNHSNPEVLLQTTTRMADIDRISTSAHGENWTPEPQPPFNNHEYQDEEETPHATSTTPNSTAEAAATQQETWFQNGWQGKNPPTPSEDSHVTEGTTASAHNNHPSQRITTQSQEDVSWTDFFDPISHPMGQGHQTESKDTDSSHSTTLQPTAAPNTHLVEDLNRTGPLSVTTPQSHSQNFSTLHGEPEEDENHPTTSILPSSTKSGAKDARRGGSLPTDTTTSVEGYTFQYPDTMENGTLFPVTPAKTEVFGETEVTLATDSNVNVDGSLPGDRDSSKDSRGSSRTVTHGSELAGHSSANQDSGVTTTSGPMRRPQIPER

SEQ ID NO: 65 - protein, 34 aa, Artificial sequence
  synthetic sequence
PRDEESYDSVTFDAHQYGATTQARADGLANAQVR

SEQ ID NO: 66 - protein, 163 aa, Artificial sequence
  synthetic sequence
AEMDREDAEHVKNSTYVALIIATDDGSPIATGTGTLLLVLLDVNDNAPIPEPRNMQFCQRNPQPHIITILDPDLPPNTSPFTAELTHGASVNWTIEYNDAAQESLILQPRKDLEIGEYKIHLKLADNQNKDQVTTLDVHVCDCEGTVNNCMKAGIVAAGLQVR

SEQ ID NO: 67 - protein, 342 aa, Artificial sequence
  synthetic sequence
NGAIFQETLSGAESTSLVAARGSCIANAEEVDVPIKLYCNGDGEWLVPIGRCMCKAGFEAVENGTVCRGCPSGTFKANQGDEACTHCPINSRTTSEGATNCVCRNGYYRADLDPLDMPCTTIPSAPQAVISSVNETSLMLEWTPPRDSGGREDLVYNIICKSCGSGRGACTRCGDNVQYAPRQLGLTEPRIYISDLLAHTQYTFEIQAVNGVTDQSPFSPQFASVNITTNQAAPSAVSIMHQVSRTVDSITLSWSQPDQPNGVILDYELQYYEKQELSEYNATAIKSPTNTVTVQGLKAGAIYVFQVRARTVAGYGRYSGKMYFQTMTEAEYQTSIKEKLPR

SEQ ID NO: 68 - protein, 110 aa, Artificial sequence
  synthetic sequence
APSAVSIMHQVSRTVDSITLSWSQPDQPNGVILDYELQYYEKQELSEYNATAIKSPTNTVTVQGLKAGAIYVFQVRARTVAGYGRYSGKMYFQTMTEAEYQTSIKEKLPR

SEQ ID NO: 69 - protein, 233 aa, Artificial sequence
  synthetic sequence
MAMARSRRDSVWKYCWGLLMVLCRTAISRSIVLEPIYWNSSNSKFLPGQGLVLYPQIGDKLDIICPKVDSKTVGQYEYYKVYMVDKDQADRCTIKKENTPLLNCARPDQDVKFTIKFQEFSPNLWGLEFQKNKDYYIISTSNGSLEGLDNQEGGVCQTRAMKILMKVGQDASSAGSARNHGPTRRPELEAGTNGRSSTTSPFVKPNPGSSTDGNSAGHSGNNLLGSEVALFAR

SEQ ID NO: 70 - protein, 47 aa, Artificial sequence
  synthetic sequence
VYVRPTNETLYEAPEPIFTSNSSCSGLGGCHLFLTTVPVLWSLLGSR

SEQ ID NO: 71 - protein, 66 aa, Artificial sequence
  synthetic sequence
GQDASSAGSARNHGPTRRPELEAGTNGRSSTTSPFVKPNPGSSTDGNSAGHSGNNLLGSEVALFAR

SEQ ID NO: 72 - protein, 211 aa, Artificial sequence
  synthetic sequence
DNQQMRERRAVSNFSTASQIYEAPKMLSMLFRTYKDQGQILYAATNQMFTSLSLREGRLVYYSKQHLTINMTVQETSTLNDGKWHNVSLFSESRSLRLIVDGRQVGDELDIAGVHDFLDPYLTILNVGGEAFVGCLANVTVNNELQPLNGSGSIFPEVRYHGKIESGCRGDIGQDAAQVADPLSIGFTLVIVFFVILVVAILGSYVIYRFR

SEQ ID NO: 73 - protein, 370 aa, Artificial sequence
  synthetic sequence
MACKMQIFVKTLTGKTITLEVEPSDTIENVKAKIQDKEGIPPDQQRLIFAGKQLEDGRTLSDYNIQKESTLHLVLRLRGGELGGSGGSGEGRVKESITRRRRAPSVANVGSHSDLSLKIPEISIQDMTAQVTSPSGKTHEAEIVEGENHTYSIRFVPAEMGTHTVSVKYKGQHVPGSPFQFTVGPLGEGGAHKVRAGGPGLERAEAGVPAEFSIWTREAGAGGLAIAVEGPSKAEISFEDRKDGSSGVAYVVQEPGDYEVSVKFNEEHIPDSPFVVPVASPSSGGSGGTMQIFVKTLTGKTITLEVEPSDTIENVKAKIQDKEGIPPDQQRLIFAGKQLEDGRTLSDYNIQKESTLHLVLRLRGGKCLER

SEQ ID NO: 74 - protein, 293 aa, Artificial sequence
  synthetic sequence
MACKMQIFVKTLTGKTITLEVEPSDTIENVKAKIQDKEGIPPDQQRLIFAGKQLEDGRTLSDYNIQKESTLHLVLRLRGGELGGSGGPTFRSSLFLWVRPGGSGGSGPLGEGGAHKVRAGGPGLERAEAGVPAEFSIWTREAGAGGLAIAVEGPSKAEISFEDRKDGSCGVAYVVQEPGDYEVSVKFNEEHIPDSPFVVPVASPSSGGSGGTMQIFVKTLTGKTITLEVEPSDTIENVKAKIQDKEGIPPDQQRLIFAGKQLEDGRTLSDYNIQKESTLHLVLRLRGGKCLER

SEQ ID NO: 75 - protein, 515 aa, Artificial sequence
  synthetic sequence
MVSKGEETTMGVIKPDMKIKLKMEGNVNGHAFVIEGEGEGKPYDGTNTINLEVKEGAPLPFSYDILTTAFAYGNRAFTKYPDDIPNYFKQSFPEGYSWERTMTFEDKGIVKVKSDISMEEDSFIYEIHLKGENFPPNGPVMQKKTTGWDASTERMYVRDGVLKGDVKHKLLLEGGGHHRVDFKTIYRAKKAVKLPDYHFVDHRIEILNHDKDYNKVTVYESAVARNSTDGMDELYKGPGGAGPGGAGPGGAGPGGAGPGGAGPGGAGPGGAGPGGAMVSKGEELFTGVVPILVELDGDVNGHKFSVSGEGEGDATYGKLTLKLICTTGKLPVPWPTLVTTLGYGLQCFARYPDHMKQHDFFKSAMPEGYVQERTIFFKDDGNYKTRAEVKFEGDTLVNRIELKGIDFKEDGNILGHKLEYNYNSHNVYITADKQKNGIKANFKIRHNIEDGGVQLADHYQQNTPIGDGPVLLPDNHYLSYQSKLSKDPNEKRDHMVLLEFVTAAGITLGMDELYK

SEQ ID NO: 76 - protein, 328 aa, Artificial sequence
  synthetic sequence
AARSCRFRGRHYKREFRLEGEPVALRCPQVPYWLWASVSPRINLTWHKNDSARTVPGEEETRMWAQDGALWLLPALQEDSGTYVCTTRNASYCDKMSIELRVFENTDAFLPFISYPQILTLSTSGVLVCPDLSEFTRDKTDVKIQWYKDSLLLDKDNEKFLSVRGTTHLLVHDVALEDAGYYRCVLTFAHEGQQYNITRSIELRIKKKKEETIPVIISPLKTISASLGSRLTIPCKVFLGTGTPLTTMLWWTANDTHIESAYPGGRVTEGPRQEYSENNENYIEVPLIFDPVTREDLHMDFKCVVHNTLSFQTLRTTVKEASSTFSGR

SEQ ID NO: 77 - protein, 232 aa, Artificial sequence
  synthetic sequence
KRPKPGGWNTGGSRYPGQGSPGGNRYPPQGGTWGQPHGGGWGQPHGGSWGQPHGGSWGQPHGGGWGQGGGTHNQWNKPSKPKTNLKHVAGAAAAGAVVGGLGGYMLGSAMSRPMIHFGNDWEDRYYRENMYRYPNQVYYRPVDQYSNQNNFVHDCVNITIKQHTVTTTTKGENFTETDVKMMERVVEQMCVTQYQKESQAYYDGRRSSSTVLFSSPPVILLISFLIFLIVGR

SEQ ID NO: 78 - protein, 41 aa, Artificial sequence
  synthetic sequence
GDRCQNYVMASFYKHLGIEFMEAEELYQKRVLTITGICIAR

SEQ ID NO: 79 - protein, 73 aa, Artificial sequence
  synthetic sequence
PRNPQCVRWNSFTNRWTRLGCQTEIPDFDGDFNPAAQQAILVNCSCTHISSYAVIVDVIDPEDIPEPSLLVQR

SEQ ID NO: 80 - protein, 116 aa, Artificial sequence
  synthetic sequence
ITYPSEQMQQSEQVVYRSLGSPHLAQPIKLQMWLDVDSARFGPRSNPQCVRWNSFTNRWTRLGCQTEIPDFDGDFNPAAQQAILVNCSCTHISSYAVIVDVIDPEDIPEPSLLVQR

SEQ ID NO: 81 - protein, 44 aa, Artificial sequence
  synthetic sequence
PRGPGGAGPGGAGPGGAGPGGAGPGGAGPGGAGPGGAGPGGARR

SEQ ID NO: 82 - protein, 8 aa, Artificial sequence
  synthetic sequence
PPANVKYV

SEQ ID NO: 83 - protein, 21 aa, Artificial sequence
  synthetic sequence
FMYVAAAAFVLLFFVGCGVLL

SEQ ID NO: 84 - protein, 21 aa, Artificial sequence
  synthetic sequence
LLYLLAVAVVIILFIILLGVI

SEQ ID NO: 85 - protein, 21 aa, Artificial sequence
  synthetic sequence
LPLLVAGAVLLLVILVLGVMV

SEQ ID NO: 86 - protein, 21 aa, Artificial sequence
  synthetic sequence
PVLCSPVAGVILLALGALLVL

SEQ ID NO: 87 - protein, 21 aa, Artificial sequence
  synthetic sequence
LMYVAAAAFVLLFFVGCGVLL

SEQ ID NO: 88 - protein, 21 aa, Artificial sequence
  synthetic sequence
LLYLLAVAVVIILFFILLGVI

SEQ ID NO: 89 - protein, 21 aa, Artificial sequence
  synthetic sequence
LLPLLVAGAVFLLIIFILGVM

SEQ ID NO: 90 - protein, 21 aa, Artificial sequence
  synthetic sequence
PILCSPVVGVLLLALGALLVL

SEQ ID NO: 91 - protein, 23 aa, Artificial sequence
  synthetic sequence
LHLMYVAAAAFVLLFFVGCGVLL

SEQ ID NO: 92 - protein, 21 aa, Artificial sequence
  synthetic sequence
LLYLLAVAVVIILFLILLGVI

SEQ ID NO: 93 - protein, 21 aa, Artificial sequence
  synthetic sequence
LPLLVAGAVFLLVIFVLGVMV

SEQ ID NO: 94 - protein, 8 aa, Artificial sequence
  synthetic sequence
VGCGVLLS

SEQ ID NO: 95 - protein, 7 aa, Artificial sequence
  synthetic sequence
GCGVLLS

SEQ ID NO: 96 - protein, 333 aa, Artificial sequence
  synthetic sequence
ILDYSFTGGAGRDIPPPQIEEACELPECQVDAGNKVCNLQCNNHACGWDGGDCSLNFNDPWKNCTQSLQCWKYFSDGHCDSQCNSAGCLFDGFDCQLTEGQCNPLYDQYCKDHFSDGHCDQGCNSAECEWDGLDCAEHVPERLAAGTLVLVVLLPPDQLRNNSFHFLRELSHVLHTNVVFKRDAQGQQMIFPYYGHEEELRKHPIKRSTVGWATSSLLPGTSGGRQRRELDPMDIRGSIVYLEIDNRQCVQSSSQCFQSATDVAAFLGALASLGSLNIPYKIEAVKSEPVEPPLPSQLHLMYVAAAAFVLLFFVGCGVLLSRKRRRQLCIQKL

SEQ ID NO: 97 - protein, 370 aa, Artificial sequence
  synthetic sequence
PCVGSNPCYNQGTCEPTSENPFYRCLCPAKFNGLLCHILDYSFTGGAGRDIPPPQIEEACELPECQVDAGNKVCNLQCNNHACGWDGGDCSLNFNDPWKNCTQSLQCWKYFSDGHCDSQCNSAGCLFDGFDCQLTEGQCNPLYDQYCKDHFSDGHCDQGCNSAECEWDGLDCAEHVPERLAAGTLVLVVLLPPDQLRNNSFHFLRELSHVLHTNVVFKRDAQGQQMIFPYYGHEEELRKHPIKRSTVGWATSSLLPGTSGGRQRRELDPMDIRGSIVYLEIDNRQCVQSSSQCFQSATDVAAFLGALASLGSLNIPYKIEAVKSEPVEPPLPSQLHLMYVAAAAFVLLFFVGCGVLLSRKRRRQLCIQKL

SEQ ID NO: 98 - protein, 787 aa, Artificial sequence
  synthetic sequence
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGILDYSFTGGAGRDIPPPQIEEACELPECQVDAGNKVCNLQCNNHACGWDGGDCSLNFNDPWKNCTQSLQCWKYFSDGHCDSQCNSAGCLFDGFDCQLTEGQCNPLYDQYCKDHFSDGHCDQGCNSAECEWDGLDCAEHVPERLAAGTLVLVVLLPPDQLRNNSFHFLRELSHVLHTNVVFKRDAQGQQMIFPYYGHEEELRKHPIKRSTVGWATSSLLPGTSGGRQRRELDPMDIRGSIVYLEIDNRQCVQSSSQCFQSATDVAAFLGALASLGSLNIPYKIEAVKSEPVEPPLPSQLHLMYVAAAAFVLLFFVGCGVLLSRKRRRQLCIQKLMSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGGPADALDDFDLDMLPADALDDFDLDMLPADALDDFDLDMLPG

SEQ ID NO: 99 - protein, 889 aa, Artificial sequence
  synthetic sequence
METDTLLLWVLLLWVPGSTGDYPYDVPDYAGAQPARPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKILDYSFTGGAGRDIPPPQIEEACELPECQVDAGNKVCNLQCNNHACGWDGGDCSLNFNDPWKNCTQSLQCWKYFSDGHCDSQCNSAGCLFDGFDCQLTEGQCNPLYDQYCKDHFSDGHCDQGCNSAECEWDGLDCAEHVPERLAAGTLVLVVLLPPDQLRNNSFHFLRELSHVLHTNVVFKRDAQGQQMIFPYYGHEEELRKHPIKRSTVGWATSSLLPGTSGGRQRRELDPMDIRGSIVYLEIDNRQCVQSSSQCFQSATDVAAFLGALASLGSLNIPYKIEAVKSEPVEPPLPSQLHLMYVAAAAFVLLFFVGCGVLLSRKRRRQLCIQKLMSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGGPADALDDFDLDMLPADALDDFDLDMLPADALDDFDLDMLPG

SEQ ID NO: 100 - protein, 854 aa, Artificial sequence
  synthetic sequence
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSILDYSFTGGAGRDIPPPQIEEACELPECQVDAGNKVCNLQCNNHACGWDGGDCSLNFNDPWKNCTQSLQCWKYFSDGHCDSQCNSAGCLFDGFDCQLTEGQCNPLYDQYCKDHFSDGHCDQGCNSAECEWDGLDCAEHVPERLAAGTLVLVVLLPPDQLRNNSFHFLRELSHVLHTNVVFKRDAQGQQMIFPYYGHEEELRKHPIKRSTVGWATSSLLPGTSGGRQRRELDPMDIRGSIVYLEIDNRQCVQSSSQCFQSATDVAAFLGALASLGSLNIPYKIEAVKSEPVEPPLPSQLHLMYVAAAAFVLLFFVGCGVLLSRKRRRQLCIQKLMSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGGPADALDDFDLDMLPADALDDFDLDMLPADALDDFDLDMLPG

SEQ ID NO: 101 - protein, 854 aa, Artificial sequence; identical to SEQ ID NO: 100
  synthetic sequence

SEQ ID NO: 102 - protein, 854 aa, Artificial sequence
  synthetic sequence
MALPVTALLLPLALLLHAARPDYKDDDDKGSQVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGQGTTVTVSSGGGGSGGGGSSGGGSDIELTQSPAIMSASPGEKVTMTCSASSSVSYMHWYQQKSGTSPKRWIYDTSKLASGVPGRFSGSGSGNSYSLTISSVEAEDDATYYCQQWSKHPLTYGAGTKLEIKASILDYSFTGGAGRDIPPPQIEEACELPECQVDAGNKVCNLQCNNHACGWDGGDCSLNFNDPWKNCTQSLQCWKYFSDGHCDSQCNSAGCLFDGFDCQLTEGQCNPLYDQYCKDHFSDGHCDQGCNSAECEWDGLDCAEHVPERLAAGTLVLVVLLPPDQLRNNSFHFLRELSHVLHTNVVFKRDAQGQQMIFPYYGHEEELRKHPIKRSTVGWATSSLLPGTSGGRQRRELDPMDIRGSIVYLEIDNRQCVQSSSQCFQSATDVAAFLGALASLGSLNIPYKIEAVKSEPVEPPLPSQLHLMYVAAAAFVLLFFVGCGVLLSRKRRRQLCIQKLMSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGGPADALDDFDLDMLPADALDDFDLDMLPADALDDFDLDMLPG

SEQ ID NO: 103 - protein, 871 aa, Artificial sequence
  synthetic sequence
MALPVTALLLPLALLLHAARPDYKDDDDKGSQVQLQQQVQLQESGGDLVKPGGSLKLSCAASGFTFSHYGMSWVRQTPDKRLEWVATIGSRGTYTHYPDSVKGRFTISRDNDKNALYLQMNSLKSEDTAMYYCARRSEFYYYGNTYYYSAMDYWGQGASVTVSSGGGGSGGGGSGGGGSDIVLTQSPAFLAVSLGQRATISCRASESVDNYGFSFMNWFQQKPGQPPKLLIYAISNRGSGVPARFSGSGSGTDFSLNIHPVEEDDPAMYFCQQTKEVPWTFGGGTKLEIKILDYSFTGGAGRDIPPPQIEEACELPECQVDAGNKVCNLQCNNHACGWDGGDCSLNFNDPWKNCTQSLQCWKYFSDGHCDSQCNSAGCLFDGFDCQLTEGQCNPLYDQYCKDHFSDGHCDQGCNSAECEWDGLDCAEHVPERLAAGTLVLVVLLPPDQLRNNSFHFLRELSHVLHTNVVFKRDAQGQQMIFPYYGHEEELRKHPIKRSTVGWATSSLLPGTSGGRQRRELDPMDIRGSIVYLEIDNRQCVQSSSQCFQSATDVAAFLGALASLGSLNIPYKIEAVKSEPVEPPLPSQLHLMYVAAAAFVLLFFVGCGVLLSRKRRRQLCIQKLMSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGGPADALDDFDLDMLPADALDDFDLDMLPADALDDFDLDMLPG

SEQ ID NO: 104 - protein, 873 aa, Artificial sequence
  synthetic sequence
MALPVTALLLPLALLLHAARPEQKLISEEDLGSQVQLQQQVQLQESGGDLVKPGGSLKLSCAASGFTFSHYGMSWVRQTPDKRLEWVATIGSRGTYTHYPDSVKGRFTISRDNDKNALYLQMNSLKSEDTAMYYCARRSEFYYYGNTYYYSAMDYWGQGASVTVSSGGGGSGGGGSGGGGSDIVLTQSPAFLAVSLGQRATISCRASESVDNYGFSFMNWFQQKPGQPPKLLIYAISNRGSGVPARFSGSGSGTDFSLNIHPVEEDDPAMYFCQQTKEVPWTFGGGTKLEIKILDYSFTGGAGRDIPPPQIEEACELPECQVDAGNKVCNLQCNNHACGWDGGDCSLNFNDPWKNCTQSLQCWKYFSDGHCDSQCNSAGCLFDGFDCQLTEGQCNPLYDQYCKDHFSDGHCDQGCNSAECEWDGLDCAEHVPERLAAGTLVLVVLLPPDQLRNNSFHFLRELSHVLHTNVVFKRDAQGQQMIFPYYGHEEELRKHPIKRSTVGWATSSLLPGTSGGRQRRELDPMDIRGSIVYLEIDNRQCVQSSSQCFQSATDVAAFLGALASLGSLNIPYKIEAVKSEPVEPPLPSQLHLMYVAAAAFVLLFFVGCGVLLSRKRRRQLCIQKLMSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGGPADALDDFDLDMLPADALDDFDLDMLPADALDDFDLDMLPG

SEQ ID NO: 105 - protein, 739 aa, Artificial sequence
  synthetic sequence
MALPVTALLLPLALLLHAARPEQKLISEEDLMADVQLVESGGGLVQAGGSLRLSCAASGRTFSTSAMGWFRQAPGKEREFVARITWSAGYTAYSDSVKGRFTISRDKAKNTVYLQMNSLKPEDTAVYYCASRSAGYSSSLTRREDYAYWGQGTQVTVSILDYSFTGGAGRDIPPPQIEEACELPECQVDAGNKVCNLQCNNHACGWDGGDCSLNFNDPWKNCTQSLQCWKYFSDGHCDSQCNSAGCLFDGFDCQLTEGQCNPLYDQYCKDHFSDGHCDQGCNSAECEWDGLDCAEHVPERLAAGTLVLVVLLPPDQLRNNSFHFLRELSHVLHTNVVFKRDAQGQQMIFPYYGHEEELRKHPIKRSTVGWATSSLLPGTSGGRQRRELDPMDIRGSIVYLEIDNRQCVQSSSQCFQSATDVAAFLGALASLGSLNIPYKIEAVKSEPVEPPLPSQLHLMYVAAAAFVLLFFVGCGVLLSRKRRRQLCIQKLMSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGGPADALDDFDLDMLPADALDDFDLDMLPADALDDFDLDMLPG

SEQ ID NO: 106 - protein, 740 aa, Artificial sequence
  synthetic sequence
MALPVTALLLPLALLLHAARPEQKLISEEDLMADVQLVESGGGLVQAGGSLRLSCAASGRTISMAAMSWFRQAPGKEREFVAGISRSAGSAVHADSVKGRFTISRDNTKNTLYLQMNSLKAEDTAVYYCAVRTSGFFGSIPRTGTAFDYWGQGTQVTVSILDYSFTGGAGRDIPPPQIEEACELPECQVDAGNKVCNLQCNNHACGWDGGDCSLNFNDPWKNCTQSLQCWKYFSDGHCDSQCNSAGCLFDGFDCQLTEGQCNPLYDQYCKDHFSDGHCDQGCNSAECEWDGLDCAEHVPERLAAGTLVLVVLLPPDQLRNNSFHFLRELSHVLHTNVVFKRDAQGQQMIFPYYGHEEELRKHPIKRSTVGWATSSLLPGTSGGRQRRELDPMDIRGSIVYLEIDNRQCVQSSSQCFQSATDVAAFLGALASLGSLNIPYKIEAVKSEPVEPPLPSQLHLMYVAAAAFVLLFFVGCGVLLSRKRRRQLCIQKLMSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGGPADALDDFDLDMLPADALDDFDLDMLPADALDDFDLDMLPG

SEQ ID NO: 107 - protein, 740 aa, Artificial sequence
  synthetic sequence
MALPVTALLLPLALLLHAARPEQKLISEEDLMAQVQLVESGGGLVQTGGSLKLSCTASVRTLSYYHVGWFRQAPGKEREFVAGIHRSGESTFYADSVKGRFTISRDNAKNTVHLQMNSLKPEDTAVYYCAQRVRGFFGPLRSTPSWYDYWGQGTQVTVSILDYSFTGGAGRDIPPPQIEEACELPECQVDAGNKVCNLQCNNHACGWDGGDCSLNFNDPWKNCTQSLQCWKYFSDGHCDSQCNSAGCLFDGFDCQLTEGQCNPLYDQYCKDHFSDGHCDQGCNSAECEWDGLDCAEHVPERLAAGTLVLVVLLPPDQLRNNSFHFLRELSHVLHTNVVFKRDAQGQQMIFPYYGHEEELRKHPIKRSTVGWATSSLLPGTSGGRQRRELDPMDIRGSIVYLEIDNRQCVQSSSQCFQSATDVAAFLGALASLGSLNIPYKIEAVKSEPVEPPLPSQLHLMYVAAAAFVLLFFVGCGVLLSRKRRRQLCIQKLMSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGGPADALDDFDLDMLPADALDDFDLDMLPADALDDFDLDMLPG

SEQ ID NO: 108 - protein, 882 aa, Artificial sequence
  synthetic sequence
MALPVTALLLPLALLLHAARPEQKLISEEDLMAQVQLVESGGRLVQAGDSLRLSCAASGRTFSTSAMAWFRQAPGREREFVAAITWTVGNTILGDSVKGRFTISRDRAKNTVDLQMDNLEPEDTAVYYCSARSRGYVLSVLRSVDSYDYWGQGTQVTVSGGGGSGGGGSGGGGSMAQVQLVESGGGLVQAGGSLRLSCAASGRTFSNYAMGWFRQAPGKEREFVAAISWTGVSTYYADSVKGRFTISRDNDKNTVYVQMNSLIPEDTAIYYCAAVRARSFSDTYSRVNEYDYWGQGTQVTVILDYSFTGGAGRDIPPPQIEEACELPECQVDAGNKVCNLQCNNHACGWDGGDCSLNFNDPWKNCTQSLQCWKYFSDGHCDSQCNSAGCLFDGFDCQLTEGQCNPLYDQYCKDHFSDGHCDQGCNSAECEWDGLDCAEHVPERLAAGTLVLVVLLPPDQLRNNSFHFLRELSHVLHTNVVFKRDAQGQQMIFPYYGHEEELRKHPIKRSTVGWATSSLLPGTSGGRQRRELDPMDIRGSIVYLEIDNRQCVQSSSQCFQSATDVAAFLGALASLGSLNIPYKIEAVKSEPVEPPLPSQLHLMYVAAAAFVLLFFVGCGVLLSRKRRRQLCIQKLMSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGGPADALDDFDLDMLPADALDDFDLDMLPADALDDFDLDMLPG

SEQ ID NO: 109 - protein, 817 aa, Artificial sequence
  synthetic sequence
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSILDYSFTGGAGRDIPPPQIEEACELPECQVDAGNKVCNLQCNNHACGWDGGDCSLNFNDPWKNCTQSLQCWKYFSDGHCDSQCNSAGCLFDGFDCQLTEGQCNPLYDQYCKDHFSDGHCDQGCNSAECEWDGLDCAEHVPERLAAGTLVLVVLLPPDQLRNNSFHFLRELSHVLHTNVVFKRDAQGQQMIFPYYGHEEELRKHPIKRSTVGWATSSLLPGTSGGRQRRELDPMDIRGSIVYLEIDNRQCVQSSSQCFQSATDVAAFLGALASLGSLNIPYKIEAVKSEPVEPPLPSQLHLMYVAAAAFVLLFFVGCGVLLSRKRRRQLCIQKLMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 110 - protein, 809 aa, Artificial sequence
  synthetic sequence
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSILDYSFTGGAGRDIPPPQIEEACELPECQVDAGNKVCNLQCNNHACGWDGGDCSLNFNDPWKNCTQSLQCWKYFSDGHCDSQCNSAGCLFDGFDCQLTEGQCNPLYDQYCKDHFSDGHCDQGCNSAECEWDGLDCAEHVPERLAAGTLVLVVLLPPDQLRNNSFHFLRELSHVLHTNVVFKRDAQGQQMIFPYYGHEEELRKHPIKRSTVGWATSSLLPGTSGGRQRRELDPMDIRGSIVYLEIDNRQCVQSSSQCFQSATDVAAFLGALASLGSLNIPYKIEAVKSEPVEPPLPSQLHLMYVAAAAFVLLFFVGCGVLLSRKRRRQLCILLEIRAAFLRQRNTALRTEVAELEQEVQRLENEVSQYETRYGPLGGGKGGSGGSGGSMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAA

SEQ ID NO: 111 - protein, 719 aa, Artificial sequence
  synthetic sequence
MALPVTALLLPLALLLHAARPDYKDDDDKGSQVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGQGTTVTVSSGGGGSGGGGSSGGGSDIELTQSPAIMSASPGEKVTMTCSASSSVSYMHWYQQKSGTSPKRWIYDTSKLASGVPGRFSGSGSGNSYSLTISSVEAEDDATYYCQQWSKHPLTYGAGTKLEIKASILDYSFTGGAGRDIPPPQIEEACELPECQVDAGNKVCNLQCNNHACGWDGGDCSLNFNDPWKNCTQSLQCWKYFSDGHCDSQCNSAGCLFDGFDCQLTEGQCNPLYDQYCKDHFSDGHCDQGCNSAECEWDGLDCAEHVPERLAAGTLVLVVLLPPDQLRNNSFHFLRELSHVLHTNVVFKRDAQGQQMIFPYYGHEEELRKHPIKRSTVGWATSSLLPGTSGGRQRRELDPMDIRGSIVYLEIDNRQCVQSSSQCFQSATDVAAFLGALASLGSLNIPYKIEAVKSEPVEPPLPSQLHLMYVAAAAFVLLFFVGCGVLLSRKRRRQLCILPKKKRKVDALDDFDLDMLGSDALDDFDLDMLGSDALDDFDLDMLGSDALDDFDLDMLGSGGSGGSGGSLEIEAAFLERENTALETRVAELRQRVQRLRNRVSQYRTRYGPLGGGK

SEQ ID NO: 112 - protein, 272 aa, Homo sapiens
MDSYLLMWGLLTFIMVPGCQAELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQVAVAGCVFLLISVLLLSGLTWQRRQRKSRRTI

SEQ ID NO: 113 - protein, 551 aa, Homo sapiens
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 114 - protein, 369 aa, Homo sapiens
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

(19) United States
(12) Patent Application Publication
(10) Pub. No.: US 2022/0265854 A1
(43) Pub. Date: Aug. 25, 2022

(54) SYNTHETIC IMMUNE CELLS AND METHODS OF USE THEREOF

(71) Applicant: The Regents of the University of California, Oakland, CA (US)

(72) Inventors: Wendell A. Lim, San Francisco, CA (US); Nicholas W. Frankel, San Francisco, CA (US)

(21) Appl. No.: 17/630,875

(22) PCT Filed: Sep. 15, 2020
(86) PCT No.: PCT/US2020/050838

§ 371 Date: Jan. 27, 2022

Related U.S. Application Data
(60) Provisional application No. 62/901,999, filed on Sep. 18, 2019.

(51) Int. Cl. A61K 48/00; A61K 35/15; A61K 35/17; C07K 14/54; C07K 14/55; C07K 14/705

(52) U.S. Cl. CPC A61K 48/005; A61K 35/15; A61K 35/17; C07K 14/5418; C07K 14/5443; C07K 14/55; C07K 14/705; C07K 2317/622; C07K 2319/03; C07K 2319/33

(57) ABSTRACT

The present disclosure provides a genetically modified, in vitro immune cell. The immune cell is genetically modified with one or more nucleic acids comprising nucleotide sequences encoding: a) a chimeric polypeptide comprising: i) an antibody specific for a target antigen; and ii) a binding triggered transcriptional activator; and b) a cytokine or proliferation-inducing polypeptide that increases proliferation and/or activity of an effector immune cell, where the nucleotide sequence encoding the cytokine or proliferation-inducing polypeptide is operably linked to a transcriptional control element responsive to the transcriptional activator. The present disclosure provides compositions comprising the genetically modified, in vitro immune cell; and treatment methods comprising administration of the genetically modified, in vitro immune cell.

### CROSS-REFERENCING

This application claims the benefit of U.S. provisional application Ser. No. 62/901,999, filed on Sep. 18, 2019, which application is incorporated by reference herein.

### STATEMENT REGARDING FEDERALLY SPONSORED RESEARCH

This invention was made with government support under grant no. R01 CA196277 awarded by The National Institutes of Health. The government has certain rights in the invention.

### INTRODUCTION

Immune cell proliferation plays a central role in generating potent immune responses, including those of chimeric antigen receptor (CAR)-T cells. Extending cell therapy beyond treatment of blood cancers to solid cancers will require tools to enhance immune cell proliferation locally without systemic side effects.

There is a need in the art for synthetic immune cells that inducibly secrete proliferative cytokines upon recognition of a local target antigen, such as a tumor antigen.

### SUMMARY

The present disclosure provides a genetically modified, in vitro immune cell. The immune cell is genetically modified with one or more nucleic acids comprising nucleotide sequences encoding: a) a chimeric polypeptide comprising: i) an antibody specific for a target antigen; and ii) a binding triggered transcriptional activator; and b) a cytokine or proliferation-inducing polypeptide that increases proliferation and/or activity of an effector immune cell, where the nucleotide sequence encoding the cytokine or proliferation-inducing polypeptide is operably linked to a transcriptional control element responsive to the transcriptional activator. The present disclosure provides compositions comprising the genetically modified, in vitro immune cell; and treatment methods comprising administration of the genetically modified, in vitro immune cell.

### BRIEF DESCRIPTION OF THE DRAWINGS

FIG. 1A-1C depict schematically immune cell expansion and balance between amplification and side effects.

FIG. 2A-2D depict synthetic helper T cells with inducible cytokine circuits for immune cell expansion.

FIG. 3A-3E depict the effect of synthetic helper T cells on locally targeted proliferation in vivo.

FIG. 4A-4C depict bioluminescence images of individual mice over 18 days.

FIG. 5A-5D depict synthetic helpers forming a functional AND-gate circuit with cytotoxic effectors to specifically enhance local killing of target tumors.

FIG. 6A-6B depict tumor volume trajectories of individual mice.

FIG. 7 is a schematic depiction of a chimeric Notch receptor polypeptide.

FIG. 8A-8G provide schematic depictions of exemplary chimeric Notch receptor polypeptides.

FIG. 9A-9C depict examples of chimeric Notch receptor polypeptides. The sequences are set forth in SEQ ID NOs: 96-98.

FIG. 10A-10C provide amino acid sequences of exemplary chimeric Notch receptor polypeptides. The sequences are set forth in SEQ ID NOs: 99-101.

FIG. 11 provides an amino acid sequence of an exemplary chimeric Notch polypeptide. The sequence is set forth in SEQ ID NO: 102.

FIG. 12A-12B provide amino acid sequences of exemplary chimeric Notch receptor polypeptides. The sequences is set forth in SEQ ID NOs: 103-104.

FIG. 13A-13D provide amino acid sequences of exemplary chimeric Notch receptor polypeptides. The sequences are set forth in SEQ ID NOs: 105-108.

FIGS. 14-16 provide amino acid sequences of exemplary chimeric Notch receptor polypeptides. The sequences are set forth in SEQ ID NOs: 109-111.

FIG. 17A-17C depicts amino acid sequence of IL-2 receptor polypeptides IL-2Rα, IL2Rβ, and IL-2Rγ. The sequences are set forth in SEQ ID NOs: 112-114.

### DEFINITIONS

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

“Operably linked” refers to a juxtaposition wherein the components so described are in a relationship permitting them to function in their intended manner For instance, a promoter is operably linked to a coding sequence if the promoter affects its transcription or expression. Operably linked nucleic acid sequences may but need not necessarily be adjacent. For example, in some instances a coding sequence operably linked to a promoter may be adjacent to the promoter. In some instances, a coding sequence operably linked to a promoter may be separated by one or more intervening sequences, including coding and non-coding sequences. Also, in some instances, more than two sequences may be operably linked including but not limited to e.g., where two or more coding sequences are operably linked to a single promoter.

A “vector” or “expression vector” is a replicon, such as plasmid, phage, virus, or cosmid, to which another DNA segment, i.e. an “insert”, may be attached so as to bring about the replication of the attached segment in a cell.

“Heterologous,” as used herein, means a nucleotide or polypeptide sequence that is not found in the native (e.g., naturally-occurring) nucleic acid or protein, respectively. Heterologous nucleic acids or polypeptide may be derived from a different species as the organism or cell within which the nucleic acid or polypeptide is present or is expressed. Accordingly, a heterologous nucleic acids or polypeptide is generally of unlike evolutionary origin as compared to the cell or organism in which it resides.

The terms “antibodies” and “immunoglobulin” include antibodies or immunoglobulins of any isotype, fragments of antibodies that retain specific binding to antigen, including, but not limited to, Fab, Fv, scFv, and Fd fragments, chimeric antibodies, humanized antibodies, single-chain antibodies (scAb), single domain antibodies (dAb), single domain heavy chain antibodies, a single domain light chain antibodies, nanobodies, bi-specific antibodies, multi-specific antibodies, and fusion proteins comprising an antigen-binding (also referred to herein as antigen binding) portion of an antibody and a non-antibody protein. The antibodies can be detectably labeled, e.g., with a radioisotope, an enzyme that generates a detectable product, a fluorescent protein, and the like. The antibodies can be further conjugated to other moieties, such as members of specific binding pairs, e.g., biotin (member of biotin-avidin specific binding pair), and the like. The antibodies can also be bound to a solid support, including, but not limited to, polystyrene plates or beads, and the like. Also encompassed by the term are Fab′, Fv, F(ab′)₂, and or other antibody fragments that retain specific binding to antigen, and monoclonal antibodies. As used herein, a monoclonal antibody is an antibody produced by a group of identical cells, all of which were produced from a single cell by repetitive cellular replication. That is, the clone of cells only produces a single antibody species. While a monoclonal antibody can be produced using hybridoma production technology, other production methods known to those skilled in the art can also be used (e.g., antibodies derived from antibody phage display libraries). An antibody can be monovalent or bivalent. An antibody can be an Ig monomer, which is a “Y-shaped” molecule that consists of four polypeptide chains: two heavy chains and two light chains connected by disulfide bonds.

The term “humanized immunoglobulin” as used herein refers to an immunoglobulin comprising portions of immunoglobulins of different origin, wherein at least one portion comprises amino acid sequences of human origin. For example, the humanized antibody can comprise portions derived from an immunoglobulin of nonhuman origin with the requisite specificity, such as a mouse, and from immunoglobulin sequences of human origin (e.g., chimeric immunoglobulin), joined together chemically by conventional techniques (e.g., synthetic) or prepared as a contiguous polypeptide using genetic engineering techniques (e.g., DNA encoding the protein portions of the chimeric antibody can be expressed to produce a contiguous polypeptide chain). Another example of a humanized immunoglobulin is an immunoglobulin containing one or more immunoglobulin chains comprising a complementarity-determining region (CDR) derived from an antibody of nonhuman origin and a framework region derived from a light and/or heavy chain of human origin (e.g., CDR-grafted antibodies with or without framework changes). Chimeric or CDR-grafted single chain antibodies are also encompassed by the term humanized immunoglobulin. See, e.g., Cabilly et al., U.S. Pat. No. 4,816,567; Cabilly et al., European Patent No. 0,125,023 B1; Boss et al., U.S. Pat. No. 4,816,397; Boss et al., European Patent No. 0,120,694 B1; Neuberger, M. S. et al., WO 86/01533; Neuberger, M. S. et al., European Patent No. 0,194,276 B1; Winter, U.S. Pat. No. 5,225,539; Winter, European Patent No. 0,239,400 B1; Padlan, E. A. et al., European Patent Application No. 0,519,596 A1. See also, Ladner et al., U.S. Pat. No. 4,946,778; Huston, U.S. Pat. No. 5,476,786; and Bird, R. E. et al., Science, 242: 423-426 (1988)), regarding single chain antibodies.

The term “nanobody” (Nb), as used herein, refers to the smallest antigen binding fragment or single variable domain (V_{HH}) derived from naturally occurring heavy chain antibody and is known to the person skilled in the art. They are derived from heavy chain only antibodies, seen in camelids (Hamers-Casterman et al., 1993; Desmyter et al., 1996). In the family of “camelids” immunoglobulins devoid of light polypeptide chains are found. “Camelids” comprise old world camelids (Camelus bactrianus and Camelus dromedarius) and new world camelids (for example, Llama paccos, Llama glama, Llama guanicoe and Llama vicugna). A single variable domain heavy chain antibody is referred to herein as a nanobody or a V_{HH} antibody.

“Antibody fragments” comprise a portion of an intact antibody, for example, the antigen binding or variable region of the intact antibody. Examples of antibody fragments include Fab, Fab′, F(ab′)₂, and Fv fragments; diabodies; linear antibodies (Zapata et al., Protein Eng. 8(10): 1057-1062 (1995)); domain antibodies (dAb; Holt et al. (2003) Trends Biotechnol. 21:484); single-chain antibody molecules; and multi-specific antibodies formed from antibody fragments. Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′)₂ fragment that has two antigen combining sites and is still capable of cross-linking antigen.

“Fv” is the minimum antibody fragment that contains a complete antigen-recognition and—binding site. This region consists of a dimer of one heavy—and one light-chain variable domain in tight, non-covalent association. It is in this configuration that the three CDRS of each variable domain interact to define an antigen-binding site on the surface of the V_{H}-V_{L} dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The “Fab” fragment also contains the constant domain of the light chain and the first constant domain (CH,) of the heavy chain. Fab fragments differ from Fab' fragments by the addition of a few residues at the carboxyl terminus of the heavy chain CH₁ domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The “light chains” of antibodies (immunoglobulins) from any vertebrate species can be assigned to one of two clearly distinct types, called kappa and lambda, based on the amino acid sequences of their constant domains. Depending on the amino acid sequence of the constant domain of their heavy chains, immunoglobulins can be assigned to different classes. There are five major classes of immunoglobulins: IgA, IgD, IgE, IgG, and IgM, and several of these classes can be further divided into subclasses (isotypes), e.g., IgG1, IgG2, IgG3, IgG4, IgA, and IgA2. The subclasses can be further divided into types, e.g., IgG2a and IgG2b.

“Single-chain Fv” or “sFv” or “scFv” antibody fragments comprise the V_{H} and V_{L} domains of antibody, wherein these domains are present in a single polypeptide chain. In some embodiments, the Fv polypeptide further comprises a polypeptide linker between the V_{H} and V_{L} domains, which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a heavy-chain variable domain (V_{H}) connected to a light-chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al. (1993) Proc. Natl. Acad. Sci. USA 90:6444-6448.

As used herein, the term “affinity” refers to the equilibrium constant for the reversible binding of two agents (e.g., an antibody and an antigen) and is expressed as a dissociation constant (K_{D}). Affinity can be at least 1-fold greater, at least 2-fold greater, at least 3-fold greater, at least 4-fold greater, at least 5-fold greater, at least 6-fold greater, at least 7-fold greater, at least 8-fold greater, at least 9-fold greater, at least 10-fold greater, at least 20-fold greater, at least 30-fold greater, at least 40-fold greater, at least 50-fold greater, at least 60-fold greater, at least 70-fold greater, at least 80-fold greater, at least 90-fold greater, at least 100-fold greater, or at least 1,000-fold greater, or more, than the affinity of an antibody for unrelated amino acid sequences. Affinity of an antibody to a target protein can be, for example, from about 100 nanomolar (nM) to about 0.1 nM, from about 100 nM to about 1 picomolar (pM), or from about 100 nM to about 1 femtomolar (fM) or more. As used herein, the term “avidity” refers to the resistance of a complex of two or more agents to dissociation after dilution. The terms “immunoreactive” and “preferentially binds” are used interchangeably herein with respect to antibodies and/or antigen-binding fragments.

The term “binding” refers to a direct association between two molecules, due to, for example, covalent, electrostatic, hydrophobic, and ionic and/or hydrogen-bond interactions, including interactions such as salt bridges and water bridges. In some cases, a specific binding member present in the extracellular domain of a chimeric polypeptide of the present disclosure binds specifically to its binding partner, such as an antigen or a peptide-major histocompatibility complex (peptide-MHC). “Specific binding” refers to binding with an affinity of at least about 10⁻⁷ M or greater, e.g., 5×10⁻⁷ M, 10⁻⁸ M, 5×10⁻⁸ M, and greater. “Non-specific binding” refers to binding with an affinity of less than about 10⁻⁷ M, e.g., binding with an affinity of 10⁻⁶ M, 10⁻⁵ M, 10⁻⁴ M, etc.

The terms “polypeptide,” “peptide,” and “protein”, used interchangeably herein, refer to a polymeric form of amino acids of any length, which can include genetically coded and non-genetically coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; and the like.

An “isolated” polypeptide is one that has been identified and separated and/or recovered from a component of its natural environment. Contaminant components of its natural environment are materials that would interfere with diagnostic or therapeutic uses for the polypeptide, and may include enzymes, hormones, and other proteinaceous or nonproteinaceous solutes. In some cases, the polypeptide will be purified (1) to greater than 90%, greater than 95%, or greater than 98%, by weight of antibody as determined by the Lowry method, for example, more than 99% by weight, (2) to a degree sufficient to obtain at least 15 residues of N-terminal or internal amino acid sequence by use of a spinning cup sequenator, or (3) to homogeneity by sodium dodecyl sulfate-polyacrylamide gel electrophoresis (SDS-PAGE) under reducing or nonreducing conditions using Coomassie blue or silver stain. Isolated polypeptide includes the polypeptide in situ within recombinant cells since at least one component of the polypeptide's natural environment will not be present. In some instances, isolated polypeptide will be prepared by at least one purification step.

The terms “chimeric antigen receptor” and “CAR”, used interchangeably herein, refer to artificial multi-module molecules capable of triggering or inhibiting the activation of an immune cell which generally but not exclusively comprise an extracellular domain (e.g., a ligand/antigen binding domain), a transmembrane domain and one or more intracellular signaling domains. The term CAR is not limited specifically to CAR molecules but also includes CAR variants. CAR variants include split CARs wherein the extracellular portion (e.g., the ligand binding portion) and the intracellular portion (e.g., the intracellular signaling portion) of a CAR are present on two separate molecules. CAR variants also include ON-switch CARs which are conditionally activatable CARs, e.g., comprising a split CAR wherein conditional hetero-dimerization of the two portions of the split CAR is pharmacologically controlled (e.g., as described in PCT publication no. WO 2014/127261 and US Patent Application No. 2015/0368342, the disclosures of which are incorporated herein by reference in their entirety). CAR variants also include bispecific CARs, which include a secondary CAR binding domain that can either amplify or inhibit the activity of a primary CAR. CAR variants also include inhibitory chimeric antigen receptors (iCARs) which may, e.g., be used as a component of a bispecific CAR system, where binding of a secondary CAR binding domain results in inhibition of primary CAR activation. CAR molecules and derivatives thereof (i.e., CAR variants) are described, e.g., in PCT Application No. US2014/016527; Fedorov et al. Sci Transl Med (2013); 5(215):215ra172; Glienke et al. Front Pharmacol (2015) 6:21; Kakarla & Gottschalk 52 Cancer J (2014) 20(2):151-5; Riddell et al. Cancer J (2014) 20(2):141-4; Pegram et al. Cancer J (2014) 20(2):127-33; Cheadle et al. Immunol Rev (2014) 257(1):91-106; Barrett et al. Annu Rev Med (2014) 65:333-47; Sadelain et al. Cancer Discov (2013) 3(4):388-98; Cartellieri et al., J Biomed Biotechnol (2010) 956304; the disclosures of which are incorporated herein by reference in their entirety. Useful CARs also include the anti-CD19-4-1BB-CD3ζ CAR expressed by lentivirus loaded CTL019 (Tisagenlecleucel-T) CAR-T cells as commercialized by Novartis (Basel, Switzerland).

As used herein, the terms “treatment,” “treating,” “treat” and the like, refer to obtaining a desired pharmacologic and/or physiologic effect. The effect can be prophylactic in terms of completely or partially preventing a disease or symptom thereof and/or can be therapeutic in terms of a partial or complete cure for a disease and/or adverse effect attributable to the disease. “Treatment,” as used herein, covers any treatment of a disease in a mammal, particularly in a human, and includes: (a) preventing the disease from occurring in a subject which can be predisposed to the disease but has not yet been diagnosed as having it; (b) inhibiting the disease, i.e., arresting its development; and (c) relieving the disease, i.e., causing regression of the disease.

A “therapeutically effective amount” or “efficacious amount” refers to the amount of an agent, or combined amounts of two agents, that, when administered to a mammal or other subject for treating a disease, is sufficient to effect such treatment for the disease. The “therapeutically effective amount” will vary depending on the agent(s), the disease and its severity and the age, weight, etc., of the subject to be treated.

The terms “individual,” “subject,” “host,” and “patient,” used interchangeably herein, refer to a mammal, including, but not limited to, murines (e.g., rats, mice), non-human primates, humans, canines, felines, ungulates (e.g., equines, bovines, ovines, porcines, caprines), lagomorphs, etc. In some cases, the individual is a human In some cases, the individual is a non-human primate. In some cases, the individual is a rodent, e.g., a rat or a mouse. In some cases, the individual is a lagomorph, e.g., a rabbit.

As used herein, the term “immune cells” generally includes white blood cells (leukocytes) which are derived from hematopoietic stem cells (HSC) produced in the bone marrow “Immune cells” includes, e.g., lymphocytes (T cells, B cells, natural killer (NK) cells) and myeloid-derived cells (neutrophil, eosinophil, basophil, monocyte, macrophage, dendritic cells).

“T cell” includes all types of immune cells expressing CD3 including T-helper cells (CD4⁺ cells), cytotoxic T-cells (CD8⁺ cells), T-regulatory cells (Treg) and gamma-delta T cells.

A “cytotoxic cell” includes CD8⁺ T cells, natural-killer (NK) cells, and neutrophils, which cells are capable of mediating cytotoxicity responses.

The term “synthetic,” as used herein in the context of a “synthetic immune cell,” generally refers to an artificially derived (e.g., laboratory generated) cell that is not naturally occurring.

The term “recombinant”, as used herein describes a nucleic acid molecule, e.g., a polynucleotide of genomic, cDNA, viral, semisynthetic, and/or synthetic origin, which, by virtue of its origin or manipulation, is not associated with all or a portion of the polynucleotide sequences with which it is associated in nature. The term recombinant as used with respect to a protein or polypeptide means a polypeptide produced by expression from a recombinant polynucleotide. The term recombinant as used with respect to a host cell or a virus means a host cell or virus into which a recombinant polynucleotide has been introduced. Recombinant is also used herein to refer to, with reference to material (e.g., a cell, a nucleic acid, a protein, or a vector) that the material has been modified by the introduction of a heterologous material (e.g., a cell, a nucleic acid, a protein, or a vector).

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a synthetic immune cell” includes a plurality of such cells and reference to “the IL-2 polypeptide” includes reference to one or more IL-2 polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides a genetically modified, in vitro immune cell. The immune cell is genetically modified with one or more nucleic acids comprising nucleotide sequences encoding: a) a chimeric polypeptide comprising: i) an antibody specific for a target antigen; and ii) a binding triggered transcriptional activator; and b) a cytokine or proliferation-inducing polypeptide that increases proliferation and/or activity of an effector immune cell, where the nucleotide sequence encoding the cytokine or proliferation-inducing polypeptide is operably linked to a transcriptional control element responsive to the transcriptional activator. The present disclosure provides compositions comprising the genetically modified, in vitro immune cell; and treatment methods comprising administration of the genetically modified, in vitro immune cell.

### Genetically Modified Immune Cells

The present disclosure provides a genetically modified, in vitro immune cell. The immune cell is genetically modified with one or more nucleic acids comprising nucleotide sequences encoding: a) a chimeric polypeptide comprising: i) an antibody specific for a target antigen; and ii) a binding triggered transcriptional activator; and b) a cytokine or proliferation-inducing polypeptide that increases proliferation and/or activity of an effector immune cell, where the nucleotide sequence encoding the cytokine or proliferation-inducing polypeptide is operably linked to a transcriptional control element responsive to the transcriptional activator. A genetically modified, in vitro immune cell of the present disclosure is also referred to herein as a “synthetic immune cell.”

A genetically modified, in vitro immune cell of the present disclosure can be a T cell or a macrophage. A genetically modified, in vitro immune cell of the present disclosure can be a cytotoxic T cell. A genetically modified, in vitro immune cell of the present disclosure can be a CD8⁺ T cell. A genetically modified, in vitro immune cell of the present disclosure can be a CD4⁺ T cell.

A genetically modified, in vitro immune cell of the present disclosure, when administered to an individual in need thereof, provides for an increase in proliferation and/or activity of a second immune cell (a “target immune cell”) present in the individual. The second immune cell can be an endogenous immune cell, e.g., an immune cell that is not genetically modified (e.g., not genetically modified ex vivo). For example, the second immune cell can be a T cell that expresses an endogenous T-cell receptor (TCR). The second immune cell can be one that has been genetically modified in vitro and administered to the individual. For example, the second immune cell can be one that has been genetically modified in vitro to express: i) an exogenous TCR; ii) a chimeric antigen receptor (CAR); or iii) a bispecific T-cell engager (BiTE).

In some cases, a genetically modified, in vitro immune cell of the present disclosure, when administered to an individual in need thereof, provides for an increase in proliferation of a target immune cell in the individual of at least 10%, at least 15%, at least 20%, at least 25%, at least 30%, at least 35%, at least 40%, at least 45%, at least 50%, at least 75%, at least 100% (or 2-fold), at least 2.5-fold, at least 5-fold, at least 10-fold, at least 25-fold, at least 50-fold, at least 100-fold, or more than 100-fold, compared to the level of proliferation of the target cell before administration of the genetically modified immune cell of the present disclosure.

In some cases, a genetically modified, in vitro immune cell of the present disclosure, when administered to an individual in need thereof, provides for an increase in cytotoxic T cell activity of a target immune cell in the individual of at least 10%, at least 15%, at least 20%, at least 25%, at least 30%, at least 35%, at least 40%, at least 45%, at least 50%, at least 75%, at least 100% (or 2-fold), at least 2.5-fold, at least 5-fold, at least 10-fold, at least 25-fold, at least 50-fold, at least 100-fold, or more than 100-fold, compared to the cytotoxic activity of the target immune cell before administration of the genetically modified immune cell of the present disclosure.

### Cytokines

As noted above, a genetically modified, in vitro immune cell of the present disclosure is genetically modified with a nucleic acid comprising a nucleotide sequence encoding a cytokine or other proliferation-inducing polypeptide, where the nucleotide sequence encoding the cytokine or proliferation-inducing polypeptide is operably linked to a transcriptional control element responsive to the transcriptional activator present in the chimeric polypeptide.

As used herein, the term “cytokine” includes cytokines comprising naturally-occurring (“wild-type”) amino acid sequences and cytokines comprising non-naturally-occurring amino acid sequences. For example, a cytokine comprising a non-naturally-occurring amino acid sequence can be referred to as a “variant” cytokine. In some cases, a variant cytokine comprises an amino acid sequence that differs from a naturally-occurring amino acid sequence by from 1 to 25 amino acids, e.g., from 1 amino acid to 5 amino acids, from 5 amino acids to 10 amino acids, from 10 amino acids to 15 amino acids, from 15 amino acids to 20 amino acids, or from 20 amino acids to 25 amino acids.

### Variant IL-2 Polypeptides

In some cases, a variant IL-2 polypeptide is one that exhibits increased affinity for IL-2 receptor (IL-2R)-beta (IL-2Rβ). See, e.g., Levin et al. (2012) Nature 484:529; and US 2019/0248860.

An amino acid sequence of wild-type human IL-2, with the signal peptide (in bold and underlined), is as follows:

(SEQ ID NO: 1)
MYRMQLLSCIALSLALVTNSAPTSSSTKKT QLQLEHLLLD LQMILNGINN
YKNPKLTRML TFKFYMPKKA TELKHLQCLE EELKPLEEVL NLAQSKNFHL RPRDLISNIN
VIVLELKGSE TTFMCEYADE TATIVEFLNR WITFCQSIIS TLT.

In some cases, the IL-2 lacks the signal sequence. For example, in some cases, the IL-2 comprises the following amino acid sequence:

APTSSSTKKT QLQLEHLLLD LQMILNGINN YKNPKLTRML TFKFYMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:2).

In some cases, the IL-2 is a variant IL-2 that comprises L80F, R81D, L85V, I86V, and I92F substitutions, based on the amino acid numbering of SEQ ID NO:2. Thus, e.g., in some cases, the variant IL-2 comprises the following amino acid sequence:

(SEQ ID NO: 4)
MYRMQLLSCIALSLALVTNSAPTSSSTKKT QLQLEHLLLD LQMILNGINN
YKNPKLTRML TFKFYMPKKA TELKHLQCLE EELKPLEEVL NLAQSKNFHF DPRDVVISNI
NVFVLELKGSE TTFMCEYADE TATIVEFLNR WITFCQSIIS TLT.

In some cases, the IL-2 is a variant IL-2 that comprises L80F, R81D, L85V, I86V, and I92F substitutions, based on the amino acid numbering of SEQ ID NO:2; and lacks a signal sequence. For example, in some cases, the variant IL-2 comprises the following amino acid sequence:

APTSSSTKKT QLQLEHLLLD LQMILNGINN YKNPKLTRML TFKFYMPKKA TELKHLQCLE EELKPLEEVL NLAQSKNFHF DPRDVVISNIN VFVLELKGSE TTFMCEYADE TATIVEFLNR WITFCQSIIS TLT (SEQ ID NO:5); and has a length of 133 amino acids. Such a variant IL-2 is also referred to as “super 2.” Such a variant IL-2 exhibits higher affinity for IL-2Rβ than an IL-2 polypeptide comprising the amino acid sequence set forth in SEQ ID NO:2. For example, such a variant IL-2 exhibits an affinity for IL-2Rβ that is at least 20%, at least 25%, at least 50%, at least 100% (or 2-fold), at least 2.5-fold, at least 5-fold, or at least 10-fold, higher than the affinity of the IL-2 polypeptide of SEQ ID NO:2 for IL-2Rβ.

In some cases, the variant IL-2 includes, in addition to L80F, R81D, L85V, I86V, and I92F substitutions, an amino acid substitution at one or more of I24, F42, K43, P65, Q74, I89, and V93, based on the amino acid numbering of SEQ ID NO:2. In some cases, the variant IL-2 further includes a K43N amino acid substitution, based on the amino acid numbering of SEQ ID NO:2. In some cases, the variant IL-2 includes, in addition to L80F, R81D, L85V, I86V, and I92F substitutions, one or more amino acid substitutions selected from I24V, P65H, Q74R, Q74H, Q74N, Q74S, I89V, and V93I, based on the amino acid numbering of SEQ ID NO:2.

In some cases, the variant IL-2 comprises the following amino acid substitutions: F42A, L80F, R81D, L85V, I86V, I89V, and I92F, based on the amino acid numbering of SEQ ID NO:2. Thus, for example, in some cases, the variant IL-2 comprises the following amino acid sequence: APTSSSTKKT QLQLEHLLLD LQMILNGINN YKNPKLTRML TAKFYMPKKA TELKHLQCLE EELKPLEEVL NLAQSKNFHF DPRDVVISNVN VFVLELKGSE TTFMCEYADE TATIVEFLNR WITFCQSIIS TLT (SEQ ID NO:6).

In some cases, the variant IL-2 comprises the following amino acid substitutions: L80F, R81D, L85V, I86V, I89V, 192F, and V93I, based on the amino acid numbering of SEQ ID NO:2. Thus, for example, in some cases, the variant IL-2 comprises the following amino acid sequence: APTSSSTKKT QLQLEHLLLD LQMILNGINN YKNPKLTRML TFKFYMPKKA TELKHLQCLE EELKPLEEVL NLAQSKNFHF DPRDVVISNVN VFILELKGSE TTFMCEYADE TATIVEFLNR WITFCQSIIS TLT (SEQ ID NO:7).

In some cases, the variant IL-2 comprises the following amino acid substitutions: Q74H, L80F, R81D, L85V, I86V, and 192F, based on the amino acid numbering of SEQ ID NO:2. Thus, for example, in some cases, the variant IL-2 comprises the following amino acid sequence: APTSSSTKKT QLQLEHLLLD LQMILNGINN YKNPKLTRML TFKFYMPKKA TELKHLQCLE EELKPLEEVL NLAHSKNFHF DPRDVVISNIN VFVLELKGSE TTFMCEYADE TATIVEFLNR WITFCQSIIS TLT (SEQ ID NO:8).

In some cases, the variant IL-2 comprises the following amino acid substitutions: Q74S, L80F, R81D, L85V, I86V, and I92F, based on the amino acid numbering of SEQ ID NO:2. Thus, for example, in some cases, the variant IL-2 comprises the following amino acid sequence: APTSSSTKKT QLQLEHLLLD LQMILNGINN YKNPKLTRML TFKFYMPKKA TELKHLQCLE EELKPLEEVL NLASSKNFHF DPRDVVISNIN VFVLELKGSE TTFMCEYADE TATIVEFLNR WITFCQSIIS TLT (SEQ ID NO:9).

In some cases, the variant IL-2 comprises the following amino acid substitutions: Q74N, L80F, R81D, L85V, I86V, and 192F, based on the amino acid numbering of SEQ ID NO:2. Thus, for example, in some cases, the variant IL-2 comprises the following amino acid sequence: APTSSSTKKT QLQLEHLLLD LQMILNGINN YKNPKLTRML TFKFYMPKKA TELKHLQCLE EELKPLEEVL NLANSKNFHF DPRDVVISNIN VFVLELKGSE TTFMCEYADE TATIVEFLNR WITFCQSIIS TLT (SEQ ID NO:10).

### Orthogonal IL-2 Variant That Binds IL-2R With Variant IL-2Rβ

A suitable IL-2 variant is one that binds selectively to an IL-2R comprising a variant IL-2Rβ; such a variant IL-2 is referred to as “ortho IL-2.”

An amino acid sequence of wild-type human IL-2, with the signal peptide (in bold and underlined), is as follows:

(SEQ ID NO: 1)
MYRMQLLSCIALSLALVTNSAPTSSSTKKT QLQLEHLLLD LQMILNGINN
YKNPKLTRML TFKFYMPKKA TELKHLQCLE EELKPLEEVL NLAQSKNFHL RPRDLISNIN
VIVLELKGSE TTFMCEYADE TATIVEFLNR WITFCQSIIS TLT.

In some cases, the IL-2 lacks the signal sequence. For example, in some cases, wild-type human IL-2 comprises the following amino acid sequence:

(SEQ ID NO: 2)
APTSSSTKKT QLQLEHLLLD LQMILNGINN YKNPKLTRML TFKFYMPKKA
TELKHLQCLE EELKPLEEVL NLAQSKNFHL RPRDLISNIN VIVLELKGSE TTFMCEYADE
TATIVEFLNR WITFCQSIIS TLT.

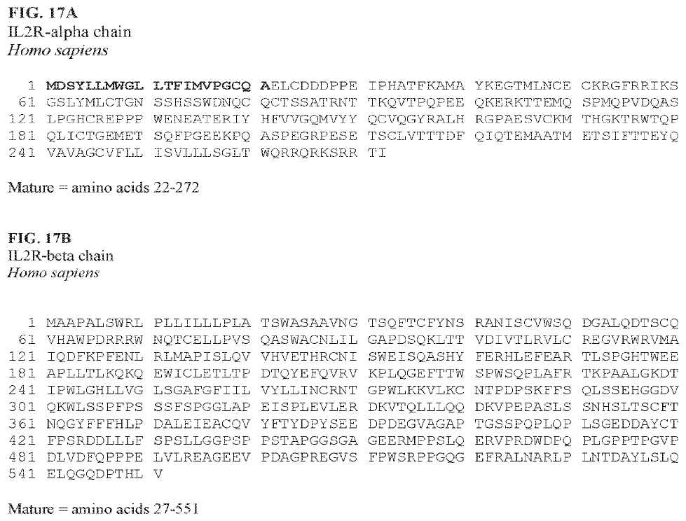

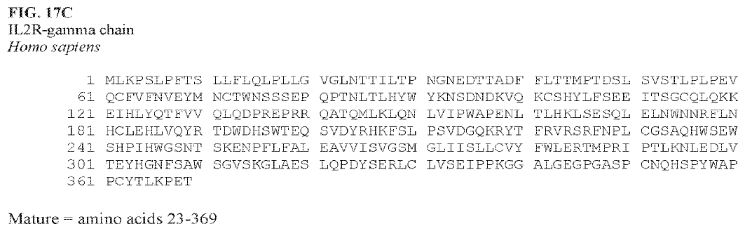

Wild-type human IL-2 binds to an IL-2 receptor (IL-2R) on the surface of a cell. An IL-2 receptor is in some cases a heterotrimeric polypeptide comprising an alpha chain (IL-2Rα; also referred to as CD25), a beta chain (IL-2Rβ; also referred to as CD122: and a gamma chain (IL-2Rγ; also referred to as CD132) Amino acid sequences of human IL-2Rα, human IL-2Rβ, and human IL-2Rγ are provided in FIG. 17A-17C.

An ortho-IL-2 polypeptide can comprise amino acid substitutions at one or more amino acids selected from Q13, L14, E15, H16, L19, D20, Q22, M23, G27, and N88, based on the amino acid numbering of SEQ ID NO:2. An ortho-IL-2 polypeptide can comprise amino acid substitutions at one or more amino acids selected from E15, H16, L19, D20, Q22, and M23, based on the amino acid numbering of SEQ ID NO:2. An ortho-IL-2 polypeptide can comprise one or more of the following amino acid substitutions: Q13W, L14M, L14W, E15D, E15T, E15A, E155, H16N, H16Q, L19V, L19I, L19A, D20L, D20M, Q22S, Q22T, Q22E, Q22K, Q22E, M23A, M23W, M23H, M23Y, M23F, M23Q, M23Y, G27K, G27S, R81D, R81Y, N88E, N88Q, and T51I. An ortho-IL-2 polypeptide can comprise H16N, L19V, D20N, Q22T, M23H, and G27K substitutions. An ortho-IL-2 polypeptide can comprise E15D, H16N, L19V, D20L, Q22T, and M23H substitutions. An ortho-IL-2 polypeptide can comprise E15D, H16N, L19V, D20L, Q22T, and M23A substitutions. An ortho-IL-2 polypeptide can comprise E15D, H16N, L19V, D20L, Q22K, M23A substitutions.

In some cases, an ortho-IL-2 polypeptide comprises one of the following sets of substitutions: [E15S; H16Q; L19V, D20T/S; Q22K, M23L/S]; [E15S; H16Q; L19I; D20S; Q22K; M23L]; [E15S; L19V; D20M; Q22K; M23S]; [E15T; H16Q; L19V; D20S; M23S]; [E15Q; L19V; D20M; Q22K; M23S]; [E15Q; H16Q; L19V; D20T; Q22K; M23V]; [E15H; H16Q; L19I; D20S; Q22K; M23L]; [E15H; H16Q; L19I; D20L; Q22K; M23T]; [19V; D20M; Q22N; M23S]; [E15S, H16Q, L19V, D20L, M23Q, R81D, T51I], [E15S, H16Q, L19V, D20L, M23Q, R81Y], [E15S, H16Q, L19V, D20L, Q22K, M23A], [E15S, H16Q, L19V, D20L, M23A].

In some cases, an ortho-IL-2 polypeptide comprises E15S5, H16Q, L19V, D20T/S/M, Q22K, and M23L/S substitutions, based on the amino acid numbering of SEQ ID NO:2. In some cases, an ortho-IL-2 polypeptide comprises E15S, H16Q, L19V, D20L, and M23Q/A substitutions, based on the amino acid numbering of SEQ ID NO:2. For example, in some cases, an ortho-IL-2 polypeptide comprises the amino acid sequence: APTSSSTKKT QLQLSQLLVT LKLILNGINN YKNPKLTRML TFKFYMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:11).

Wild-type human IL-2Rβ (mature; without signal peptide) can have the following amino acid sequence:

(SEQ ID NO: 3)
AVNG TSQFTCFYNS RANISCVWSQ DGALQDTSCQ VHAWPDRRRW NQTCELLPVS
QASWACNLIL GAPDSQKLTT VDIVTLRVLC REGVRWRVMA IQDFKPFENL RLMAPISLQV
VHVETHRCNI SWEISQASHY FERHLEFEAR TLSPGHTWEE APLLTLKQKQ EWICLETLTP
DTQYEFQVRV KPLQGEFTTW SPWSQPLAFR TKPAALGKDT IPWLGHLLVG LSGAFGFIIL
VYLLINCRNT GPWLKKVLKC NTPDPSKFFS QLSSEHGGDV QKWLSSPFPS SSFSPGGLAP
EISPLEVLER DKVTQLLLQQ DKVPEPASLS SNHSLTSCFT NQGYFFFHLP DALEIEACQV
YFTYDPYSEE DPDEGVAGAP TGSSPQPLQP LSGEDDAYCT FPSRDDLLLF SPSLLGGPSP
PSTAPGGSGA GEERMPPSLQ ERVPRDWDPQ PLGPPTPGVP DLVDFQPPPE LVLREAGEEV
PDAGPREGVS FPWSRPPGQG EFRALNARLP LNTDAYLSLQ ELQGQDPTHL V

In some cases, a variant IL-2Rβ (a variant to which an ortho-IL-2 polypeptide binds) comprises an amino acid substitution at one or more amino acids selected from R41, R42, Q70, K71, T73, T74, V75, S132, H133, Y134, F135, E136, and Q214, based on the amino acid numbering of SEQ ID NO:3. In some cases, a variant IL-2Rβ (a variant to which an ortho-IL-2 polypeptide binds) comprises an amino acid substitution at one or more amino acids selected from Q70, T73, H133, and Y134, based on the amino acid numbering of SEQ ID NO:3.

In some cases, a variant IL-2Rβ (a variant to which an ortho-IL-2 polypeptide binds) comprises one or more amino acid substitutions selected from Q70Y, T73D, T73Y, H133D, H133E, H133K, Y134F, Y134E, and Y134R, based on the amino acid numbering of SEQ ID NO:3.

As one example, a variant IL-2Rβ (a variant to which an ortho-IL-2 polypeptide binds) has the following amino acid sequence (with Q70Y, T73D, H133D, and Y134F substitutions, shown in bold, compared to SEQ ID NO:3):

(SEQ ID NO: 12)
AVNG TSQFTCFYNS RANISCVWSQ DGALQDTSCQ VHAWPDRRRW NQTCELLPVS
QASWACNLIL GAPDSYKLDT VDIVTLRVLC REGVRWRVMA IQDFKPFENL RLMAPISLQV
VHVETHRCNI SWEISQASDF FERHLEFEAR TLSPGHTWEE APLLTLKQKQ EWICLETLTP
DTQYEFQVRV KPLQGEFTTW SPWSQPLAFR TKPAALGKDT IPWLGHLLVG LSGAFGFIIL
VYLLINCRNT GPWLKKVLKC NTPDPSKFFS QLSSEHGGDV QKWLSSPFPS SSFSPGGLAP
EISPLEVLER DKVTQLLLQQ DKVPEPASLS SNHSLTSCFT NQGYFFFHLP DALEIEACQV
YFTYDPYSEE DPDEGVAGAP TGSSPQPLQP LSGEDDAYCT FPSRDDLLLF SPSLLGGPSP
PSTAPGGSGA GEERMPPSLQ ERVPRDWDPQ PLGPPTPGVP DLVDFQPPPE LVLREAGEEV
PDAGPREGVS FPWSRPPGQG EFRALNARLP LNTDAYLSLQ ELQGQDPTHL V.

### Chimeric Polypeptide

As noted above, a genetically modified, in vitro immune cell of the present disclosure is genetically modified with a nucleic acid comprising a nucleotide sequence encoding a chimeric polypeptide comprising: i) an antibody specific for a target antigen; and ii) a binding triggered transcriptional activator. In some cases, the chimeric polypeptide is a chimeric Notch polypeptide (“synNotch”). Chimeric Notch polypeptides are described in, e.g., WO 2016/138034; and U.S. Pat. No. 9,670,281. In some cases the chimeric polypeptide is a force sensor cleavage domain-containing chimeric polypeptide (also referred to herein as an “A2 chimeric polypeptide”). Force sensor cleavage domain-containing chimeric polypeptides are described in, e.g., WO 2019/099689.

### Chimeric Notch Polypeptides

In some cases, the chimeric polypeptide is a chimeric Notch polypeptide (a “synNotch” polypeptide). In some cases, the chimeric polypeptide comprises, from N-terminal to C-terminal and in covalent linkage: i) an extracellular domain comprising an antibody specific for a target antigen; ii) a Notch regulatory polypeptide (also referred to as a “Notch receptor polypeptide”) that comprises one or more proteolytic cleavage sites; and; iii) an intracellular domain comprising a transcriptional activator. Binding of the antibody to the target antigen (e.g., a target antigen present on the surface of a cell) induces cleavage of the Notch receptor polypeptide at the one or more proteolytic cleavage sites, thereby releasing the intracellular domain.

In some cases, the Notch regulatory polypeptide has a length of from 300 amino acids to 400 amino acids. In some cases, the one or more binding-inducible proteolytic cleavage sites are selected from S1, S2, and S3 proteolytic cleavage sites. In some cases, the S1 proteolytic cleavage site is a furin-like protease cleavage site comprising the amino acid sequence Arg-X-(Arg/Lys)-Arg, where X is any amino acid. In some cases, the S2 proteolytic cleavage site ADAM-17-type protease cleavage site comprising an Ala-Val dipeptide sequence. In some cases, the S3 proteolytic cleavage site is a γ-secretase cleavage site comprising a Gly-Val dipeptide sequence. In some cases, the Notch regulatory polypeptide comprises a Lin 12-Notch repeat, a heterodimerization domain comprising an S2 proteolytic cleavage site and a transmembrane domain comprising an S3 proteolytic cleavage site. In some cases, the Notch regulatory polypeptide further comprises, at its N-terminus, one or more epidermal growth factor (EGF) repeats. In some cases, the chimeric polypeptide comprises a linker interposed between the extracellular domain and the Notch regulatory polypeptide. In some cases, the target antigen is a cancer-associated antigen (e.g., a tumor-specific antigen), a disease-associated antigen, a pathogen-associated antigen, an autoimmune disease-associated antigen, or an extracellular matrix component. In some cases, the antibody is a single-chain Fv. In some cases, the antibody is a nanobody, a single-domain antibody, a diabody, a triabody, or a minibody.

### Extracellular Domain

As noted above, a chimeric Notch receptor polypeptide comprises an extracellular domain comprising an antibody specific for a target antigen. In some cases, the target antigen is present on the surface of a target cell.

The antibody can be any antigen-binding antibody-based polypeptide, a wide variety of which are known in the art. In some instances, the antibody is a single chain Fv (scFv). Other antibody based recognition domains (cAb VHH (camelid antibody variable domains) and humanized versions, IgNAR VH (shark antibody variable domains) and humanized versions, sdAb VH (single domain antibody variable domains) and “camelized” antibody variable domains are suitable for use. In some cases, the antibody is a nanobody, a single-domain antibody, a diabody, a triabody, or a minibody.

In some cases, the antigen-binding domain is specific for an epitope present in an antigen that is expressed by (synthesized by) a cancer cell, i.e., a cancer cell associated antigen. The cancer cell associated antigen can be an antigen associated with, e.g., a breast cancer cell, a B cell lymphoma, a pancreatic cancer, a Hodgkin lymphoma cell, an ovarian cancer cell, a prostate cancer cell, a mesothelioma, a lung cancer cell (e.g., a small cell lung cancer cell), a non-Hodgkin B-cell lymphoma (B-NHL) cell, an ovarian cancer cell, a prostate cancer cell, a mesothelioma cell, a lung cancer cell (e.g., a small cell lung cancer cell), a melanoma cell, a chronic lymphocytic leukemia cell, an acute lymphocytic leukemia cell, a neuroblastoma cell, a glioma, a glioblastoma, a medulloblastoma, a colorectal cancer cell, etc. A cancer cell associated antigen may also be expressed by a non-cancerous cell.

In some cases, the antigen-binding domain is specific for an epitope present in a tissue-specific antigen. In some cases, the antigen-binding domain is specific for an epitope present in a disease-associated antigen.

Non-limiting examples of antigens to which an antigen-binding domain of a subject chimeric Notch receptor polypeptide can bind include, e.g., CD19, CD20, CD38, CD30, Her2/neu, ERBB2, CA125, MUC-1, prostate-specific membrane antigen (PSMA), CD44 surface adhesion molecule, mesothelin, carcinoembryonic antigen (CEA), epidermal growth factor receptor (EGFR), EGFRvIII, vascular endothelial growth factor receptor-2 (VEGFR2), high molecular weight-melanoma associated antigen (HMW-MAA), MAGE-A1, IL-13R-a2, GD2, and the like.

Non-limiting examples of antigens to which an antigen-binding domain of a subject chimeric Notch receptor polypeptide can bind include, e.g., Cadherins (CDH1-20), Integrins (alfa and beta isoforms), Ephrins, NCAMs, connexins, CD44, syndecan, CD47, DGalfa/beta, SV2, protocadherin, Fas, Dectin-1, CD7, CD40, Neuregulin, KIR, BTLA, Tim-2, Lag-3, CD19, CTLA4, CD28, TIGIT, and ICOS.

In some cases, the antibody is specific for a cytokine. In some cases, the antibody is specific for a cytokine receptor. In some cases, the antibody is specific for a growth factor. In some cases, the antibody is specific for a growth factor receptor. In some cases, the antibody is specific for a cell-surface receptor.

In some cases, the antibody is specific for a cell surface target, where non-limiting examples of cell surface targets include CD19, CD30, Her2, CD22, ENPP3, EGFR, CD20, CD52, CD 11a, and alpha-integrin.

Where the antibody is specific for a cancer-associated antigen, the antigen can be a cancer-associated antigen, where cancer-associated antigens include, e.g., CD19, CD20, CD38, CD30, Her2/neu, ERBB2, CA125, MUC-1, prostate-specific membrane antigen (PSMA), CD44 surface adhesion molecule, mesothelin, carcinoembryonic antigen (CEA), epidermal growth factor receptor (EGFR), EGFRvIII, vascular endothelial growth factor receptor-2 (VEGFR2), high molecular weight-melanoma associated antigen (HMW-MAA), MAGE-A1, IL-13R-a2, GD2, and the like. Cancer-associated antigens also include, e.g., 4-1BB, 5T4, adenocarcinoma antigen, alpha-fetoprotein, BAFF, B-lymphoma cell, C242 antigen, CA-125, carbonic anhydrase 9 (CA-IX), C-MET, CCR4, CD152, CD19, CD20, CD200, CD22, CD221, CD23 (IgE receptor), CD28, CD30 (TNFRSF8), CD33, CD4, CD40, CD44 v6, CD51, CD52, CD56, CD74, CD80, CEA, CNTO888, CTLA-4, DRS, EGFR, EpCAM, CD3, FAP, fibronectin extra domain-B, folate receptor 1, GD2, GD3 ganglioside, glycoprotein 75, GPNMB, HER2/neu, HGF, human scatter factor receptor kinase, IGF-1 receptor, IGF-I, IgG1, L1-CAM, IL-13, IL-6, insulin-like growth factor I receptor, integrin α5β1, integrin αvβ3, MORAb-009, MS4A1, MUCl, mucin CanAg, N-glycolylneuraminic acid, NPC-1C, PDGF-R α, PDL192, phosphatidylserine, prostatic carcinoma cells, RANKL, RON, ROR1, SCH 900105, SDC1, SLAMF7, TAG-72, tenascin C, TGF beta 2, TGF-β, TRAIL-R1, TRAIL-R2, tumor antigen CTAA16.88, VEGF-A, VEGFR-1, VEGFR2, and vimentin.

The antigen can be associated with an inflammatory disease. Non-limiting examples of antigens associated with inflammatory disease include, e.g., AOC3 (VAP-1), CAM-3001, CCL11 (eotaxin-1), CD125, CD147 (basigin), CD154 (CD40L), CD2, CD20, CD23 (IgE receptor), CD25 (αchain of IL-2 receptor), CD3, CD4, CDS, IFN-α, IFN-γ, IgE, IgE Fc region, IL-1, IL-12, IL-23, IL-13, IL-17, IL-17A, IL-22, IL-4, IL-5, IL-5, IL-6, IL-6 receptor, integrin α4, integrin α4β7, LFA-1 (CD11α), myostatin, OX-40, scleroscin, SOST, TGF beta 1, TNF-α, and VEGF-A.

### Notch Regulatory Polypeptide

In some cases, the Notch receptor polypeptide (also referred to herein as a “Notch regulatory polypeptide”) present in a chimeric Notch receptor polypeptide has a length of from 50 amino acids (aa) to 1000 aa, e.g., from 50 aa to 75 aa, from 75 aa to 100 aa, from 100 aa to 150 aa, from 150 aa to 200 aa, from 200 aa to 250 aa, from 250 a to 300 aa, from 300 aa to 350 aa, from 350 aa to 400 aa, from 400 aa to 450 aa, from 450 aa to 500 aa, from 500 aa to 550 aa, from 550 aa to 600 aa, from 600 aa to 650 aa, from 650 aa to 700 aa, from 700 aa to 750 aa, from 750 aa to 800 aa, from 800 aa to 850 aa, from 850 aa to 900 aa, from 900 aa to 950 aa, or from 950 aa to 1000 aa. In some cases, the Notch receptor polypeptide present in a chimeric Notch receptor polypeptide has a length of from 300 aa to 400 aa. In some cases, the Notch receptor polypeptide present in a chimeric Notch receptor polypeptide has a length of from 300 aa to 350 aa. In some cases, the Notch receptor polypeptide present in a chimeric Notch receptor polypeptide has a length of from 300 aa to 325 aa. In some cases, the Notch receptor polypeptide present in a chimeric Notch receptor polypeptide has a length of from 350 aa to 400 aa. In some cases, the Notch receptor polypeptide present in a chimeric Notch receptor polypeptide has a length of from 750 aa to 850 aa. In some cases, the Notch receptor polypeptide present in a chimeric Notch receptor polypeptide of the present disclosure has a length of from 50 aa to 75 aa. In some cases, the Notch receptor polypeptide present in a chimeric Notch receptor polypeptide has a length of from 310 aa to 320 aa, e.g., 310 aa, 311 aa, 312 aa, 313 aa, 314 aa, 315 aa, 316 aa, 317 aa, 318 aa, 319 aa, or 320 aa. In some cases, the Notch receptor polypeptide present in a chimeric Notch receptor polypeptide has a length of 315 aa. In some cases, the Notch receptor polypeptide present in a chimeric Notch receptor polypeptide has a length of from 360 aa to 370 aa, e.g., 360 aa, 361 aa, 362 aa, 363 aa 364 aa, 365 aa, 366 aa, 367 aa, 368 aa, 369 aa, or 370 aa. In some cases, the Notch receptor polypeptide present in a chimeric Notch receptor polypeptide has a length of 367 aa.

In some cases, the Notch receptor polypeptide present in a chimeric Notch receptor polypeptide comprises an amino acid sequence having at least 75%, at least 80%, at least 85%, at least 90%, at least 95%, at least 98%, at least 99%, or 100%, amino acid sequence identity to the following amino acid sequence:

(SEQ ID NO: 13)
IPYKIEAVKSEPVEPPLPSQLHLMYVAAAAFVLLFFVGCGVLLSRKRRR
QLCIQKL;

where the transmembrane™ domain is underlined; where the Notch receptor polypeptide comprises an S2 proteolytic cleavage site and an S3 proteolytic cleavage site; where the Notch receptor polypeptide has a length of from 50 amino acids (aa) to 65 aa, e.g., 50, 51, 52, 53, 54, 55, 56, 57, 58, 59, 60, 61, 62, 63, 64, or 65 aa. In some cases, the Notch receptor polypeptide present in a chimeric Notch receptor polypeptide comprises an amino acid sequence having at least 75%, at least 80%, at least 85%, at least 90%, at least 95%, at least 98%, at least 99%, or 100%, amino acid sequence identity to the following amino acid sequence:

(SEQ ID NO: 13)
IPYKIEAVKSEPVEPPLPSQLHLMYVAAAAFVLLFFVGCGVLLSRKRRR
QLCIQKL;

where the TM domain is underlined; where the Notch receptor polypeptide comprises an S2 proteolytic cleavage site and an S3 proteolytic cleavage site; where the Notch receptor polypeptide has a length of 56 amino acids.

In some cases, the Notch regulatory polypeptide present in a chimeric Notch receptor polypeptide of the present disclosure comprises, in order from N-terminus to C-terminus: i) a LNR-A segment; ii) a LNR-B segment; iii) a LNR-C segment; iv) an HD-N segment, v) an HD-C segment; and vi) a TM domain A LNR-A segment, LNR-B segment, and LNR-C segment can collectively be referred to as an “LNR segment.”

An LNR segment can comprise an amino acid sequence having at least 75%, at least 80%, at least 85%, at least 90%, at least 95%, at least 98%, at least 99%, or 100%, amino acid sequence identity to the following amino acid sequence:

PPQIEEACELPECQVDAGNKVCNLQCNNHACGWDGGDCSLNFNDPWKNCTQSLQCWKYFSDG HCDSQCNSAGCLFDGFDCQLTEGQCNPLYDQYCKDHFSDGHCDQGCNSAECEWDGLDC (SEQ ID NO:15); and can have a length of from 118 to 122 amino acids (e.g., 118, 119, 120, 121, or 122 amino acids).

An HD segment (HD-N plus HD-C) can comprise an amino acid sequence having at least 75%, at least 80%, at least 85%, at least 90%, at least 95%, at least 98%, at least 99%, or 100%, amino acid sequence identity to the following amino acid sequence:

AAGTLVLVVLLPPDQLRNNSFHFLRELSHVLHTNVVFKRDAQGQQMIFPYYGHEEELRKHPIKR STVGWATSSLLPGTSGGRQRRELDPMDIRGSIVYLEIDNRQCVQSSSQCFQSATDVAAFLGALAS LGSLNIPYKIEAVKSEPVEPPLP (SEQ ID NO:16); and can have a length of 150, 151, 152, 153, or 154 amino acids.

A transmembrane segment can comprise an amino acid sequence having at least 75%, at least 80%, at least 85%, at least 90%, at least 95%, at least 98%, at least 99%, or 100%, amino acid sequence identity to the following amino acid sequence: HLMYVAAAAFVLLFFVGCGVLLS (SEQ ID NO:17); and can have a length of 21, 22, 23, 24, or 25 amino acids.

In some cases, the Notch regulatory polypeptide comprises an amino acid sequence having at least 75%, at least 80%, at least 85%, at least 90%, at least 95%, at least 98%, or 100% amino acid sequence identity to the following amino acid sequence:

(SEQ ID NO: 18)
PPQIEEACELPECQVDAGNKVCNLQCNNHACGWDGGDCSLNFNDPWKNCTQ
SLQCWKYFSDGHCDSQCNSAGCLFDGFDCQLTEGQCNPLYDQYCKDHFSDG
HCDQGCNSAECEWDGLDCAEHVPERLAAGTLVLVVLLPPDQLRNNSFHFLR
ELSHVLHTNVVFKRDAQGQQMIFPYYGHEEELRKHPIKRSTVGWATSSLLP
GTSGGRQRRELDPMDIRGSIVYLEIDNRQCVQSSSQCFQSATDVAAFLGAL
ASLGSLNIPYKIEAVKSEPVEPPLPSQLHLMYVAAAAFVLLFFVGCGVLL
S.

In some cases, the Notch receptor polypeptide comprises an amino acid sequence having at least 75%, at least 80%, at least 85%, at least 90%, at least 95%, at least 98%, or 100% amino acid sequence identity to the following sequence:

PCVGSNPCYNQGTCEPTSENPFYRCLCPAKFNGLLCHILDYSFTGGAGRDIPPPQIEEACELPECQ VDAGNKVCNLQCNNHACGWDGGDCSLNFNDPWKNCTQSLQCWKYFSDGHCDSQCNSAGCLF DGFDCQLTEGQCNPLYDQYCKDHFSDGHCDQGCNSAECEWDGLDCAEHVPERLAAGTLVLVV LLPPDQLRNNSFHFLRELSHVLHTNVVFKRDAQGQQMIFPYYGHEEELRKHPIKRSTVGWATSS LLPGTSGGRQRRELDPMDIRGSIVYLEIDNRQCVQSSSQCFQSATDVAAFLGALASLGSLNIPYKI EAVKSEPVEPPLPSQLHLMYVAAAAFVLLFFVGCGVLLS (SEQ ID NO:19). In some cases, the one or more ligand-inducible proteolytic cleavage sites are selected from S1, S2, and S3 proteolytic cleavage sites. In some cases, the 51 proteolytic cleavage site is a furin-like protease cleavage site comprising the amino acid sequence Arg-X-(Arg/Lys)-Arg, where X is any amino acid. In some cases, the S2 proteolytic cleavage site ADAM-17-type protease cleavage site comprising an Ala-Val dipeptide sequence. In some cases, the S3 proteolytic cleavage site is a γ-secretase cleavage site comprising a Gly-Val dipeptide sequence.

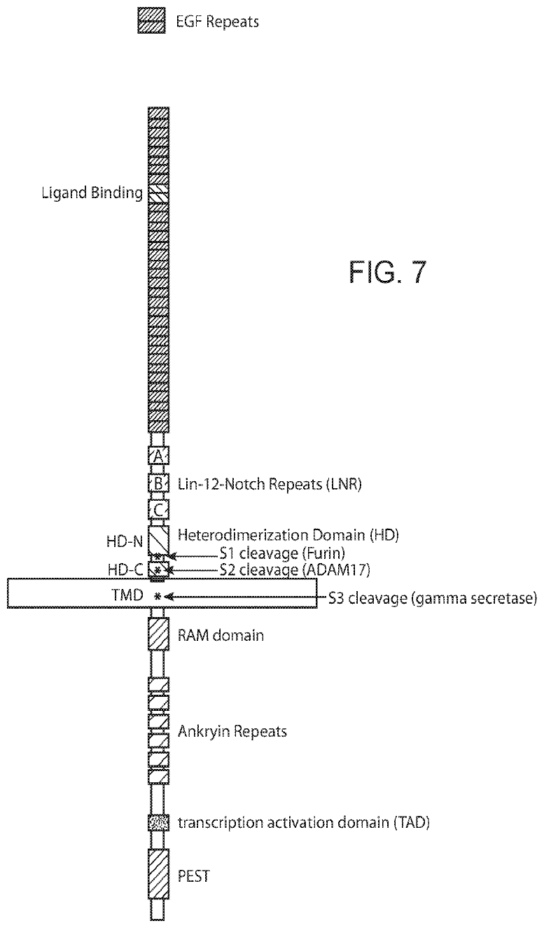

A schematic depiction of a Notch receptor polypeptide is provided in FIG. 7. The Notch receptor polypeptide depicted in FIG. 7 includes: a) an extracellular portion that includes: i) epidermal growth factor (EGF) repeats; ii) a ligand binding site; iii) three Lin-12 Notch repeats (LNR), designated LNR-A, LNR-B, and LNR-C; iv) two heterodimerization domains (HD-N and HD-C); b) a transmembrane (TM) portion; and c) an intracellular portion that includes: i) a RAM domain; ii) ankyrin repeats; iii) a transcription activation domain; and iv) a PEST region. A Notch receptor polypeptide includes three proteolytic sites, termed 51, S2, and S3. 51, a furin cleavage site, is located between HD-N and HC-C; S2, an ADAM17 cleavage site, is located within HD-C; and S3, a gamma secretase cleavage site, is within the TM portion. A Notch receptor polypeptide mediates cell-to-cell communication, e.g. communication between contacting cells, in which one contacting cell is a “receiver” cell and the other contacting cell is a “sender” cell. Engagement of a Notch receptor polypeptide present on a receiving cell by a Delta polypeptide (“ligand”) present on a sending cell results in ligand-induced cleavage of the Notch receptor polypeptide, resulting in release of the intracellular portion of the receptor from the membrane into the cytoplasm. The released portion alters receiver cell behavior by functioning as a transcriptional regulator.

In some cases, the Notch receptor polypeptide present in a chimeric Notch receptor polypeptide of the present disclosure comprises, in order from N-terminus to C-terminus: i) a single EGF repeat; ii) an LNR segment; iii) an HD-N segment, iv) an HD-C segment; and v) a TM domain

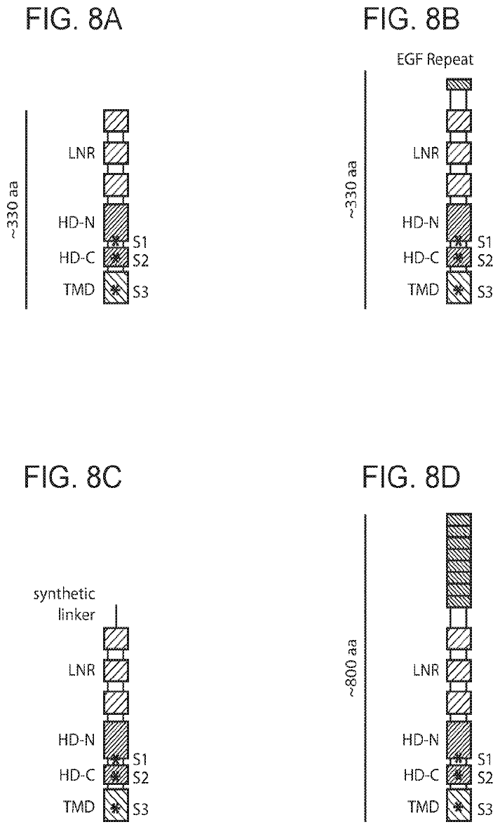

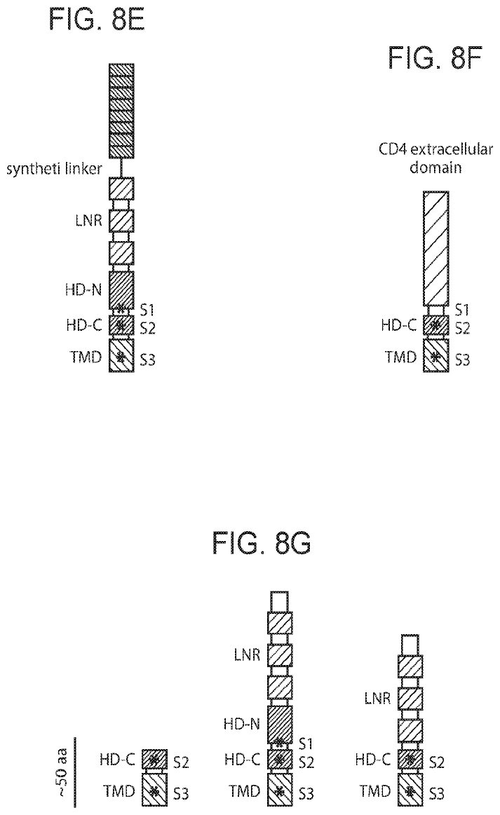

In some cases, the Notch receptor polypeptide present in a chimeric Notch receptor polypeptide of the present disclosure comprises, in order from N-terminus to C-terminus: i) a LNR-A segment; ii) a LNR-B segment; iii) a LNR-C segment; iv) an HD-N segment, v) an HD-C segment; and vi) a TM domain An LNR-A segment, LNR-B segment, and LNR-C segment can collectively be referred to as an “LNR segment.” Such a Notch receptor polypeptide is depicted schematically in FIG. 8A.

In some cases, the Notch receptor polypeptide present in a chimeric Notch receptor polypeptide of the present disclosure comprises, in order from N-terminus to C-terminus: i) a single EGF repeat; ii) an LNR segment; iii) an HD-N segment, iv) an HD-C segment; and v) a TM domain Such a Notch receptor polypeptide is depicted schematically in FIG. 8B

In some cases, a Notch receptor polypeptide comprises a synthetic linker. For example, in some cases, a Notch receptor polypeptide comprises, in order from N-terminus to C-terminus: i) a synthetic linker; ii) an EGF repeat; iii) an LNR segment; iv) an HD-N segment, v) an HD-C segment; and vi) a TM domain Such a Notch receptor polypeptide is depicted schematically in FIG. 8C.

In some cases, the Notch receptor polypeptide present in a chimeric Notch receptor polypeptide of the present disclosure comprises, in order from N-terminus to C-terminus: i) from two to eleven EGF repeats; ii) an LNR segment; iii) an HD-N segment, iv) an HD-C segment; and v) a TM domain Such a Notch receptor polypeptide is depicted schematically in FIG. 8D.

In some cases, a Notch receptor polypeptide comprises a synthetic linker. For example, in some cases, a Notch receptor polypeptide comprises, in order from N-terminus to C-terminus: i) two to eleven EGF repeats; ii) a synthetic linker; iii) an LNR segment; iv) an HD-N segment, v) an HD-C segment; and vi) a TM domain. Such a Notch receptor polypeptide is depicted schematically in FIG. 8E.

A synthetic linker can have a length of from about 10 amino acids (aa) to about 200 aa, e.g., from 10 aa to 25 aa, from 25 aa to 50 aa, from 50 aa to 75 aa, from 75 aa to 100 aa, from 100 aa to 125 aa, from 125 aa to 150 aa, from 150 aa to 175 aa, or from 175 aa to 200 aa. A synthetic linker can have a length of from 10 aa to 30 aa, e.g., 10, 11, 12, 13, 14, 15, 16, 17, 18, 19, 20, 21, 22, 23, 24, 25, 26, 27, 28, 29, or 30 aa. A synthetic linker can have a length of from 30 aa to 50 aa, e.g., from 30 aa to 35 aa, from 35 aa to 40 aa, from 40 aa to 45 aa, or from 45 aa to 50 aa.

In some cases, a Notch receptor polypeptide comprises, in order from N-terminus to C-terminus: i) an HD-C segment; and ii) a TM domain, where the Notch receptor polypeptide does not include an LNR segment. In some cases, the LNR segment is replaced with a heterologous polypeptide. Such a Notch receptor polypeptide is depicted schematically in FIG. 8F.

In some cases, the Notch receptor polypeptide lacks an S1 ligand-inducible proteolytic cleavage site. In some cases, the Notch receptor polypeptide lacks an S2 ligand-inducible proteolytic cleavage site. In some cases, the Notch receptor polypeptide lacks an S3 ligand-inducible proteolytic cleavage site. In some cases, the Notch receptor polypeptide lacks both an S1 ligand-inducible proteolytic cleavage site and an S2 ligand-inducible proteolytic cleavage site. In some cases, the Notch receptor polypeptide includes an S3 ligand-inducible proteolytic cleavage site; and lacks both an S1 ligand-inducible proteolytic cleavage site and an S2 ligand-inducible proteolytic cleavage site. Examples are depicted schematically in FIG. 8G.

An EGF repeat can comprise an amino acid sequence having at least 75%, at least 80%, at least 85%, at least 90%, at least 95%, at least 98%, at least 99%, or 100%, amino acid sequence identity to the following sequence: PCVGSNPCYNQGTCEPTSENPFYRCLCPAKFNGLLCH (SEQ ID NO:20); and can have a length of 35 amino acids to 40 amino acids (e.g., 35, 36, 37, 38, 39, or 40 amino acids. An EGF repeat can comprise an amino acid sequence having at least 75%, at least 80%, at least 85%, at least 90%, at least 95%, at least 98%, at least 99%, or 100%, amino acid sequence identity to the amino acid sequence DINECVLSPCRHGASCQNTHGGYRCHCQAGYSGRNCE (SEQ ID NO:21); and can have a length of from 35 amino acids to about 40 amino acids (aa) (e.g., 35, 36, 37, 38, 39, or 40 aa). An EGF repeat can comprise an amino acid sequence having at least 75%, at least 80%, at least 85%, at least 90%, at least 95%, at least 98%, at least 99%, or 100%, amino acid sequence identity to the amino acid sequence DIDDCRPNPCHNGGSCTDGINTAFCDCLPGFRGTFCE (SEQ ID NO:22); and can have a length of from 35 amino acids to about 40 amino acids (aa) (e.g., 35, 36, 37, 38, 39, or 40 aa). Other suitable EGF repeat sequences include an amino acid sequence having at least 75%, at least 80%, at least 85%, at least 90%, at least 95%, at least 98%, at least 99%, or 100%, amino acid sequence identity to: i) DVNECDSQPCLHGGTCQDGCGSYRCTCPQGYTGPNCQ (SEQ ID NO:23) (where the EGF repeat has a length of from 35 amino acids to about 40 amino acids (aa) (e.g., 35, 36, 37, 38, 39, or 40 aa)); ii) LVDECSPSPCQNGATCTDYLGGYSCKCVAGYHGVNC (SEQ ID NO:24) (where the EGF repeat has a length of from 35 amino acids to about 40 amino acids (aa) (e.g., 35, 36, 37, 38, 39, or 40 aa)); iii) IDECLSHPCQNGGTCLDLPNTYKCSCPRGTQGVHCE (SEQ ID NO:25) (where the EGF repeat has a length of from 35 amino acids to about 40 amino acids (aa) (e.g., 35, 36, 37, 38, 39, or 40 aa)); and iv) CFNNGTCVDQVGGYSCTCPPGFVGERC (SEQ ID NO:26) (where the EGF repeat has a length of from 25 amino acids (aa) to 30 aa, e.g., 25, 26, 27, 28, 29, or 30 aa).

In some cases, the Notch receptor polypeptide present in a chimeric Notch receptor polypeptide of the present disclosure comprises, in order from N-terminus to C-terminus: i) two EGF repeats; ii) an LNR segment; iii) an HD-N segment, iv) an HD-C segment; and v) a TM domain In some cases, the Notch receptor polypeptide present in a chimeric Notch receptor polypeptide of the present disclosure comprises, in order from N-terminus to C-terminus: i) three EGF repeats; ii) an LNR segment; iii) an HD-N segment, iv) an HD-C segment; and v) a TM domain In some cases, the Notch receptor polypeptide present in a chimeric Notch receptor polypeptide of the present disclosure comprises, in order from N-terminus to C-terminus: i) four EGF repeats; ii) an LNR segment; iii) an HD-N segment, iv) an HD-C segment; and v) a TM domain In some cases, the Notch receptor polypeptide present in a chimeric Notch receptor polypeptide of the present disclosure comprises, in order from N-terminus to C-terminus: i) five EGF repeats; ii) an LNR segment; iii) an HD-N segment, iv) an HD-C segment; and v) a TM domain In some cases, the Notch receptor polypeptide present in a chimeric Notch receptor polypeptide of the present disclosure comprises, in order from N-terminus to C-terminus: i) six EGF repeats; ii) an LNR segment; iii) an HD-N segment, iv) an HD-C segment; and v) a TM domain In some cases, the Notch receptor polypeptide present in a chimeric Notch receptor polypeptide of the present disclosure comprises, in order from N-terminus to C-terminus: i) seven EGF repeats; ii) an LNR segment; iii) an HD-N segment, iv) an HD-C segment; and v) a TM domain In some cases, the Notch receptor polypeptide present in a chimeric Notch receptor polypeptide of the present disclosure comprises, in order from N-terminus to C-terminus: i) eight EGF repeats; ii) an LNR segment; iii) an HD-N segment, iv) an HD-C segment; and v) a TM domain. In some cases, the Notch receptor polypeptide present in a chimeric Notch receptor polypeptide of the present disclosure comprises, in order from N-terminus to C-terminus: i) nine EGF repeats; ii) an LNR segment; iii) an HD-N segment, iv) an HD-C segment; and v) a TM domain In some cases, the Notch receptor polypeptide present in a chimeric Notch receptor polypeptide of the present disclosure comprises, in order from N-terminus to C-terminus: i) ten EGF repeats; ii) an LNR segment; iii) an HD-N segment, iv) an HD-C segment; and v) a TM domain In some cases, the Notch receptor polypeptide present in a chimeric Notch receptor polypeptide of the present disclosure comprises, in order from N-terminus to C-terminus: i) eleven EGF repeats; ii) an LNR segment; iii) an HD-N segment, iv) an HD-C segment; and v) a TM domain

In some cases, the Notch receptor polypeptide includes only one binding-inducible proteolytic cleavage site. In some cases, the Notch receptor polypeptide includes two binding-inducible proteolytic cleavage sites. In some cases, the Notch receptor polypeptide includes three binding-inducible proteolytic cleavage sites. For simplicity, binding-inducible cleavage sites will be referred to herein as “S1,” “S2,” and “S3” binding-inducible proteolytic cleavage sites.

In some cases, the Notch receptor polypeptide includes an S1 ligand-inducible proteolytic cleavage site. An S1 ligand-inducible proteolytic cleavage site can be located between the HD-N segment and the HD-C segment. In some cases, the S1 ligand-inducible proteolytic cleavage site is a furin-like protease cleavage site. A furin-like protease cleavage site can have the canonical sequence Arg-X-(Arg/Lys)-Arg, where X is any amino acid; the protease cleaves immediately C-terminal to the canonical sequence. For example, in some cases, an amino acid sequence comprising an S1 ligand-inducible proteolytic cleavage site can have the amino acid sequence GRRRRELDPM (SEQ ID NO:27), where cleavage occurs between the “RE” sequence. As another example, an amino acid sequence comprising an S1 ligand-inducible proteolytic cleavage site can have the amino acid sequence RQRRELDPM (SEQ ID NO:28), where cleavage occurs between the “RE” sequence.

In some cases, the Notch receptor polypeptide includes an S2 ligand-inducible proteolytic cleavage site. An S2 ligand-inducible proteolytic cleavage site can be located within the HD-C segment. In some cases, the S2 ligand-inducible proteolytic cleavage site is an ADAM-17-type protease cleavage site. An ADAM-17-type protease cleavage site can comprise an Ala-Val dipeptide sequence, where the enzyme cleaves between the Ala and the Val. For example, in some cases, amino acid sequence comprising an S2 ligand-inducible proteolytic cleavage site can have the amino acid sequence KIEAVKSE (SEQ ID NO:29), where cleavage occurs between the “AV” sequence. As another example, an amino acid sequence comprising an S2 ligand-inducible proteolytic cleavage site can have the amino acid sequence KIEAVQSE (SEQ ID NO:30), where cleavage occurs between the “AV” sequence.

In some cases, the Notch receptor polypeptide includes an S3 ligand-inducible proteolytic cleavage site. An S3 ligand-inducible proteolytic cleavage site can be located within the TM domain In some cases, the S3 ligand-inducible proteolytic cleavage site is a gamma-secretase (γ-secretase) cleavage site. A y-secretase cleavage site can comprise a Gly-Val dipeptide sequence, where the enzyme cleaves between the Gly and the Val. For example, in some cases, an S3 ligand-inducible proteolytic cleavage site has the amino acid sequence VGCGVLLS (SEQ ID NO:31), where cleavage occurs between the “GV” sequence. In some cases, an S3 ligand-inducible proteolytic cleavage site comprises the amino acid sequence GCGVLLS (SEQ ID NO:32).

In some cases, the Notch receptor polypeptide lacks an S1 ligand-inducible proteolytic cleavage site. In some cases, the Notch receptor polypeptide lacks an S2 ligand-inducible proteolytic cleavage site. In some cases, the Notch receptor polypeptide lacks an S3 ligand-inducible proteolytic cleavage site. In some cases, the Notch receptor polypeptide lacks both an S1 ligand-inducible proteolytic cleavage site and an S2 ligand-inducible proteolytic cleavage site. In some cases, the Notch receptor polypeptide includes an S3 ligand-inducible proteolytic cleavage site; and lacks both an S1 ligand-inducible proteolytic cleavage site and an S2 ligand-inducible proteolytic cleavage site.

### Intracellular Domain

A chimeric Notch polypeptide comprises an intracellular domain comprising a binding triggered transcriptional activator. Binding of the antibody to its target antigen induces cleavage of the Notch receptor polypeptide at the one or more proteolytic cleavage sites, thereby releasing the intracellular domain. The transcriptional activator binds to a transcriptional control element that is operably linked to the nucleotide sequence encoding the cytokine or proliferation-inducing polypeptide, and activates transcription, such that the cytokine or proliferation-inducing polypeptide is produced.

Non-limiting examples of suitable transcriptional regulators include, e.g., ABT1, ACYP2, AEBP1, AEBP2, AES, AFF1, AFF3, AHR, ANK1, ANK2, ANKFY1, ANKIB1, ANKRD1, ANKRD10, ANKRD2, ANKRD32, ANKRD46, ANKRD49, ANKRD56, ANKRD57, ANKS4B, AR, ARHGAP17, ARID1A, ARID1B, ARID3A, ARID4A, ARID5B, ARNT, ARNT2, ARNTL, ARNTL2, ARX, ASB10, ASB11, ASB12, ASB15, ASB2, ASB5, ASB8, ASB9, ASH1L, ASH2L, ASXL1, ASZ1, ATF1, ATF3, ATF4, ATF4, ATF5, ATF6, ATF7, ATF7IP, ATM, ATOH1, ATXN3, 1300003B13RIK, B3GAT3, B930041F14RIK, BACH1, BACH2, BARX1, BARX2, BATF, BATF2, BATF3, BAZ2A, BBX, BC003267, BCL11A, BCL11B, BCL3, BCL6, BCL6B, BCLAF1, BCOR, BHLHA15, BHLHE40, BHLHE41, BLZFl, BMYC, BNC1, BNC2, BPNT1, BRCA1, BRWD1, BTBD11, BTF3, 6030408C04RIK, CAMK4, CARHSP1, CARM1, CBX4, CBX7, CCNC, CCNH, CCNT1, CCNT2, CDC5L, CDK2, CDK4, CDK9, CDKN2C, CDX1, CDX1, CDX2, CEBPA, CEBPB, CEBPD, CEBPG, CEBPG, CEBPZ, CHD4, CHD7, CHGB, CIC, CIITA, CITED1, CITED2, CITED4, CLOCK, CLPB, CML3, CNOT7, COPS2, CREB1, CREB3, CREB3L1, CREB3L1, CREB3L2, CREB3L3, CREB5, CREBBP, CREBL2, CREM, CSDA, CSDA, CSDC2, CSDE1, CTBP2, CTCF, CTCFL, CTNNB1, CTNNBL1, CXXCl, D11BWG0517E, 2300002D11RIK, DACH1, DAXX, DBP, DDIT3, DDX20, DDX54, DDX58, DEAF1, DEK, DIDO1, DLX2, DMRT1, DMRT2, DMRTB1, DNMT1, DNMT3A, DR1, DRG1, DUSP26, DYSFIP1, E2F1, E2F2, E2F3, E2F5, E2F6, EBF1, EBF2, EBF3, EBF3, EED, EGR1, EGR2, EGR3, EHF, EHMT2, EID2, ELAVL2,ELF1, ELF1, ELF2, ELF3, ELF4, ELF5, ELK3, ELK4, ELL2, EMX2, EMX2, EN2, ENPP2, EOMES, EP300, EPAS1, ERF, ERG, ESR1, ESRRA, ESRRB, ESRRG, ETS1, ETS2, ETV1, ETV3, ETV4, ETV5, ETV6, EVI1, EWSR1, EZH1, EZH2, FAH, FBXL10, FBXL11, FBXW7, FEM1A, FEM1B, FEM1C, FHL2, FLI1, FMNL2, FOS, FOSB, FOSL1, FOSL2, FOXA1, FOXA2, FOXA3, FOXCl, FOXD1, FOXD2, FOXD3, FOXF1, FOXF1A, FOXF2, FOXG1, FOXI1, FOXJ2, FOXJ3, FOXKl, FOXK2, FOXL1, FOXL2, FOXMl, FOXN1, FOXN2, FOXN3, FOXO1, FOXO3, FOXP1, FOXP2, FOXP3, FOXP4, FOXQ1, FUS, FUSIP1, 2810021G02RIK, GABPA, GABPB1, GARNL1, GAS7, GATA1, GATA2, GATA3, GATA4, GATA5, GATA5, GATA6, GBX2, GCDH, GCM1, GFIl, GFI1B, GLI2, GLI3, GLIS1, GLIS2, GLIS3, GLS2, GMEB1, GMEB2, GRHL1, GRHL2, GRHL3, GRLF1, GTF2A1, GTF2B, GTF2E2, GTF2F1, GTF2F2, GTF2H2, GTF2H4, GTF2I, GTF2IRD1, GTF2IRD1, GZFl, HAND2, HBP1, HCLS1, HDAC10, HDAC11, HDAC2, HDAC5, HDAC9, HELZ, HEST, HES4, HESS, HES6, HEXIM1, HEY2, HEYL, HHEX, HHEX, HIC1, HIC2, HIF1A, HIF1AN, HIPK2, HIVEP1, HIVEP2, HIVEP2, HIVEP3, HLF, HLTF, HLX, HMBOX1, HMG20A, HMGA2, HMGB2, HMGB3, HNF1B, HNF4A, HNF4G, HOMEZ, HOXA10, HOXA11, HOXA13, HOXA2, HOXA3, HOXA4, HOXA5, HOXA6, HOXA7, HOXA9, HOXB1, HOXB2, HOXB3, HOXB4, HOXB6, HOXB7, HOXB8, HOXB9, HOXC10, HOXC10, HOXC11, HOXC5, HOXC6, HOXC8, HOXC9, HOXD8, HOXD9, HR, HSBP1, HSF2BP, HTATIP2, HTATSF1, HUWE1, 5830417I10RIK, ID1, ID2, ID3, ID3, IFNAR2, IKBKB, IKBKG, IKZFl, IKZF2, IKZF3, IKZF4, IL31RA, ILF3, ING1, ING2, ING3, ING4, INSM1, INTS12, IQWD1, IRF1, IRF1, IRF2, IRF3, IRF4, IRF5, IRF6, IRF7, IRF8, IRF8, IRX1, IRX2, IRX3, IRX4, IRX5, ISL1, ISL2, ISX, ISX, IVNS1ABP, 2810021J22RIK, JARID1A, JARID1B, JARID1C, JARID1D, JDP2, JUN, JUNB, JUND, KLF1, KLF10, KLF11, KLF12, KLF13, KLF15, KLF16, KLF2, KLF3, KLF3, KLF4, KLF5, KLF6, KLF7, KLF8, KLF9, KRR1, 6330416L07RIK, L3MBTL2, LASS2, LASS4, LASS6, LBA1, LBH, LBX1, LCOR, LDB1, LDB2, LEFT, LHX1, LHX2, LHX5, LIMD1, LIN28, LMO1, LMO4, LMX1A, LSM11, LSM4, LYL1, 9030612M13RIK, 1810007M14RIK, 3632451006RIK, MAF, MAFA, MAFB, MAFF, MAFG, MAFK, MAGED1, MAP3K12, MAPK1, MAPK3, MAPK8, MAPK8IP1, MAX, MAZ, MBD2, MCM2, MCM4, MCM5, MCM6, MCMI, MECOM, MECP2, MED12, MEDS, MEF2A, MEF2B, MEF2C, MEF2D, MEIS1, MEIS1, MEIS2, MEOX2, MESP2, MIDI, MITF, MKI67IP, MKL1, MLL1, MLL3, MLLT10, MLLT3, MLX, MLXIP, MLXIPL, MNT, MNX1, MPL, MSC, MSRB2, MSX2, MTA3, MTF1, MTF2, MTPN, MXD1, MXD4, MXI1, MYB, MYBBP1A, MYBL2, MYC, MYCBP, MYCL1, MYCN, MYEF2, MYF6, MYNN, MYOCD, MYOD1, MYOG, MYST3, MYST4, MYT1L, MZFl, NAB1, NAB2, NANOG, NARG1, NCOA1, NCOA2, NCOA3, NCOR1, NCOR2, NDN, NEUROD1, NEUROD4, NEUROD6, NEUROG1, NEUROG2, NFAT5, NFATC1, NFATC2, NFATC2IP, NFATC3, NFATC3, NFATC4, NFE2, NFE2L1, NFE2L2, NFIA, NFIA, NFIB, NFIC, NFIL3, NFIX, NFKB1, NFKB2, NFKBIB, NFKBIE, NFKBIZ, NFX1, NFXL1, NFYA, NFYB, NHLH1, NKX2-2, NKX2-3, NKX2-5, NKX2-6, NKX6-2, NMI, NOTCH1, NOTCH2, NOTCH3, NOTCH4, NPAS1, NPAS2, NPAS3, NROB1, NROB2, NR1D1, NR1D2, NR1H3, NR1H4, NR1I2, NR1I3, NR2C1, NR2C2, NR2E3, NR2F1, NR2F2, NR2F6, NR3C1, NR3C2, NR4A1, NR4A2, NR4A2, NR4A3, NR5A1, NR5A2, NRARP, NRIP1, NRIP2, NSBP1, NSD1, NUDT12, NULL, NUPR1, 1700065013RIK, OLIG1, OLIG2, OLIG2, ONECUT1, ONECUT2, ONECUT3, ORC2L, OSGIN1, OSR1, OSR2, OSTF1, OVOL1, OVOL2, PAPOLA, PAPOLG, PAPPA2, PATZ1, PAWR, PAX2, PAX5, PAX6, PAX7, PAX8, PAX9, PBX1, PBX2, PBX3, PBX4, PCBD1, PCGF6, PDCD11, PDLIM4, PDX1, PEG3, PERI, PFDN1, PGR, PHF1, PHF10, PHF12, PHF13, PHF14, PHF20, PHF21A, PHF5A, PHF7, PHOX2A, PHOX2B, PIAS2, PIR, PITX1, PITX2, PKNOX1, PKNOX2, PLA2G6, PLAGL1, PLAGL2, PLRG1, PML, POGK, POLR2B, POLR2E, POLR2H, POLR3E, POLR3H, POLRMT, POU1F1, POU2AF1, POU2F1, POU2F2, POU3F2, POU3F3, POU3F3, POU5F1, POU6F1, PPARA, PPARD, PPARG, PPARGC1A, PPARGC1B, PPP1R12C, PPP1R13B, PPP1R16B, PPP1R1B, PPP2R1A, PPP3CB,PQBP1, PRDM1, PRDM14, PRDM15, PRDM16, PRDM2, PRDM4, PRDM5, PRDM6, PRDM8, PREB, PRKAR1A, PRKCBP1, PROX1, PRRX1, PRRX2, PSMC5, PSMD10, PSMD9, PTF1A, PTGES2, PURB, PWP1, RAB11A, RAB11B, RAB15, RAB18, RAB1B, RAB25, RAB8A, RAB8B, RAI14, RARA, RARB, RARG, RASSF7, RB1, RBBP7, RBL1, RBM14, RBM39, RBM9, RBPJ, RBPJL, RCOR2, REL, RELA, RELB, RERE, REST, REXO4, RFC1, RFX1, RFX2, RFX3, RFX5, RFX7, RFX8, RHOX5, RHOX6, RHOX9, RIPK4, RNF12, RNF14, RNF141, RNF38, RNF4, RORA, RORA, RORB, RORC, RPS6KA4, RREB1, RSRC1, RUNX1, RUNX1T1, RUNX2, RUNX2, RUNX3, RUVBL1, RUVBL2, RXRA, RXRG, RYBP, SAFB2, SALL1, SALL1, SALL2, SALL4, SAP30, SAP3OBP, SATB1, SATB2, SATB2, SCAND1, SCAP, SCRT2, SEC14L2, SERTAD1, SF1, SFPI1, SFRS5, SH3D19, SH3PXD2B, SHANK3, SHOX2, SHPRH, SIN3A, SIN3B, SIRT2, SIRT3, SIRT5, SIX1, SIX1, SIX2, SIX3, SIX4, SIX5, SKI, SMAD1, SMAD2, SMAD3,SMAD7, SMARCA1, SMARCA2, SMARCA5, SMARCB1, SMYD1, SNAIl, SNAI2, SNAPC2, SNAPC4, SNIP1, SOLH, SOX1, SOX10, SOX11, SOX12, SOX13, SOX15, SOX17, SOX18, SOX2, SOX21, SOX4, SOX5, SOX6, SOX7, SOX8, SOX9, SP1, SP110, SP140L, SP2, SP3, SP4, SP6, SP8, SPDEF, SPEN, SPI1, SPIB, SQSTM1, SREBF1, SREBF2, SREBF2, SRF, SSBP2, SSBP3, SSBP4, SSRP1, ST18, STAG1, STAT1, STAT1, STAT2, STAT3, STAT4, STAT5A, STAT5B, STAT5B, STATE, SUB1, SUZ12, TADA2L, TAF13, TAF5, TAF5L, TAF7, TAF9, TAL1,TAL1, TARDBP, TBPL1, TBR1, TBX1, TBX10, TBX15, TBX18, TBX2, TBX2, TBX20, TBX21, TBX3, TBX4, TBX5, TBX6, TCEA1, TCEA3, TCEAL1, TCEB3, TCERG1, TCF12, TCF15, TCF19, TCF20, TCF21, TCF21, TCF3, TCF4, TCF7, TCF7L2, TCFAP2A, TCFAP2B, TCFAP2C, TCFCP2L1, TCFE2A, TCFE3, TCFEB, TCFEC, TCFL5, TEAD1, TEAD2, TEAD3, TEAD4, TEF, TFAP2A, TFAP2C, TFCP2L1, TFDP2, TFEB, TFEC, TGFB1I1, TGIF1, TGIF2, TGIF2LX, THRA, THRAP3, THRB, THRSP, TIAL1, TLE1, TLE6, TMEM131, TMPO, TNFAIP3, TOB1, TOX4, TP63, TRERF1, TRIB3, TRIM24, TRIM28, TRIM30, TRIP13, TRIP4, TRIPE, TRP53, TRP53BP1, TRP63, TRPS1, TRPS1, TSC22D1, TSC22D2, TSC22D3, TSC22D4, TSHZ1, TSHZ1, TSHZ3, TTRAP, TUB, TULP4, TWIST1, TWIST2, TYSND1, UBE2W, UBN1, UBP1, UBTF, UGP2, UHRF1, UHRF2, UNCX, USF1, USF2, UTF1, VDR, VEZFl, VGLL2, VSX1, WASL, WHSC1, WHSC2, WT1, WWP1, WWTR1, XBP1, YAF2, YY1, ZBED1, ZBED4, ZBTB1, ZBTB10, ZBTB16, ZBTB16, ZBTB17, ZBTB2, ZBTB20, ZBTB22, ZBTB25, ZBTB32, ZBTB38, ZBTB4, ZBTB43, ZBTB45, ZBTB47, ZBTB7A, ZBTB7B, ZBTB7C, ZCCHC8, ZDHHC13, ZDHHC16, ZDHHC21, ZDHHC5, ZDHHC6, ZEB2, ANK2ZEB2, ZFHX2, ZFHX3, ZFHX4, ZFP105, ZFP110, ZFP143, ZFP148, ZFP161, ZFP192, ZFP207, ZFP219, ZFP238, ZFP263, ZFP275, ZFP277, ZFP281, ZFP287, ZFP292, ZFP35, ZFP354C, ZFP36, ZFP36L1, ZFP386, ZFP407, ZFP42, ZFP423, ZFP426,ZFP445, ZFP451, ATF5ZFP451, ZFP467, ZFP52, ZFP57, ZFP592, ZFP593, ZFP597, ZFP612, ZFP637, ZFP64, ZFP647, ZFP748, ZFP810, ZFP9, ZFP91, ZFPM1, ZFPM2, ZFX, ZHX2, ZHX3, ZIC1, ZIC2, ZIC3, ZIC4, ZIC5, ZKSCAN1, ZKSCAN3, ZMYND11, ZNF143, ZNF160, ZNF175, ZNF184, ZNF192, ZNF213, ZNF217, ZNF219, ZNF22, ZNF238, ZNF24, ZNF267, ZNF273, ZNF276, ZNF280D, ZNF281, ZNF292, ZNF311, ZNF331, ZNF335, ZNF337, ZNF33B, ZNF366, ZNF394, ZNF398, ZNF41, ZNF410, ZNF415, ZNF423, ZNF436, ZNF444, ZNF445, ZNF451, ZNF460, ZNF496, ZNF498, ZNF516, ZNF521, ZNF532, ZNF536, ZNF546, ZNF552, ZNF563, ZNF576, ZNF580, ZNF596, ZNF621, ZNF628, ZNF648, ZNF649, ZNF652, ZNF655, ZNF664, ZNF668, ZNF687, ZNF692, ZNF696, ZNF697, ZNF710, ZNF80, ZNF91, ZNF92, ZNRD1, ZSCAN10, ZSCAN16, ZSCAN20, ZSCAN21, ZXDC, and ZZZ3.

In some cases, the intracellular domain is a transcription factor. Suitable transcription factors include, e.g., ASCL1, BRN2, CDX2, CDX4, CTNNB1, EOMES, JUN, FOS, HNF4a, HOXAs (e.g., HOXA1, HOXA2, HOXA3, HOXA4, HOXA5, HOXA10, HOXA11, HOXA13), HOXBs (e.g., HOXB9), HOXCs (e.g., HOXC4, HOXC5, HOXC6, HOXC8, HOXC9, HOXC10, HOXC11, HOXC12, HOXC13), HOXDs (e.g., HOXD1, HOXD3, HOXD4, HOXD8, HOXD9, HOXD10, HOXD11, HOXD12, HOXD13), SNAIL-3, MYOD1, MYOG, NEUROD1-6 (e.g., NEUROD1, NEUROD2, NEUROD4, NEUROD6), PDX1, PU.1, SOX2, Nanog, Klf4, BCL-6, SOX9, STAT1-6, TBET, TCF, TEAD1-4 (e.g., TEAD1, TEAD2, TEAD3, TEAD4), TAF6L, CLOCK, CREB, GATA3, IRF7, MycC, NFkB, RORyt, RUNX1, SRF, TBX21, NFAT, MEF2D, and FoxP3.

In some cases, the intracellular domain is a transcription factor having a regulatory role in one or more immune cells (i.e., an immune cell regulatory transcription factor). Suitable immune cell regulatory transcription factors include, e.g., 2210012G02Rik, Akap81, App12, Arid4b, Arid5b, Ash11, Atf7, Atm, C430014K11Rik, Chd9, Dmtfl, Fos, Foxol, Foxpl, Hmboxl, Kdm5b, Klf2, Mga, M111, M113, Myst4, Pcgf6, Rev31, Scm14, Scp2, Smarca2, Ssbp2, Suhw4, Tcf7, Tfdp2, Tox, Zbtb20, Zbtb44, Zebl, Zfml, Zfpl, Zfp319, Zfp329, Zfp35, Zfp386, Zfp445, Zfp518, Zfp652, Zfp827, Zhx2, Eomes, Arnt1, Bbx, Hbp1, Jun, Mef2d, Mterfd1, Nfat5, Nfe212, Nr1d2, Phf21a, Taf4b, Trf, Zbtb25, Zfp326, Zfp451, Zfp58, Zfp672, Egr2, Ikzf2, Tafld, Chracl, Dnajb6, Aplp2, Batf, Bhlhe40, Fosb, Hist1h1c, Hopx, Ifih1, Ikzf3, Lass4, Lin54, Mxd1, Mxi1, Prdm1, Prf1, Rora, Rpa2, Sap30, Stat2, Stat3, Taf9b, Tbx2l, Trpsl, Xbpl, Zeb2, Atf3, Cenpcl, Lass6, Rbl, Zbtb4l, Crem, Fos12, Gtf2b, Irf7, Maff, Nr4a1, Nr4a2, Nr4a3, Obfc2a, Rb12, Rel, Rybp, Sra1, Tgif1, Tnfaip3, Uhrf2, Zbtbl, Ccdcl24, Csda, E2f3, Epas1, Hif0, H2afz, Hif1a, Ikzf5, Irf4, Nsbp1, Pim1, Rfc2, Swap70, Tfblm, 2610036L11Rik, 5133400GO4Rik, Apitdl, Blm, Brcal, Bripl, Cld, C79407, Cenpa, Cfll, Clspn, Ddxl, Dsccl, E2f7, E2f8, Ercc61, Ezh2, Fenl, Foxml, Genl, Gsg2, H2afx, Hdacl, Hdgf, Hells, Histlhle, Hist3h2a, Hjurp, Hmgb2, Hmgb3, Irfl, Irf8, Kif22, Kif4, Ligl, Lmo2, Lnp, Mbd4, Mcm2, Mcm3, Mcm4, Mcm5, Mcm6, Mcm7, Myb12, Nei13, Nusapl, Orc61, Polal, Pola2, Pole, Pole2, Polh, Polr2f, Polr2j, Ppplr8, Prim2, Psmc3ip, Rad51, Rad51c, Rad541, Rfc3, Rfc4, Rnpsl, Rpal, Smarccl, Spic, Ssrpl, Taf9, Tfdpl, Tmpo, Topbp1, Trdmt1, Uhrf1, Wdhd1, Whsc1, Zbp1, Zbtb32, Zfp367, Carl, Polg2, Atr, Lefl, Myc, Nucb2, Satb1, Tafla, Ift57, Apexl, Chd7, Chtf8, Ctnnbl, Etv3, Irf9, Myb, Mybbpla, Pms2, Preb, Sp110, Stat1, Trp53, Zfp414, App, Cdk9, Ddbl, Hsf2, Lbr, Pa2g4, Rbmsl, Rfcl, Rfc5, Tada21, Tex261, Xrcc6, and the like.

In some cases, a transcription factor may be an artificial transcription factor (ATF) including but not limited to e.g., Zinc-finger-based artificial transcription factors (including e.g., those described in Sera T. Adv Drug Deliv Rev. 2009 61(7-8):513-26; Collins et al. Curr Opin Biotechnol. 2003 14(4):371-8; Onori et al. BMC Mol Biol. 2013 14:3 the disclosures of which are incorporated herein by reference in their entirety).

In some embodiments, the intracellular domain is a transcriptional activator. In some cases, the intracellular domain comprises an amino acid sequence having at least 75%, at least 80%, at least 85%, at least 90%, at least 95%, at least 98%, at least 99%, or 100%, amino acid sequence identity to the following tetracycline-controlled transcriptional activator (tTA) amino acid sequence:

MSRLDKSKVINSALELLNEVGIEGLTTRKLAQKLGVEQPTLYWHVKNKRALLDALAIEMLDRH HTHFCPLEGESWQDFLRNNAKSFRCALLSHRDGAKVHLGTRPTEKQYETLENQLAFLCQQGFS LENALYALSAVGHFTLGCVLEDQEHQVAKEERETPTTDSMPPLLRQAIELFDHQGAEPAFLFGL ELIICGLEKQLKCESGGPADALDDFDLDMLPADALDDFDLDMLPADALDDFDLDMLPG (SEQ ID NO:33); and has a length of from about 245 amino acids to 252 amino acids (e.g., 248, 249, 250, 251, or 252 amino acids).

In some embodiments, the intracellular domain is a transcriptional activator. In some cases, the transcriptional activator is GAL4-VP16. In some cases, the transcriptional activator is GAL4-VP64. In some cases, the transcriptional activator is Tbx21. In some cases, the transcriptional activator is an engineered protein, such as a zinc finger or TALE based DNA binding domain fused to an effector domain such as VP64 (transcriptional activation) or KRAB (transcriptional repression). A variety of other transcriptional transactivators are known in the art is suitable for use.

In some cases, the intracellular domain comprises an amino acid sequence having at least 75%, at least 80%, at least 85%, at least 90%, at least 95%, at least 98%, at least 99%, or 100%, amino acid sequence identity to the following GAL4-VP64 sequence:

MKLLSSIEQACDICRLKKLKCSKEKPKCAKCLKNNWECRYSPKTKRSPLTRAHLTEVESRLERL EQLFLLIFPREDLDMILKMDSLQDIKALLTGLFVQDNVNKDAVTDRLASVETDMPLTLRQHRIS ATSSSEESSNKGQRQLTVSAAAGGSGGSGGSDALDDFDLDMLGSDALDDFDLDMLGSDALDDF DLDMLGSDALDDFDLDMLGS (SEQ ID NO:34); and has a length of from 208 to 214 amino acids (e.g., 208, 209, 210, 211, 212, 213, or 214 amino acids).

### Exemplary Embodiments

The following are non-limiting examples of suitable chimeric Notch receptor polypeptides.

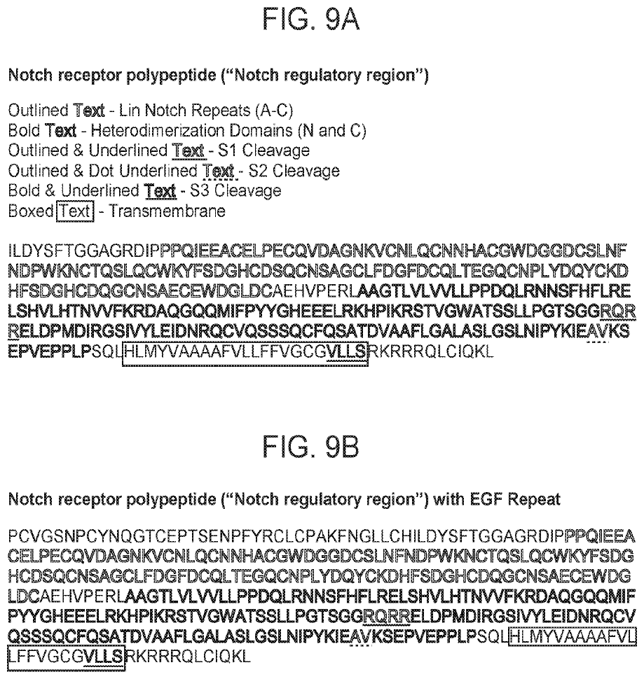

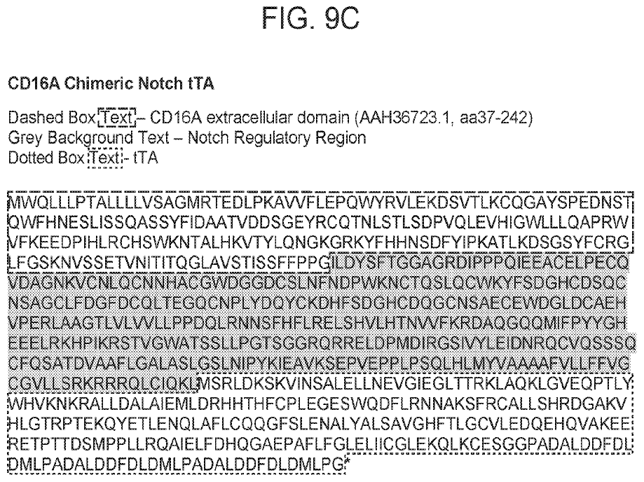

In one non-limiting embodiment, a chimeric Notch receptor polypeptide comprises a Notch receptor polypeptide that comprises, in order from N-terminus to C-terminus: i) Lin Notch Repeats A-C (an LNR segment); ii) a heterodimerization domain (an HD-N segment and an HD-C segment); iii) a TM domain; and comprises an 51 proteolytic cleavage site, an S2 proteolytic cleavage site, and an S3 proteolytic cleavage site. An example of such a Notch receptor polypeptide is depicted in FIG. 9A. In FIG. 9A, Lin Notch Repeats A-C (an LNR segment) have the following amino acid sequence:

PPQIEEACELPECQVDAGNKVCNLQCNNHACGWDGGDCSLNFNDPWKNCTQSLQCWKYFSDG HCDSQCNSAGCLFDGFDCQLTEGQCNPLYDQYCKDHFSDGHCDQGCNSAECEWDGLDC (SEQ ID NO:15); the heterodimerization domain (an HD-N segment and an HD-C segment) has the following amino acid sequence:
AAGTLVLVVLLPPDQLRNNSFHFLRELSHVLHTNVVFKRDAQGQQMIFPYYGHEEELRKHPIKR STVGWATSSLLPGTSGG [symbol]
ELDPMDIRGSIVYLEIDNRQCVQSSSQCFQSATDVAAFLGALA SLGSLNIPYKIE [symbol]
KSEPVEPPLP (SEQ ID NO:16), where the S1 proteolytic cleavage site includes the sequence RQRR (SEQ ID NO:37), and the S2 proteolytic cleavage site includes the sequence AV; and the TM domain has the following amino acid sequence: HLMYVAAAAFVLLFFVGCGVLLS (SEQ ID NO:17), where the S3 proteolytic cleavage site includes the sequence VLLS (SEQ ID NO:39).

In one non-limiting embodiment, a chimeric Notch receptor polypeptide comprises a Notch receptor polypeptide that comprises, in order from N-terminus to C-terminus: i) an EGF repeat; ii) Lin Notch Repeats A-C (an LNR segment); iii) a heterodimerization domain (an HD-N segment and an HD-C segment); iv) a TM domain; and comprises an 51 proteolytic cleavage site, an S2 proteolytic cleavage site, and an S3 proteolytic cleavage site. An example of such a Notch receptor polypeptide is depicted in FIG. 9B. In FIG. 9B, the EGF repeat has the following amino acid sequence:

PCVGSNPCYNQGTCEPTSENPFYRCLCPAKFNGLLCH (SEQ ID NO:20); and the LNR segment, the heterodimerization domain, the TM domain, the S1 proteolytic cleavage site, the S2 proteolytic cleavage site, and the S3 proteolytic cleavage site are as depicted in FIG. 9A.

In one non-limiting embodiment, a chimeric Notch receptor polypeptide comprises a Notch receptor polypeptide that comprises the following amino acid sequence:

(SEQ ID NO: 13)
IPYKIEAVKSEPVEPPLPSQLHLMYVAAAAFVLLFFVGCGVLLSRKRRR
QLCIQKL;

where the TM domain is underlined; where the Notch receptor polypeptide comprises an S2 proteolytic cleavage site and an S3 proteolytic cleavage site; where the Notch receptor polypeptide has a length of 56 amino acids.

In one non-limiting embodiment, a chimeric Notch receptor polypeptide comprises, in order from N-terminus to C-terminus: a) an Fc receptor FcyIIIa (CD16A); b) a Notch receptor polypeptide comprising: i) an LNR segment; ii) a heterodimerization domain (an HD-N segment and an HD-C segment); and iii) a TM domain, where the Notch receptor polypeptide comprises one or more ligand-inducible proteolytic cleavage sites; and c) an intracellular domain, where the intracellular domain is a transcriptional activator. In one non-limiting embodiment, a chimeric Notch receptor polypeptide of the present disclosure comprises, in order from N-terminus to C-terminus: a) an Fc receptor FcyIIIa (CD16A); a Notch receptor polypeptide comprising: i) an LNR segment; ii) a heterodimerization domain (an HD-N segment and an HD-C segment); and iii) a TM domain, where the Notch receptor polypeptide comprises one or more ligand-inducible proteolytic cleavage sites; and c) an intracellular domain, where the intracellular domain is a tTA transcription factor. An example of such a chimeric Notch receptor polypeptide is depicted in FIG. 9C. The locations of S1, S2, and S3 cleavage sites are depicted in FIG. 9A. In FIG. 9C, the CD16A has the following amino acid sequence:

MWQLLLPTALLLLVSAGMRTEDLPKAVVFLEPQWYRVLEKDSVTLKCQGAYSPEDNSTQWFH NESLISSQASSYFIDAATVDDSGEYRCQTNLSTLSDPVQLEVHIGWLLLQAPRWVFKEEDPIHLR CHSWKNTALHKVTYLQNGKGRKYFHHNSDFYIPKATLKDSGSYFCRGLFGSKNVSSETVNITIT QGLAVSTISSFFPPG (SEQ ID NO:42); the Notch receptor polypeptide has the amino acid sequence depicted in FIG. 9A; and the tTA transcription factor has the following amino acid sequence:

(SEQ ID NO: 33)
MSRLDKSKVINSALELLNEVGIEGLTTRKLAQKLGVEQPTLYWHVKNKR
ALLDALAIEMLDRHHTHFCPLEGESWQDFLRNNAKSFRCALLSHRDGAK
VHLGTRPTEKQYETLENQLAFLCQQGFSLENALYALSAVGHFTLGCVLE
DQEHQVAKEERETPTTDSMPPLLRQAIELFDHQGAEPAFLFGLELIICG
LEKQLKCESGGPADALDDFDLDMLPADALDDFDLDMLPADALDDFDLDM
LPG.

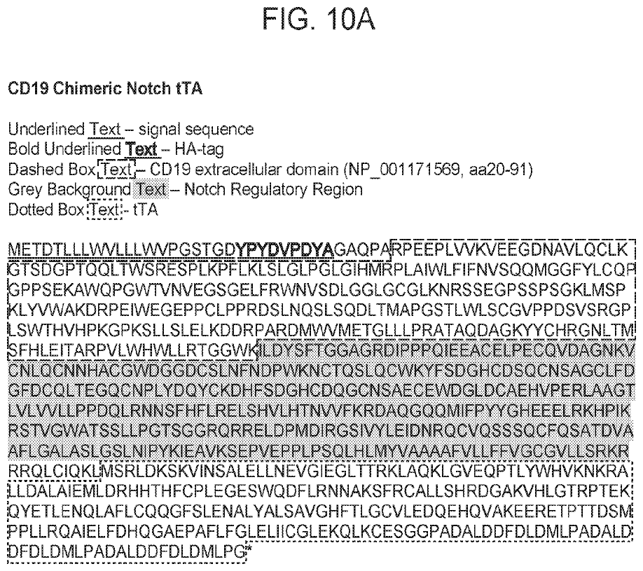

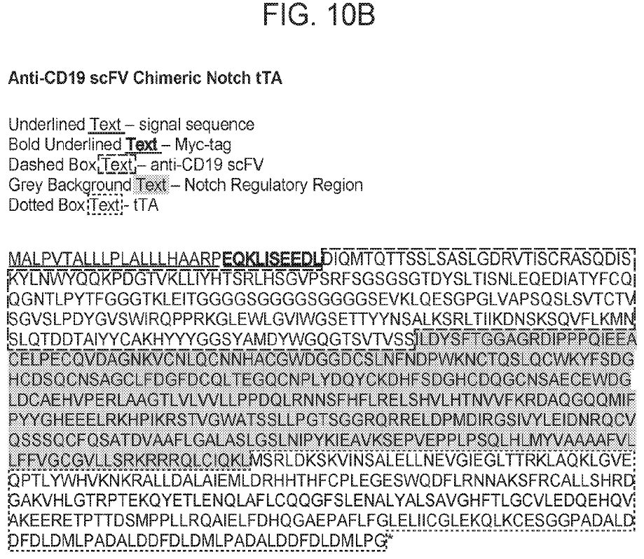

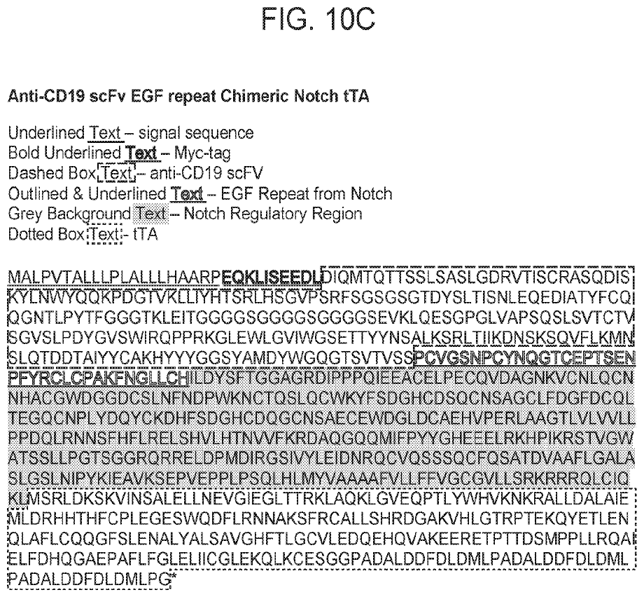

In one non-limiting embodiment, a chimeric Notch receptor polypeptide comprises, in order from N-terminus to C-terminus: a) a cell surface antigen; b) a Notch receptor polypeptide comprising: i) an LNR segment; ii) a heterodimerization domain (an HD-N segment and an HD-C segment); and iii) a TM domain, where the Notch receptor polypeptide comprises one or more ligand-inducible proteolytic cleavage sites; and c) an intracellular domain, where the intracellular domain is a transcriptional activator. In one non-limiting embodiment, a chimeric Notch receptor polypeptide comprises, in order from N-terminus to C-terminus: a) a CD19 polypeptide; b) a Notch receptor polypeptide comprising: i) an LNR segment; ii) a heterodimerization domain (an HD-N segment and an HD-C segment); and iii) a TM domain where the Notch receptor polypeptide comprises one or more ligand-inducible proteolytic cleavage sites; and c) an intracellular domain, where the intracellular domain is a tTA transcription factor. An example of such a chimeric Notch receptor polypeptide is depicted in FIG. 10A. In FIG. 10A, the CD19 polypeptide has the following amino acid sequence:

RPEEPLVVKVEEGDNAVLQCLKGTSDGPTQQLTWSRESPLKPFLKLSLGLPGLGIHMRPLAIWLF IFNVS QQMGGFYLCQPGPPSEKAWQPGWTVNVEGSGELFRWNVSDLGGLGCGLKNRSSEGPSS PSGKLMSPKLYVWAKDRPEIWEGEPPCLPPRDSLNQSLSQDLTMAPGSTLWLSCGVPPDSVSRG PLSWTHVHPKGPKSLLSLELKDDRPARDMWVMETGLLLPRATAQDAGKYYCHRGNLTMSFHL EITARPVLWHWLLRTGGWK (SEQ ID NO:44); the Notch receptor polypeptide includes the amino acid sequence depicted in FIG. 9A; and the tTA transcription factor has the amino acid sequence depicted in FIG. 9C.

In one non-limiting embodiment, a chimeric Notch receptor polypeptide comprises, in order from N-terminus to C-terminus: a) an antibody; b) a Notch receptor polypeptide comprising: i) an LNR segment; ii) a heterodimerization domain (an HD-N segment and an HD-C segment); and iii) a TM domain, where the Notch receptor polypeptide comprises one or more ligand-inducible proteolytic cleavage sites; and c) an intracellular domain, where the intracellular domain is a transcriptional activator. In one non-limiting embodiment, a chimeric Notch receptor polypeptide comprises, in order from N-terminus to C-terminus: a) an antibody specific for a cell surface antigen; b) a Notch receptor polypeptide comprising: i) an LNR segment; ii) a heterodimerization domain (an HD-N segment and an HD-C segment); and iii) a TM domain, where the Notch receptor polypeptide comprises one or more ligand-inducible proteolytic cleavage sites; and c) an intracellular domain, where the intracellular domain is a transcriptional activator. In one non-limiting embodiment, a chimeric Notch receptor polypeptide of the present disclosure comprises, in order from N-terminus to C-terminus: a) an anti-CD19 scFv; b) a Notch receptor polypeptide comprising: i) an LNR segment; ii) a heterodimerization domain (an HD-N segment and an HD-C segment); and iii) a TM domain where the Notch receptor polypeptide comprises one or more ligand-inducible proteolytic cleavage sites; and c) an intracellular domain, where the intracellular domain is a tTA transcription factor. An example of such a chimeric Notch receptor polypeptide is depicted in FIG. 10B. In FIG. 10B, the anti-CD19 scFv has the following amino acid sequence:

DIQMTQTTSSLSASLGDRVTISCRASQDISKYLNWYQQKPDGTVKLLIYHTSRLHSGVPSRFSGS GSGTDYSLTISNLEQEDIATYFCQQGNTLPYTFGGGTKLEITGGGGSGGGGSGGGGSEVKLQESG PGLVAPSQSLSVTCTVSGVSLPDYGVSWIRQPPRKGLEWLGVIWGSETTYYNSALKSRLTIIKDN SKSQVFLKMNSLQTDDTAIYYCAKHYYYGGSYAMDYWGQGTSVTVSS (SEQ ID NO:45); the Notch receptor polypeptide includes the amino acid sequence depicted in FIG. 9A; and the tTA transcription factor has the amino acid sequence depicted in FIG. 9C.

In one non-limiting embodiment, a chimeric Notch receptor polypeptide of the present disclosure comprises, in order from N-terminus to C-terminus: a) an antibody; b) a Notch receptor polypeptide comprising: i) an EGF repeat; ii) an LNR segment; iii) a heterodimerization domain (an HD-N segment and an HD-C segment); and iv) a TM domain, where the Notch receptor polypeptide comprises one or more ligand-inducible proteolytic cleavage sites; and c) an intracellular domain, where the intracellular domain is a transcriptional activator. In one non-limiting embodiment, a chimeric Notch receptor polypeptide of the present disclosure comprises, in order from N-terminus to C-terminus: a) an antibody specific for a cell surface antigen; b) a Notch receptor polypeptide comprising: i) an EGF repeat; ii) an LNR segment; iii) a heterodimerization domain (an HD-N segment and an HD-C segment); and iv) a TM domain, where the Notch receptor polypeptide comprises one or more ligand-inducible proteolytic cleavage sites; and c) an intracellular domain, where the intracellular domain is a transcriptional activator. In one non-limiting embodiment, a chimeric Notch receptor polypeptide comprises, in order from N-terminus to C-terminus: a) an anti-CD19 scFv; b) a Notch receptor polypeptide comprising: i) an EGF repeat; ii) an LNR segment; iii) a heterodimerization domain (an HD-N segment and an HD-C segment); and iv) a TM domain, where the Notch receptor polypeptide comprises one or more ligand-inducible proteolytic cleavage sites; and c) an intracellular domain, where the intracellular domain is a tTA transcription factor. An example of such a chimeric Notch receptor polypeptide is depicted in FIG. 10C. In FIG. 10C, the anti-CD19 scFv has the following amino acid sequence:

DIQMTQTTSSLSASLGDRVTISCRASQDISKYLNWYQQKPDGTVKLLIYHTSRLHSGVPSRFSGS GSGTDYSLTISNLEQEDIATYFCQQGNTLPYTFGGGTKLEITGGGGSGGGGSGGGGSEVKLQESG PGLVAPSQSLSVTCTVSGVSLPDYGVSWIRQPPRKGLEWLGVIWGSETTYYNSALKSRLTIIKDN SKSQVFLKMNSLQTDDTAIYYCAKHYYYGGSYAMDYWGQGTSVTVSS (SEQ ID NO:45); the Notch receptor polypeptide includes the amino acid sequence depicted in FIG. 9B; and the tTA transcription factor has the amino acid sequence depicted in FIG. 9C.

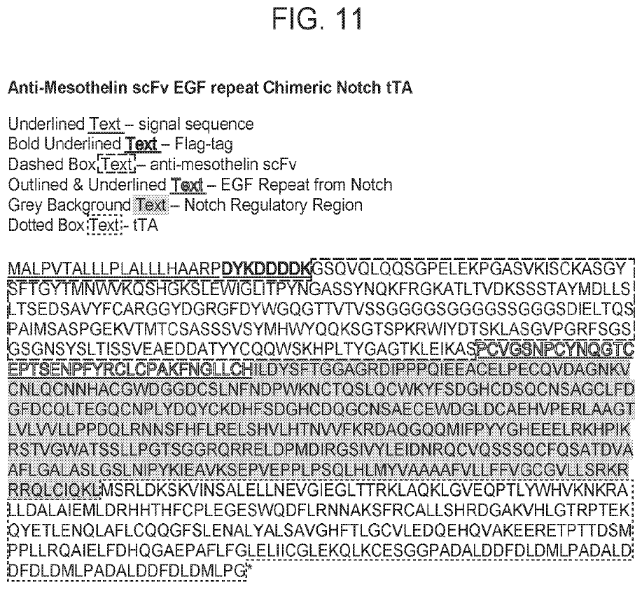

In one non-limiting embodiment, a chimeric Notch receptor polypeptide comprises, in order from N-terminus to C-terminus: a) an antibody; b) a Notch receptor polypeptide comprising: i) an EGF repeat; ii) an LNR segment; iii) a heterodimerization domain (an HD-N segment and an HD-C segment); and iv) a TM domain, where the Notch receptor polypeptide comprises one or more ligand-inducible proteolytic cleavage sites; and c) an intracellular domain, where the intracellular domain is a transcriptional activator. In one non-limiting embodiment, a chimeric Notch receptor polypeptide comprises, in order from N-terminus to C-terminus: a) an antibody specific for a cell surface antigen, e.g., a cell surface antigen present on the surface of a cancer cell (e.g., a cancer-specific antigen); b) a Notch receptor polypeptide comprising: i) an EGF repeat; ii) an LNR segment; iii) a heterodimerization domain (an HD-N segment and an HD-C segment); and iv) a TM domain, where the Notch receptor polypeptide comprises one or more ligand-inducible proteolytic cleavage sites; and c) an intracellular domain, where the intracellular domain is a transcriptional activator. In one non-limiting embodiment, a chimeric Notch receptor polypeptide of the present disclosure comprises, in order from N-terminus to C-terminus: a) an anti-mesothelin scFv; b) a Notch receptor polypeptide comprising: i) an EGF repeat; ii) an LNR segment; iii) a heterodimerization domain (an HD-N segment and an HD-C segment); and iv) a TM domain, where the Notch receptor polypeptide comprises one or more ligand-inducible proteolytic cleavage sites; and c) an intracellular domain, where the intracellular domain is a tTA transcription factor. An example of such a chimeric Notch receptor polypeptide is depicted in FIG. 11. In FIG. 11, the anti-mesothelin scFv has the following amino acid sequence:

GSQVQLQQSGPELEKPGASVKISCKASGYSFTGYTMNWVKQSHGKSLEWIGLITPYNGASSYNQ KFRGKATLTVDKSSSTAYMDLLSLTSEDSAVYFCARGGYDGRGFDYWGQGTTVTVSSGGGGS GGGGSSGGGSDIELTQSPAIMSASPGEKVTMTCSASSSVSYMHWYQQKSGTSPKRWIYDTSKLA SGVPGRFSGSGSGNSYSLTISSVEAEDDATYYCQQWSKHPLTYGAGTKLEIKAS (SEQ ID NO:47); the Notch receptor polypeptide includes the amino acid sequence depicted in FIG. 9B; and the tTA transcription factor has the amino acid sequence depicted in FIG. 9C.

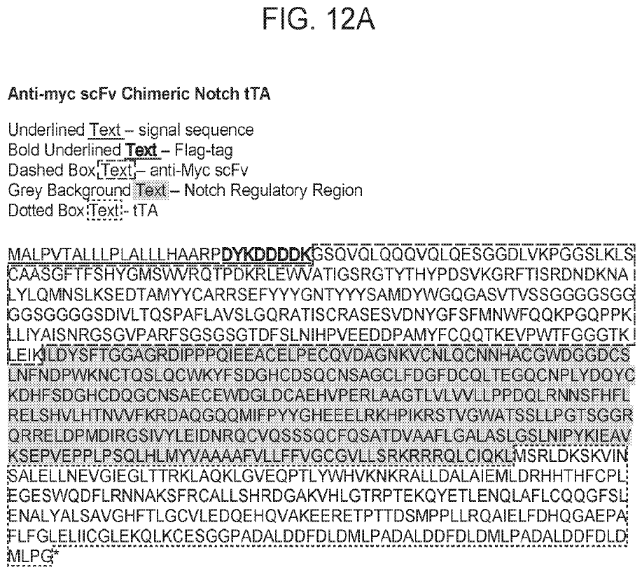

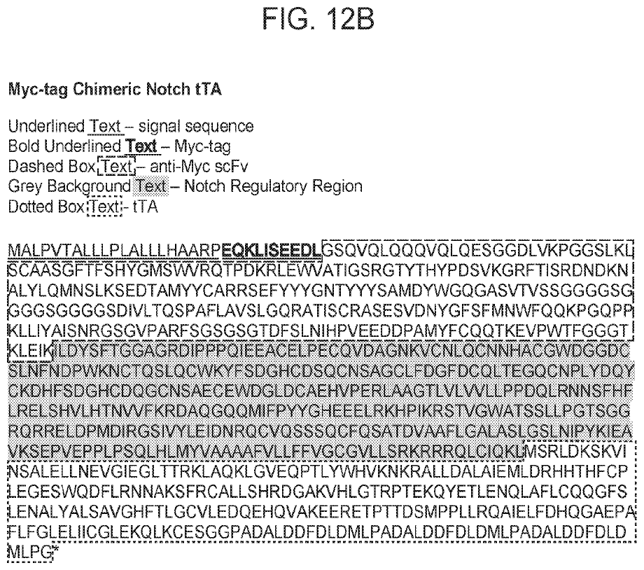

In one non-limiting embodiment, a chimeric Notch receptor polypeptide comprises, in order from N-terminus to C-terminus: a) an antibody; b) a Notch receptor polypeptide comprising: i) an LNR segment; ii) a heterodimerization domain (an HD-N segment and an HD-C segment); and iii) a TM domain, where the Notch receptor polypeptide comprises one or more ligand-inducible proteolytic cleavage sites; and c) an intracellular domain, where the intracellular domain is a transcriptional activator. In one non-limiting embodiment, a chimeric Notch receptor polypeptide comprises, in order from N-terminus to C-terminus: a) an antibody specific for a transcription factor; b) a Notch receptor polypeptide comprising: i) an LNR segment; ii) a heterodimerization domain (an HD-N segment and an HD-C segment); and iii) a TM domain, where the Notch receptor polypeptide comprises one or more ligand-inducible proteolytic cleavage sites; and c) an intracellular domain, where the intracellular domain is a transcriptional activator. In one non-limiting embodiment, a chimeric Notch receptor polypeptide comprises, in order from N-terminus to C-terminus: a) an anti-myc scFv; b) a Notch receptor polypeptide comprising: i) an LNR segment; ii) a heterodimerization domain (an HD-N segment and an HD-C segment); and iii) a TM domain, where the Notch receptor polypeptide comprises one or more ligand-inducible proteolytic cleavage sites; and c) an intracellular domain, where the intracellular domain is a tTA transcription factor. Examples of such a chimeric Notch receptor polypeptide are depicted in FIG. 12A and 12B. In FIG. 12A and FIG. 12B, the anti-Myc scFv has the following amino acid sequence:

GSQVQLQQQVQLQESGGDLVKPGGSLKLSCAASGFTFSHYGMSWVRQTPDKRLEWVATIGSR GTYTHYPDSVKGRFTISRDNDKNALYLQMNSLKSEDTAMYYCARRSEFYYYGNTYYYSAMDY WGQGASVTVSSGGGGSGGGGSGGGGSDIVLTQSPAFLAVSLGQRATISCRASESVDNYGFSFM NWFQQKPGQPPKLLIYAISNRGSGVPARFSGSGSGTDFSLNIHPVEEDDPAMYFCQQTKEVPWT FGGGTKLEIK (SEQ ID NO:48); the Notch receptor polypeptide includes the amino acid sequence depicted in FIG. 9A; and the tTA transcription factor has the amino acid sequence depicted in FIG. 9C.

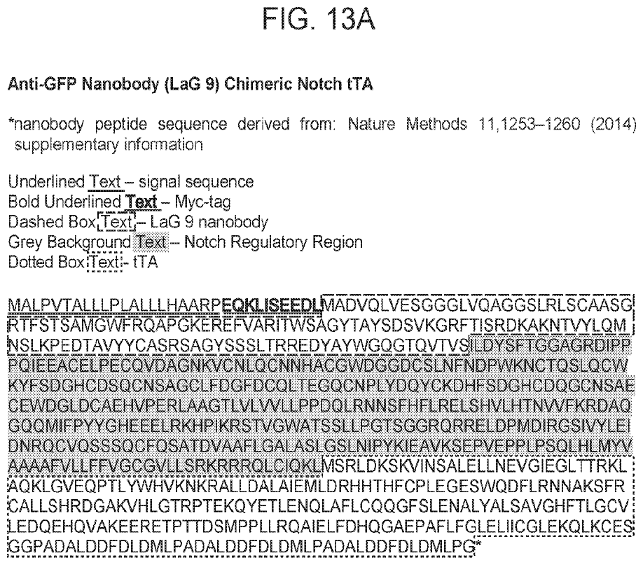

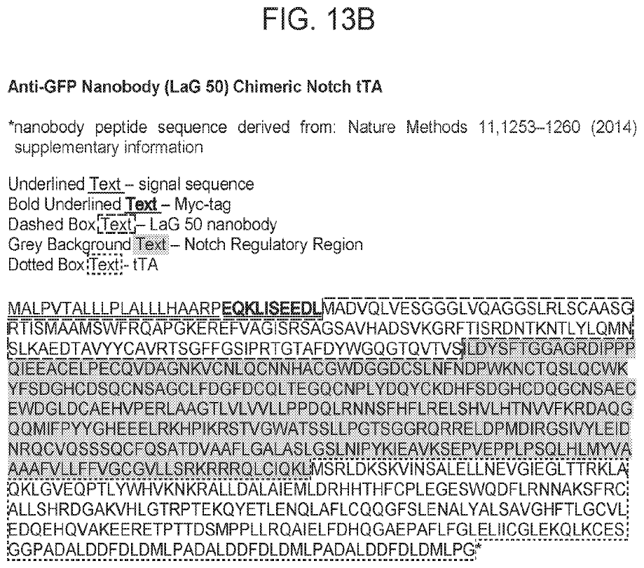

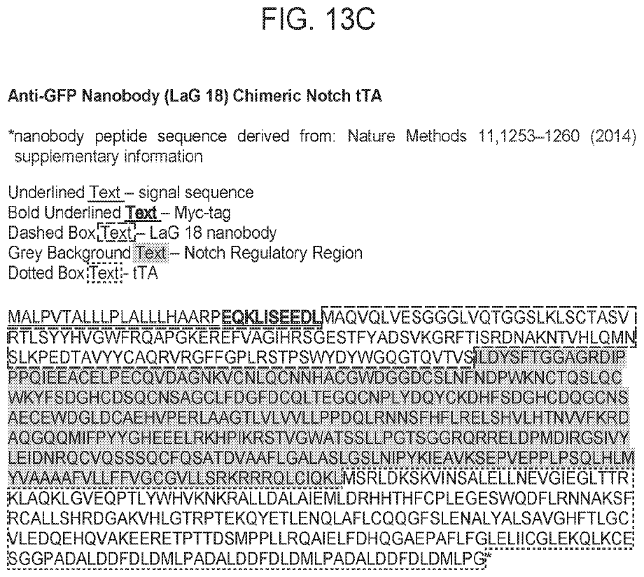

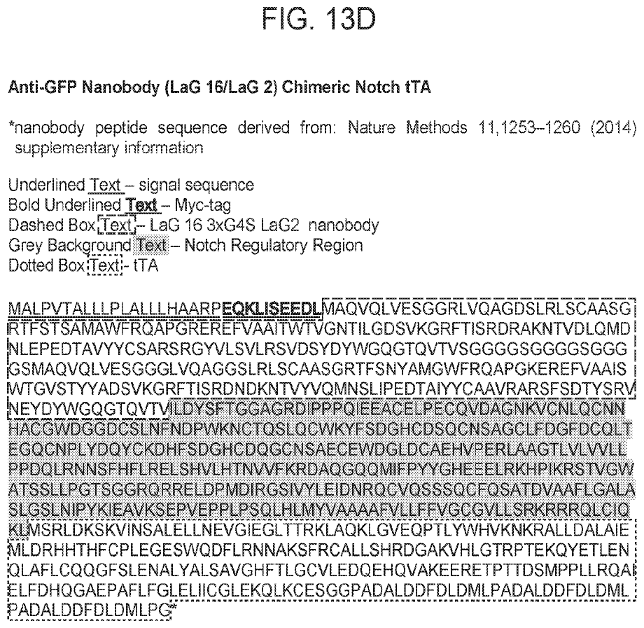

In one non-limiting embodiment, a chimeric Notch receptor polypeptide of the present disclosure comprises, in order from N-terminus to C-terminus: a) a nanobody; b) a Notch receptor polypeptide comprising: i) an LNR segment; ii) a heterodimerization domain (an HD-N segment and an HD-C segment); and iii) a TM domain, where the Notch receptor polypeptide comprises one or more ligand-inducible proteolytic cleavage sites; and c) an intracellular domain, where the intracellular domain is a transcriptional activator. In one non-limiting embodiment, a chimeric Notch receptor polypeptide of the present disclosure comprises, in order from N-terminus to C-terminus: a) a LaG 9 nanobody; b) a Notch receptor polypeptide comprising: i) an LNR segment; ii) a heterodimerization domain (an HD-N segment and an HD-C segment); and iii) a TM domain, where the Notch receptor polypeptide comprises one or more ligand-inducible proteolytic cleavage sites; and c) an intracellular domain, where the intracellular domain is a tTA transcription factor. An example of such a chimeric Notch receptor polypeptide is depicted in FIG. 13A. In FIG. 13A, the LaG 9 nanobody has the following amino acid sequence:

MADVQLVESGGGLVQAGGSLRLSCAASGRTFSTSAMGWFRQAPGKEREFVARITWSAGYTAY SDSVKGRFTISRDKAKNTVYLQMNSLKPEDTAVYYCASRSAGYSSSLTRREDYAYWGQGTQVT VS (SEQ ID NO:49); the Notch receptor polypeptide includes the amino acid sequence depicted in FIG. 9A; and the tTA transcription factor has the amino acid sequence depicted in FIG. 9C.

In one non-limiting embodiment, a chimeric Notch receptor polypeptide comprises, in order from N-terminus to C-terminus: a) a nanobody; b) a Notch receptor polypeptide comprising: i) an LNR segment; ii) a heterodimerization domain (an HD-N segment and an HD-C segment); and iii) a TM domain, where the Notch receptor polypeptide comprises one or more ligand-inducible proteolytic cleavage sites; and c) an intracellular domain, where the intracellular domain is a transcriptional activator. In one non-limiting embodiment, a chimeric Notch receptor polypeptide comprises, in order from N-terminus to C-terminus: a) a LaG 50 nanobody; b) a Notch receptor polypeptide comprising: i) an LNR segment; ii) a heterodimerization domain (an HD-N segment and an HD-C segment); and iii) a TM domain, where the Notch receptor polypeptide comprises one or more ligand-inducible proteolytic cleavage sites; and c) an intracellular domain, where the intracellular domain is a tTA transcription factor. An example of such a chimeric Notch receptor polypeptide is depicted in FIG. 13B. In FIG. 13B, the LaG 50 nanobody has the following amino acid sequence:

MADVQLVESGGGLVQAGGSLRLSCAASGRTISMAAMSWFRQAPGKEREFVAGISRSAGSAVH ADSVKGRFTISRDNTKNTLYLQMNSLKAEDTAVYYCAVRTSGFFGSIPRTGTAFDYWGQGTQV TVS (SEQ ID NO:50); the Notch receptor polypeptide includes the amino acid sequence depicted in FIG. 9A; and the tTA transcription factor has the amino acid sequence depicted in FIG. 9C.

In one non-limiting embodiment, a chimeric Notch receptor polypeptide comprises, in order from N-terminus to C-terminus: a) a nanobody; b) a Notch receptor polypeptide comprising: i) an LNR segment; ii) a heterodimerization domain (an HD-N segment and an HD-C segment); and iii) a TM domain, where the Notch receptor polypeptide comprises one or more ligand-inducible proteolytic cleavage sites; and c) an intracellular domain, where the intracellular domain is a transcriptional activator. In one non-limiting embodiment, a chimeric Notch receptor polypeptide comprises, in order from N-terminus to C-terminus: a) a LaG 18 nanobody; b) a Notch receptor polypeptide comprising: i) an LNR segment; ii) a heterodimerization domain (an HD-N segment and an HD-C segment); and iii) a TM domain, where the Notch receptor polypeptide comprises one or more ligand-inducible proteolytic cleavage sites; and c) an intracellular domain, where the intracellular domain is a tTA transcription factor. An example of such a chimeric Notch receptor polypeptide is depicted in FIG. 13C. In FIG. 13C, the LaG 18 nanobody has the following amino acid sequence:

MAQVQLVESGGGLVQTGGSLKLSCTASVRTLSYYHVGWFRQAPGKEREFVAGIHRSGESTFYA DSVKGRFTISRDNAKNTVHLQMNSLKPEDTAVYYCAQRVRGFFGPLRSTPSWYDYWGQGTQV TVS (SEQ ID NO:51); the Notch receptor polypeptide includes the amino acid sequence depicted in FIG. 9A; and the tTA transcription factor has the amino acid sequence depicted in FIG. 9C.

In one non-limiting embodiment, a chimeric Notch receptor polypeptide comprises, in order from N-terminus to C-terminus: a) a nanobody; b) a Notch receptor polypeptide comprising: i) an LNR segment; ii) a heterodimerization domain (an HD-N segment and an HD-C segment); and iii) a TM domain, where the Notch receptor polypeptide comprises one or more ligand-inducible proteolytic cleavage sites; and c) an intracellular domain, where the intracellular domain is a transcriptional activator. In one non-limiting embodiment, a chimeric Notch receptor polypeptide comprises, in order from N-terminus to C-terminus: a) a LaG 16/LaG 2 nanobody; b) a Notch receptor polypeptide comprising: i) an LNR segment; ii) a heterodimerization domain (an HD-N segment and an HD-C segment); and iii) a TM domain, where the Notch receptor polypeptide comprises one or more ligand-inducible proteolytic cleavage sites; and c) an intracellular domain, where the intracellular domain is a tTA transcription factor. An example of such a chimeric Notch receptor polypeptide is depicted in FIG. 13D. In FIG. 13D, the LaG 16/LaG 2 nanobody has the following amino acid sequence:

MAQVQLVESGGRLVQAGDSLRLSCAASGRTFSTSAMAWFRQAPGREREFVAAITWTVGNTILG DSVKGRFTISRDRAKNTVDLQMDNLEPEDTAVYYCSARSRGYVLSVLRSVDSYDYWGQGTQV TVSGGGGSGGGGSGGGGSMAQVQLVESGGGLVQAGGSLRLSCAASGRTFSNYAMGWFRQAP GKEREFVAAISWTGVSTYYADSVKGRFTISRDNDKNTVYVQMNSLIPEDTAIYYCAAVRARSFS DTYSRVNEYDYWGQGTQVTV (SEQ ID NO:52); the Notch receptor polypeptide includes the amino acid sequence depicted in FIG. 9A; and the tTA transcription factor has the amino acid sequence depicted in FIG. 9C.

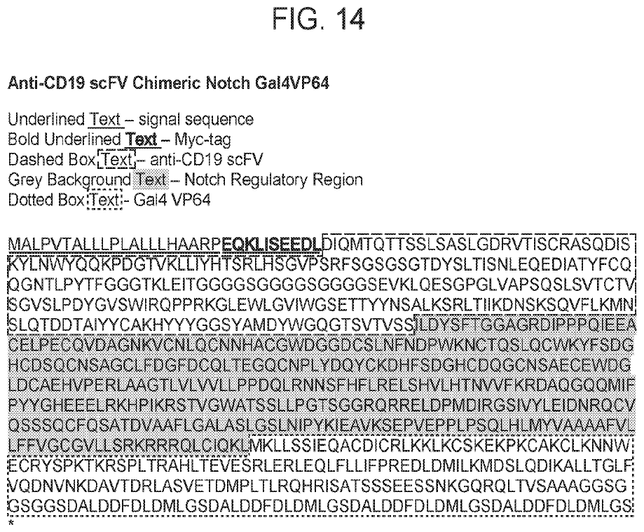

In one non-limiting embodiment, a chimeric Notch receptor polypeptide comprises, in order from N-terminus to C-terminus: a) an antibody; b) a Notch receptor polypeptide comprising: i) an LNR segment; ii) a heterodimerization domain (an HD-N segment and an HD-C segment); and iii) a TM domain, where the Notch receptor polypeptide comprises one or more ligand-inducible proteolytic cleavage sites; and c) an intracellular domain, where the intracellular domain is a transcriptional activator. In one non-limiting embodiment, a chimeric Notch receptor polypeptide comprises, in order from N-terminus to C-terminus: a) an antibody specific for a cell surface antigen; b) a Notch receptor polypeptide comprising: i) an LNR segment; ii) a heterodimerization domain (an HD-N segment and an HD-C segment); and iii) a TM domain, where the Notch receptor polypeptide comprises one or more ligand-inducible proteolytic cleavage sites; and c) an intracellular domain, where the intracellular domain is a transcriptional activator. In one non-limiting embodiment, a chimeric Notch receptor polypeptide comprises, in order from N-terminus to C-terminus: a) an anti-CD19 scFv; b) a Notch receptor polypeptide comprising: i) an LNR segment; ii) a heterodimerization domain (an HD-N segment and an HD-C segment); and iii) a TM domain where the Notch receptor polypeptide comprises one or more ligand-inducible proteolytic cleavage sites; and c) an intracellular domain, where the intracellular domain is Ga14-VP64 transcriptional activator. An example of such a chimeric Notch receptor polypeptide is depicted in FIG. 14. In FIG. 14, the anti-CD19 scFv has the following amino acid sequence:

DIQMTQTTSSLSASLGDRVTISCRASQDISKYLNWYQQKPDGTVKLLIYHTSRLHSGVPSRFSGS GSGTDYSLTISNLEQEDIATYFCQQGNTLPYTFGGGTKLEITGGGGSGGGGSGGGGSEVKLQESG PGLVAPSQSLSVTCTVSGVSLPDYGVSWIRQPPRKGLEWLGVIWGSETTYYNSALKSRLTIIKDN SKSQVFLKMNSLQTDDTAIYYCAKHYYYGGSYAMDYWGQGTSVTVSS (SEQ ID NO:45); the Notch receptor polypeptide includes the amino acid sequence depicted in FIG. 9A; and the Ga14-VP64 transcriptional activator has the following amino acid sequence:

(SEQ ID NO: 34)
MKLLSSIEQACDICRLKKLKCSKEKPKCAKCLKNNWECRYSPKTKRSPL
TRAHLTEVESRLERLEQLFLLIFPREDLDMILKMDSLQDIKALLTGLFV
QDNVNKDAVTDRLASVETDMPLTLRQHRISATSSSEESSNKGQRQLTVS
AAAGGSGGSGGSDALDDFDLDMLGSDALDDFDLDMLGSDALDDFDLDML
GSDALDDFDLDMLGS.

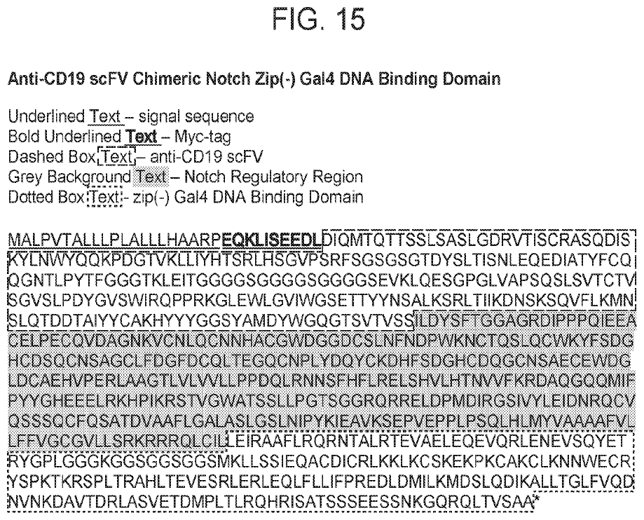

In one non-limiting embodiment, a chimeric Notch receptor polypeptide comprises, in order from N-terminus to C-terminus: a) an antibody; b) a Notch receptor polypeptide comprising: i) an LNR segment; ii) a heterodimerization domain (an HD-N segment and an HD-C segment); and iii) a TM domain, where the Notch receptor polypeptide comprises one or more ligand-inducible proteolytic cleavage sites; and c) an intracellular domain, where the intracellular domain is a DNA binding polypeptide. In one non-limiting embodiment, a chimeric Notch receptor polypeptide comprises, in order from N-terminus to C-terminus: a) an anti-CD19 scFv; b) a Notch receptor polypeptide comprising: i) an LNR segment; ii) a heterodimerization domain (an HD-N segment and an HD-C segment); and iii) a TM domain where the Notch receptor polypeptide comprises one or more ligand-inducible proteolytic cleavage sites; and c) an intracellular domain, where the intracellular domain is a Zip(−) Gal4 DNA binding polypeptide. An example of such a chimeric Notch receptor polypeptide is depicted in FIG. 15. In FIG. 15, the anti-CD19 scFv has the amino acid sequence depicted in FIG. 14; the Notch receptor polypeptide includes the amino acid sequence depicted in FIG. 9A; and the Zip(−) Gal4 DNA binding polypeptide has the following amino acid sequence:

(SEQ ID NO: 55)
LEIRAAFLRQRNTALRTEVAELEQEVQRLENEVSQYETRYGPLGGGKGG
SGGSGGSMKLLSSIEQACDICRLKKLKCSKEKPKCAKCLKNNWECRYSP
KTKRSPLTRAHLTEVESRLERLEQLFLLIFPREDLDMILKMDSLQDIKA
LLTGLFVQDNVNKDAVTDRLASVETDMPLTLRQHRISATSSSEESSNKG
QRQLTVSAA.

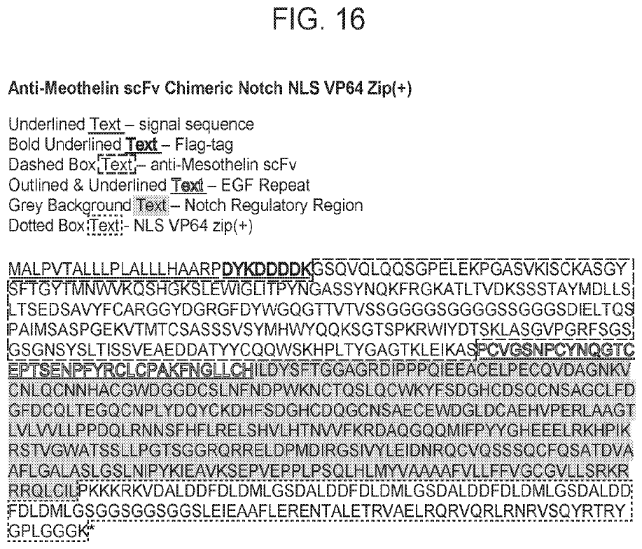

In one non-limiting embodiment, a chimeric Notch receptor polypeptide comprises, in order from N-terminus to C-terminus: a) an antibody; b) a Notch receptor polypeptide comprising: i) an EGF repeat; ii) an LNR segment; iii) a heterodimerization domain (an HD-N segment and an HD-C segment); and iv) a TM domain, where the Notch receptor polypeptide comprises one or more ligand-inducible proteolytic cleavage sites; and c) an intracellular domain, where the intracellular domain is a transcriptional activator. In one non-limiting embodiment, a chimeric Notch receptor polypeptide comprises, in order from N-terminus to C-terminus: a) an anti-mesothelin scFv; b) a Notch receptor polypeptide comprising: i) an EGF repeat; ii) an LNR segment; iii) a heterodimerization domain (an HD-N segment and an HD-C segment); and iv) a TM domain, where the Notch receptor polypeptide comprises one or more ligand-inducible proteolytic cleavage sites; and c) an intracellular domain, where the intracellular domain is VP64 Zip(+) comprising an NLS. An example of such a chimeric Notch receptor polypeptide is depicted in FIG. 16. In FIG. 16, the anti-mesothelin scFv has the following amino acid sequence:

GSQVQLQQSGPELEKPGASVKISCKASGYSFTGYTMNWVKQSHGKSLEWIGLITPYNGASSYNQ KFRGKATLTVDKSSSTAYMDLLSLTSEDSAVYFCARGGYDGRGFDYWGQGTTVTVSSGGGGS GGGGSSGGGSDIELTQSPAIMSASPGEKVTMTCSASSSVSYMHWYQQKSGTSPKRWIYD (SEQ ID NO:56); the Notch receptor polypeptide includes the amino acid sequence depicted in FIG. 9B; and the VP64 Zip(+) transcriptional activator has the following amino acid sequence:

(SEQ ID NO: 57)
PKKKRKVDALDDFDLDMLGSDALDDFDLDMLGSDALDDFDLDMLGSDAL
DDFDLDMLGSGGSGGSGGSLEIEAAFLERENTALETRVAELRQRVQRLR
NRVSQYRTRYGPLGGGK.

### Force Sensor Cleavage Domain-Containing Chimeric Polypeptides

In some cases, the chimeric polypeptide is a force sensor cleavage domain-containing chimeric polypeptides (also referred to herein as “A2 chimeric polypeptides”). The term “force sensor cleavage domain”, as used herein, refers to a polypeptide domain of a force sensitive protein that, upon the application of force, is cleavable, e.g., by a protease, including non-Notch force sensor cleavage domains. By “non-Notch force sensor cleavage domain”, as used herein, is meant a cleavage domain of a force sensitive protein that is not, or is not derived from, a Notch protein. Such, non-Notch force sensor cleavage domains will not include a Notch negative regulatory region (NRR), Notch cleavage site(s) (e.g., S1, S2 or S3 sites) or any other portion of a Notch protein. However, in some instances, a non-Notch force sensor cleavage domain may be present in a polypeptide with other Notch-derived domains, such as domains of a Notch protein other than the Notch force sensitive cleavage domain Force sensor cleavage domains may be derived from force sensitive proteins from various species including but not limited to e.g., invertebrates (e.g., insects) and vertebrates (e.g., mammals such as mouse, rat, human and non-human primates), etc.

An A2 chimeric polypeptide comprises, from N-terminal to C-terminal and in covalent linkage: a) an extracellular domain comprising an antibody specific for a target antigen; b) a non-Notch force sensor cleavage domain comprising a proteolytic cleavage site; c) a cleavable transmembrane domain; and d) an intracellular domain comprising a Notch intracellular signaling domain comprising a transcriptional activator. Binding of the antibody to the target antigen induces cleavage of the non-Notch force sensor cleavage domain at the proteolytic cleavage site, thereby releasing the intracellular domain The non-Notch force sensor cleavage domain is selected from the group consisting of: a von Willebrand Factor (vWF) cleavage domain, an amyloid-beta cleavage domain, a CD16 cleavage domain, a CD44 cleavage domain, a Delta cleavage domain, a cadherin cleavage domain, an ephrin-type receptor or ephrin ligand cleavage domain, a protocadherin cleavage domain, a filamin cleavage domain, a synthetic E cadherin cleavage domain, an interleukin-1 receptor type 2 (IL1R2) cleavage domain, a major prion protein (PrP) cleavage domain, a neuregulin cleavage domain and an adhesion-GPCR cleavage domain

In some cases, the proteolytic cleavage site is an ADAM family type protease cleavage site. In some cases, the ADAM family type protease cleavage site is an ADAM-13 type protease cleavage site. In some cases, the cleavable transmembrane domain comprises a γ-secretase cleavage site. In some cases, the cleavable transmembrane domain is a Notch transmembrane domain In some cases, the Notch transmembrane domain comprises a γ-secretase cleavage site. In some cases, the γ-secretase cleavage site is a Notch S3 proteolytic cleavage site. In some cases, the Notch intracellular signaling domain is a drosophila Notch intracellular signaling domain In some cases, the non-Notch force sensor cleavage domain is a vWF cleavage domain In some cases, the vWF cleavage domain comprises a vWF A2 domain or a variant thereof. In some cases, the antibody is a nanobody, a diabody, a triabody, or a minibody, a F(ab′)₂ fragment, a Fab fragment, a single chain variable fragment (scFv) or a single domain antibody (sdAb).

An A2 chimeric polypeptide comprises a non-Notch force sensor cleavage domain, e.g., a vWF cleavage domain, an amyloid-beta cleavage domain, a CD16 cleavage domain, a CD44 cleavage domain, a Delta cleavage domain, a cadherin cleavage domain, an ephrin-type receptor or ephrin ligand cleavage domain, a protocadherin (e.g., drosophila fat) cleavage domain, a filamin cleavage domain, an E cadherin cleavage domain, an interleukin-1 receptor type 2 (i.e., IL1R2) cleavage domain, a major prion protein (i.e., PrP) cleavage domain, a neuregulin cleavage domain, an adhesion-GPCR cleavage domain, and homologs and variants thereof.

### Cleavage Domains

An A2 chimeric polypeptide will generally include a force sensor cleavage domain that is not derived from a Notch polypeptide (i.e., a non-Notch force sensor cleavage domain) Such non-Notch force sensor cleavage domains will vary and may be derived from a force sensitive protein or homolog or variant thereof and will generally include at least one proteolytic cleavage site of the force sensitive protein.

Force sensor cleavage domains that may find use in an A2 chimeric polypeptides include vWF cleavage domains. Useful vWF cleavage domains will vary and may be derived from a vWF protein or homolog thereof and will generally include at least one proteolytic cleavage site of the vWF protein. Useful vWF proteolytic cleavage sites include ADAM family type protease cleavage sites, including e.g., ADAM-13 type protease cleavage sites. In some instances, a vWF polypeptide included in a chimeric polypeptide of the present disclosure may include a vWF A2 domain or a variant thereof. For example, in some instances, a vWF cleavage domain may include a mammalian vWF A2 domain, including but not limited to e.g., a human vWF A2 domain, a non-human primate vWF A2 domain, a rodent vWF A2 domain (e.g., a mouse vWF A2 domain, a rat vWF A2 domain, etc.) and the like. In some instances, a vWF cleavage domain may include a non-mammalian vWF A2 domain, including but not limited to e.g., an avian vWF A2 domain, a reptile vWF A2 domain, an amphibian vWF A2 domain, a fish vWF A2 domain, etc. Useful vWF A2 domains may include those vWF A2 domains that are naturally occurring or non-natural variants thereof, including e.g., domains having less than 100% sequence identity with a naturally occurring vWF A2 domain, including one or more of the domains provided herein, such as less than 100% but at least 40%, at least 50%, at least 60%, at least 70%, at least 75%, at least 80%, at least 85%, at least 90%, at least 95%, at least 96%, at least 97%, at least 98% or at least 99% sequence identity with a naturally occurring vWF A2 domain (including e.g., mammalian and/or non-mammalian vWF A2 domains).

Useful human vWF cleavage domains may include e.g., amino acids 1480-1678 of the human vWF protein of UniProtKB ID P04275 or NCBI RefSeq ID NP_000543.2:

PGLLGVSTLGPKRNSMVLDVAFVLEGSDKIGEADFNRSKEFMEEVIQRMDVGQDSIHVTVLQYS YMVTVEYPFSEAQSKGDILQRVREIRYQGGNRTNTGLALRYLSDHSFLVSQGDREQAPNLVYM VTGNPASDEIKRLPGDIQVVPIGVGPNANVQELERIGWPNAPILIQDFETLPREAPDLVLQRCCSG EGLQI (SEQ ID NO:58), or a polypeptide having less than 100% sequence identity with the provided sequence, including e.g., at least 99% sequence identity, at least 98%, at least 97%, at least 96%, at least 95%, at least 90%, at least 85%, at least 80%, at least 75%, at least 70%, at least 65% or at least 60% sequence identity with the provided sequence.

In some instances, a useful vWF cleavage domain may include the following amino acid sequence:

PGLLGVKKLGPKRNSMVLDVAFVLEGSDKIGEADFNRSKEFMEEVIQRMDVGQDSIHVTVLQY SYMVTVEYPFSEAQSKGDILQRVREIRYQGGNRTNTGLALRYLSDHSFLVSQGDREQAPNLVY MVTGNPASDEIKRLPGDIQVVPIGVGPNANVQELERIGWPNAPILIQDFETLPREAPDLVLQRCCS GEGLQI (SEQ ID NO:59), or a polypeptide having less than 100% sequence identity with the provided sequence, including e.g., at least 99% sequence identity, at least 98%, at least 97%, at least 96%, at least 95%, at least 90%, at least 85%, at least 80%, at least 75%, at least 70%, at least 65% or at least 60% sequence identity with the provided sequence.

Useful mouse vWF cleavage domains may include e.g., amino acids 1480-1678 of the mouse vWF protein of UniProtKB ID Q8CIZ8:

PGIAGISSPGPKRKSMVLDVVFVLEGSDEVGEANFNKSKEFVEEVIQRMDVSPDATRISVLQYSY TVTMEYAFNGAQSKEEVLRHVREIRYQGGNRTNTGQALQYLSEHSFSPSQGDRVEAPNLVYMV TGNPASDEIKRLPGDIQVVPIGVGPHANMQELERISRPIAPIFIRDFETLPREAPDLVLQTCCSKEG LQLP (SEQ ID NO:60), or a polypeptide having less than 100% sequence identity with the provided sequence, including e.g., at least 99% sequence identity, at least 98%, at least 97%, at least 96%, at least 95%, at least 90%, at least 85%, at least 80%, at least 75%, at least 70%, at least 65% or at least 60% sequence identity with the provided sequence.

Useful mouse vWF cleavage domains may include e.g., amino acids 183-381 of GenBank ID AAA82929.1:

PGIAGTLSPGPKRKSMVLDVVFVLEGSDEVGEANFNKSKEFVEEVIQRMDVSPDATRISVLQYS YTVTMEYAFNGAQSKEEVLRHVREIRYQGGNRTNTGQALQYLSEHSFSPSQGDRVEAPNLVYM VTGNPASDEIKRLPGDIQVVPIGVGPHANMQELERISRPIAPIFIRDFETLPREAPDLVLQTCCSKE GLQLP (SEQ ID NO:61), or a polypeptide having less than 100% sequence identity with the provided sequence, including e.g., at least 99% sequence identity, at least 98%, at least 97%, at least 96%, at least 95%, at least 90%, at least 85%, at least 80%, at least 75%, at least 70%, at least 65% or at least 60% sequence identity with the provided sequence.

Useful vWF cleavage domains will vary in length, including e.g., where the overall length of the vWF cleavage domain is 1000 amino acids or less, including e.g., 900 amino acids or less, 800 amino acids or less, 700 amino acids or less, 600 amino acids or less, 500 amino acids or less, 400 amino acids or less, 300 amino acids or less or 200 amino acids or less. In some instances, the subject vWF cleavage domain may range from less than 150 to more than 1000 amino acid in length, including but not limited to e.g., from 150 to 1000, from 150 to 900, from 150 to 800, from 150 to 700, from 150 to 600, from 150 to 500, from 150 to 400, from 150 to 350, from 150 to 300, from 150 to 275, from 150 to 250, from 150 to 225, from 150 to 200, or the like.

In some instances, a vWF cleavage domain may include sequence of a vWF protein in the N- and/or C-terminal direction adjacent to a vWF A2 domain, including up to 100 amino acids or more in the N- and/or C-terminal direction adjacent to the A2 domain, including but not limited to e.g., 100 amino acids or less, 90 amino acids or less, 80 amino acids or less, 70 amino acids or less, 60 amino acids or less, 50 amino acids or less, 40 amino acids or less, 30 amino acids or less, 20 amino acids or less, 10 amino acids or less, etc., in the N- and/or C-terminal direction adjacent to a vWF A2 domain

Force sensor cleavage domains that may find use in the instant chimeric polypeptides include amyloid-beta cleavage domains. Useful amyloid-beta cleavage domains will vary and may be derived from an amyloid-beta protein (e.g., amyloid-beta A4 protein, amyloid precursor protein (APP), etc.) or homolog thereof and will generally include at least one proteolytic cleavage site of the amyloid-beta protein. In some instances, an amyloid-beta polypeptide included in a chimeric polypeptide of the present disclosure may be a mammalian amyloid-beta cleavage domain or a variant thereof, including but not limited to e.g., human, non-human primate, rodent (e.g., mouse, rat, etc.), and the like amyloid-beta cleavage domains and homologs and variants thereof. Useful amyloid-beta cleavage domains may include those amyloid-beta cleavage domains that are naturally occurring or non-natural variants thereof, including e.g., domains having less than 100% sequence identity with a naturally occurring amyloid-beta cleavage domain, including one or more of the domains provided herein, such as less than 100% but at least 40%, at least 50%, at least 60%, at least 70%, at least 75%, at least 80%, at least 85%, at least 90%, at least 95%, at least 96%, at least 97%, at least 98% or at least 99% sequence identity with a naturally occurring amyloid-beta cleavage domain (including e.g., mammalian and/or non-mammalian amyloid-beta cleavage domains.

Useful amyloid-beta cleavage domains may include e.g., those derived from accession number RefSeq NP_001129603.1 or a homolog or variant thereof, including e.g.,:

(SEQ ID NO: 62)
MVSKGEEDNSDVWWGGADTDYADGSEDKVVEVAEEEEVAEVEEEEADDD
EDDEDGDEVEEEAEEPYEEATERTTSIATTTTTTTESVEEVVRVPTTAA
STPDAVDKYLETPGDENEHAHFQKAKERLEAKHRERMSQVMREWEEAER
QAKNLPKADKKAVIQHFQEKVESLEQEAANERQQLVETHMARVEAMLND
RRRLALENYITALQAVPPRPRHVFNMLKKYVRAEQKDRQHTLKHFEHVR
MVDPKKAAQIRSQVMTHLRVIYERMNQSLSLLYNVPAVAEEIQDEVDEL
LQKEQNYSDDVLANMISEPRISYGNDALMPSLTETKTTVELLPVNGEFS
LDDLQPWHSFGADSVPANTENEVEPVDARPAADRGLTTRPGSGLTNIKT
EEISEVNLDAEFRHDSGYEVHHQKLVFFAEDVGSNKGR,

or a polypeptide having less than 100% sequence identity with the preceding sequence or another sequence derived from the protein of the provided accession number, including e.g., at least 99% sequence identity, at least 98%, at least 97%, at least 96%, at least 95%, at least 90%, at least 85%, at least 80%, at least 75%, at least 70%, at least 65% or at least 60% sequence identity with one or more of the provided sequences.

Subject amyloid-beta cleavage domains may be derived from or include a portion of a sequence from a wide variety of amyloid-beta protein sequences.

Force sensor cleavage domains that may find use in the instant chimeric polypeptides include CD16 cleavage domains. Useful CD16 cleavage domains will vary and may be derived from a CD16 protein (e.g., low affinity immunoglobulin gamma Fc region receptor III protein) or homolog thereof and will generally include at least one proteolytic cleavage site of the CD16 protein. In some instances, a CD16 polypeptide included in a chimeric polypeptide of the present disclosure may be a mammalian CD16 cleavage domain or a variant thereof, including but not limited to e.g., human, non-human primate, rodent (e.g., mouse, rat, etc.), and the like CD16 cleavage domains and homologs and variants thereof. Useful CD16 cleavage domains may include those CD16 cleavage domains that are naturally occurring or non-natural variants thereof, including e.g., domains having less than 100% sequence identity with a naturally occurring CD16 cleavage domain, including one or more of the domains provided herein, such as less than 100% but at least 40%, at least 50%, at least 60%, at least 70%, at least 75%, at least 80%, at least 85%, at least 90%, at least 95%, at least 96%, at least 97%, at least 98% or at least 99% sequence identity with a naturally occurring CD16 cleavage domain (including e.g., mammalian CD16 cleavage domains).

Useful CD16 cleavage domains may include e.g., those derived from accession number RefSeq NP_001121065.1 or a homolog or variant thereof, including e.g.,:

GMRTEDLPKAVVFLEPQWYRVLEKDSVTLKCQGAYSPEDNSTQWFHNESLISSQASSYFIDAAT VDDSGEYRCQTNLSTLSDPVQLEVHIGWLLLQAPRWVFKEEDPIHLRCHSWKNTALHKVTYLQ NGKGRKYFHHNSDFYIPKATLKDSGSYFCRGLFGSKNVSSETVNITITQGLAVSTISSFFPPGYQV R (SEQ ID NO:63), or a polypeptide having less than 100% sequence identity with the preceding sequence or another sequence derived from the protein of the provided accession number, including e.g., at least 99% sequence identity, at least 98%, at least 97%, at least 96%, at least 95%, at least 90%, at least 85%, at least 80%, at least 75%, at least 70%, at least 65% or at least 60% sequence identity with one or more of the provided sequences.

Subject CD16 cleavage domains may be derived from or include a portion of a sequence from a wide variety of CD16 protein sequences.

Force sensor cleavage domains that may find use in the instant chimeric polypeptides include CD44 cleavage domains. Useful CD44 cleavage domains will vary and may be derived from a CD44 protein (e.g., CD44 antigen isoform a precursor) or homolog thereof and will generally include at least one proteolytic cleavage site of the CD44 protein. In some instances, a CD44 polypeptide included in a chimeric polypeptide of the present disclosure may be a mammalian CD44 cleavage domain or a variant thereof, including but not limited to e.g., human, non-human primate, rodent (e.g., mouse, rat, etc.), and the like CD44 cleavage domains and homologs and variants thereof. Useful CD44 cleavage domains may include those CD44 cleavage domains that are naturally occurring or non-natural variants thereof, including e.g., domains having less than 100% sequence identity with a naturally occurring CD44 cleavage domain, including one or more of the domains provided herein, such as less than 100% but at least 40%, at least 50%, at least 60%, at least 70%, at least 75%, at least 80%, at least 85%, at least 90%, at least 95%, at least 96%, at least 97%, at least 98% or at least 99% sequence identity with a naturally occurring CD44 cleavage domain (including e.g., mammalian and/or non-mammalian CD44 cleavage domains).

Useful CD44 cleavage domains may include e.g., those derived from accession number RefSeq NP_033981.2 or a homolog or variant thereof, including e.g.,:

PRHSKSHAAAQKQNNWIWSWFGNSQSTTQTQEPTTSATTALMTTPETPPKRQEAQNWFSWLFQ PSESKSHLHTTTKMPGTESNTNPTGWEPNEENEDETDKYPSFSGSGIDDDEDFISSTIASTPRVSA RTEDNQDWTQWKPNHSNPEVLLQTTTRMADIDRISTSAHGENWTPEPQPPFNNHEYQDEEETP HATSTTPNSTAEAAATQQETWFQNGWQGKNPPTPSEDSHVTEGTTASAHNNHPSQRITTQSQED VSWTDFFDPISHPMGQGHQTESKDTDSSHSTTLQPTAAPNTHLVEDLNRTGPLSVTTPQSHSQNF STLHGEPEEDENHPTTSILPSSTKSGAKDARRGGSLPTDTTTSVEGYTFQYPDTMENGTLFPVTP AKTEVFGETEVTLATDSNVNVDGSLPGDRDSSKDSRGSSRTVTHGSELAGHSSANQDSGVTTTS GPMRRPQIPER (SEQ ID NO:64), or a polypeptide having less than 100% sequence identity with the preceding sequence or another sequence derived from the protein of the provided accession number, including e.g., at least 99% sequence identity, at least 98%, at least 97%, at least 96%, at least 95%, at least 90%, at least 85%, at least 80%, at least 75%, at least 70%, at least 65% or at least 60% sequence identity with one or more of the provided sequences.

Force sensor cleavage domains that may find use in the instant chimeric polypeptides include Delta cleavage domains. Useful Delta cleavage domains will vary and may be derived from a Delta protein (e.g., Drosophila neurogenic locus protein delta) or homolog thereof and will generally include at least one proteolytic cleavage site of the Delta protein. In some instances, a Delta polypeptide included in a chimeric polypeptide of the present disclosure may be a mammalian Delta cleavage domain or a variant thereof, including but not limited to e.g., human, non-human primate, rodent (e.g., mouse, rat, etc.), and the like Delta cleavage domains and homologs and variants thereof. Useful Delta cleavage domains may include those Delta cleavage domains that are naturally occurring or non-natural variants thereof, including e.g., domains having less than 100% sequence identity with a naturally occurring Delta cleavage domain, including one or more of the domains provided herein, such as less than 100% but at least 40%, at least 50%, at least 60%, at least 70%, at least 75%, at least 80%, at least 85%, at least 90%, at least 95%, at least 96%, at least 97%, at least 98% or at least 99% sequence identity with a naturally occurring Delta cleavage domain (including e.g., mammalian and/or non-mammalian Delta cleavage domains).

Useful Delta cleavage domains may include e.g., those derived from accession number GenBank CAA29617.1 or a homolog or variant thereof, including e.g.,:

PRDEESYDSVTFDAHQYGATTQARADGLANAQVR (SEQ ID NO:65), or a polypeptide having less than 100% sequence identity with the preceding sequence or another sequence derived from the protein of the provided accession number, including e.g., at least 99% sequence identity, at least 98%, at least 97%, at least 96%, at least 95%, at least 90%, at least 85%, at least 80%, at least 75%, at least 70%, at least 65% or at least 60% sequence identity with one or more of the provided sequences.

Force sensor cleavage domains that may find use in an A2 chimeric polypeptide include cadherin cleavage domains. Useful cadherin cleavage domains will vary and may be derived from a cadherin protein (e.g., cadherin-1 preproprotein) or homolog thereof and will generally include at least one proteolytic cleavage site of the cadherin protein. In some instances, a cadherin polypeptide included in an A2 chimeric polypeptide may be a mammalian cadherin cleavage domain or a variant thereof, including but not limited to e.g., human, non-human primate, rodent (e.g., mouse, rat, etc.), and the like cadherin cleavage domains and homologs and variants thereof. Useful cadherin cleavage domains may include those cadherin cleavage domains that are naturally occurring or non-natural variants thereof, including e.g., domains having less than 100% sequence identity with a naturally occurring cadherin cleavage domain, including one or more of the domains provided herein, such as less than 100% but at least 40%, at least 50%, at least 60%, at least 70%, at least 75%, at least 80%, at least 85%, at least 90%, at least 95%, at least 96%, at least 97%, at least 98% or at least 99% sequence identity with a naturally occurring cadherin cleavage domain (including e.g., mammalian and/or non-mammalian cadherin cleavage domains).

Useful cadherin cleavage domains may include e.g., those derived from accession number RefSeq NP_033994.1 or a homolog or variant thereof, including e.g.,:

AEMDREDAEHVKNSTYVALIIATDDGSPIATGTGTLLLVLLDVNDNAPIPEPRNMQFCQRNPQP HIITILDPDLPPNTSPFTAELTHGASVNWTIEYNDAAQESLILQPRKDLEIGEYKIHLKLADNQNK DQVTTLDVHVCDCEGTVNNCMKAGIVAAGLQVR (SEQ ID NO:66), or a polypeptide having less than 100% sequence identity with the preceding sequence or another sequence derived from the protein of the provided accession number, including e.g., at least 99% sequence identity, at least 98%, at least 97%, at least 96%, at least 95%, at least 90%, at least 85%, at least 80%, at least 75%, at least 70%, at least 65% or at least 60% sequence identity with one or more of the provided sequences.

Force sensor cleavage domains that may find use in the instant chimeric polypeptides include ephrin-type receptor and ephrin ligand cleavage domains. Useful ephrin-type receptor and ephrin ligand cleavage domains will vary and may be derived from an ephrin-type receptor and ephrin ligand proteins (e.g., ephrin type-B receptor 2, ephrin-B2 precursor, ephrin-A2 precursor, etc.) or homolog thereof and will generally include at least one proteolytic cleavage site of the protein. In some instances, an ephrin-type receptor or ephrin ligand polypeptide included in an A2 chimeric polypeptide may be a mammalian ephrin-type receptor or ephrin ligand cleavage domain or a variant thereof, including but not limited to e.g., human, non-human primate, rodent (e.g., mouse, rat, etc.), and the like ephrin-type receptor or ephrin ligand cleavage domains and homologs and variants thereof. Useful ephrin-type receptor and ephrin ligand cleavage domains may include those ephrin-type receptor and ephrin ligand cleavage domains that are naturally occurring or non-natural variants thereof, including e.g., domains having less than 100% sequence identity with a naturally occurring ephrin-type receptor or ephrin ligand cleavage domain, including one or more of the domains provided herein, such as less than 100% but at least 40%, at least 50%, at least 60%, at least 70%, at least 75%, at least 80%, at least 85%, at least 90%, at least 95%, at least 96%, at least 97%, at least 98% or at least 99% sequence identity with a naturally occurring ephrin-type receptor or ephrin ligand cleavage domain (including e.g., mammalian and/or non-mammalian ephrin-type receptor and ephrin ligand cleavage domains).

Useful ephrin-type receptor and ephrin ligand cleavage domains may include e.g., those derived from accession numbers RefSeq NP_001277682.1, RefSeq NP_034241.2, RefSeq NP_031935.3, or a homolog or variant thereof, including e.g.,:

NGAIFQETLSGAESTSLVAARGSCIANAEEVDVPIKLYCNGDGEWLVPIGRCMCKAGFEAVENG TVCRGCPSGTFKANQGDEACTHCPINSRTTSEGATNCVCRNGYYRADLDPLDMPCTTIPSAPQA VISSVNETSLMLEWTPPRDSGGREDLVYNIICKSCGSGRGACTRCGDNVQYAPRQLGLTEPRIYI SDLLAHTQYTFEIQAVNGVTDQSPFSPQFASVNITTNQAAPSAVSIMHQVSRTVDSITLSWSQPD QPNGVILDYELQYYEKQELSEYNATAIKSPTNTVTVQGLKAGAIYVFQVRARTVAGYGRYSGK MYFQTMTEAEYQTSIKEKLPR (SEQ ID NO:67),
APSAVSIMHQVSRTVDSITLSWSQPDQPNGVILDYELQYYEKQELSEYNATAIKSPTNTVTVQGL KAGAIYVFQVRARTVAGYGRYSGKMYFQTMTEAEYQTSIKEKLPR (SEQ ID NO:68), MAMARSRRDSVWKYCWGLLMVLCRTAISRSIVLEPIYWNSSNSKFLPGQGLVLYPQIGDKLDII CPKVDSKTVGQYEYYKVYMVDKDQADRCTIKKENTPLLNCARPDQDVKFTIKFQEFSPNLWGL EFQKNKDYYIISTSNGSLEGLDNQEGGVCQTRAMKILMKVGQDAS SAGSARNHGPTRRPELEA GTNGRSSTTSPFVKPNPGSSTDGNSAGHSGNNLLGSEVALFAR (SEQ ID NO:69), VYVRPTNETLYEAPEPIFTSNSSCSGLGGCHLFLTTVPVLWSLLGSR (SEQ ID NO:70), or GQDASSAGSARNHGPTRRPELEAGTNGRSSTTSPFVKPNPGSSTDGNSAGHSGNNLLGSEVALF AR (SEQ ID NO:71) or a polypeptide having less than 100% sequence identity with the preceding sequence or another sequence derived from the protein of the provided accession number, including e.g., at least 99% sequence identity, at least 98%, at least 97%, at least 96%, at least 95%, at least 90%, at least 85%, at least 80%, at least 75%, at least 70%, at least 65% or at least 60% sequence identity with one or more of the provided sequences.

Force sensor cleavage domains that may find use in an A2 chimeric polypeptide include protocadherin cleavage domains. Useful protocadherin cleavage domains will vary and may be derived from a protocadherin protein (e.g., Drosophila fat) or homolog thereof and will generally include at least one proteolytic cleavage site of the protocadherin protein. In some instances, a protocadherin polypeptide included in an A2 chimeric polypeptide may be a mammalian protocadherin cleavage domain or a variant thereof, including but not limited to e.g., human, non-human primate, rodent (e.g., mouse, rat, etc.), and the like protocadherin cleavage domains and homologs and variants thereof. Useful protocadherin cleavage domains may include those protocadherin cleavage domains that are naturally occurring or non-natural variants thereof, including e.g., domains having less than 100% sequence identity with a naturally occurring protocadherin cleavage domain, including one or more of the domains provided herein, such as less than 100% but at least 40%, at least 50%, at least 60%, at least 70%, at least 75%, at least 80%, at least 85%, at least 90%, at least 95%, at least 96%, at least 97%, at least 98% or at least 99% sequence identity with a naturally occurring protocadherin cleavage domain (including e.g., mammalian and/or non-mammalian protocadherin cleavage domains).

Useful protocadherin cleavage domains may include e.g., those derived from accession number RefSeq NP_477497.1 or a homolog or variant thereof, including e.g.,:

DNQQMRERRAVSNFSTASQIYEAPKMLSMLFRTYKDQGQILYAATNQMFTSLSLREGRLVYYS KQHLTINMTVQETSTLNDGKWHNVSLFSESRSLRLIVDGRQVGDELDIAGVHDFLDPYLTILNV GGEAFVGCLANVTVNNELQPLNGSGSIFPEVRYHGKIESGCRGDIGQDAAQVADPLSIGFTLVIV FFVILVVAILGSYVIYRFR (SEQ ID NO:72), or a polypeptide having less than 100% sequence identity with the preceding sequence or another sequence derived from the protein of the provided accession number, including e.g., at least 99% sequence identity, at least 98%, at least 97%, at least 96%, at least 95%, at least 90%, at least 85%, at least 80%, at least 75%, at least 70%, at least 65% or at least 60% sequence identity with one or more of the provided sequences.

Force sensor cleavage domains that may find use in the instant chimeric polypeptides include filamin cleavage domains. Useful filamin cleavage domains will vary and may be derived from a filamin protein (e.g., filamin-A isoform 2) or homolog thereof and will generally include at least one proteolytic cleavage site of the filamin protein. In some instances, a filamin polypeptide included in a chimeric polypeptide of the present disclosure may be a mammalian filamin cleavage domain or a variant thereof, including but not limited to e.g., human, non-human primate, rodent (e.g., mouse, rat, etc.), and the like filamin cleavage domains and homologs and variants thereof. Useful filamin cleavage domains may include those filamin cleavage domains that are naturally occurring or non-natural variants thereof, including e.g., domains having less than 100% sequence identity with a naturally occurring filamin cleavage domain, including one or more of the domains provided herein, such as less than 100% but at least 40%, at least 50%, at least 60%, at least 70%, at least 75%, at least 80%, at least 85%, at least 90%, at least 95%, at least 96%, at least 97%, at least 98% or at least 99% sequence identity with a naturally occurring filamin cleavage domain (including e.g., mammalian and/or non-mammalian filamin cleavage domains).

Useful filamin cleavage domains may include e.g., those derived from accession number RefSeq NP_001104026.1 or a homolog or variant thereof, including e.g.,:

MACKMQIFVKTLTGKTITLEVEPSDTIENVKAKIQDKEGIPPDQQRLIFAGKQLEDGRTLSDYNIQ KESTLHLVLRLRGGELGGSGGSGEGRVKESITRRRRAPSVANVGSHSDLSLKIPEISIQDMTAQV TSPSGKTHEAEIVEGENHTYSIRFVPAEMGTHTVSVKYKGQHVPGSPFQFTVGPLGEGGAHKVR AGGPGLERAEAGVPAEFSIWTREAGAGGLAIAVEGPSKAEISFEDRKDGSSGVAYVVQEPGDYE VSVKFNEEHIPDSPFVVPVASPSSGGSGGTMQIFVKTLTGKTITLEVEPSDTIENVKAKIQDKEGIP PDQQRLIFAGKQLEDGRTLSDYNIQKESTLHLVLRLRGGKCLER (SEQ ID NO:73) or MACKMQIFVKTLTGKTITLEVEPSDTIENVKAKIQDKEGIPPDQQRLIFAGKQLEDGRTLSDYNIQ KESTLHLVLRLRGGELGGSGGPTFRSSLFLWVRPGGSGGSGPLGEGGAHKVRAGGPGLERAEA GVPAEFSIWTREAGAGGLAIAVEGPSKAEISFEDRKDGSCGVAYVVQEPGDYEVSVKFNEEHIP DSPFVVPVASPSSGGSGGTMQIFVKTLTGKTITLEVEPSDTIENVKAKIQDKEGIPPDQQRLIFAG KQLEDGRTLSDYNIQKESTLHLVLRLRGGKCLER (SEQ ID NO:74), or a polypeptide having less than 100% sequence identity with the preceding sequence or another sequence derived from the protein of the provided accession number, including e.g., at least 99% sequence identity, at least 98%, at least 97%, at least 96%, at least 95%, at least 90%, at least 85%, at least 80%, at least 75%, at least 70%, at least 65% or at least 60% sequence identity with one or more of the provided sequences.

Force sensor cleavage domains that may find use in an A2 chimeric polypeptide include E cadherin cleavage domains. Useful E cadherin cleavage domains will vary and may be derived from an E cadherin protein or homolog thereof or recombinant variants thereof (e.g., EcadTS) and will generally include at least one proteolytic cleavage site of the E cadherin protein. In some instances, an E cadherin polypeptide included in an A2 chimeric polypeptide may be a mammalian E cadherin cleavage domain or a variant thereof, including but not limited to e.g., human, non-human primate, rodent (e.g., mouse, rat, etc.), and the like E cadherin cleavage domains and homologs and variants thereof. Useful E cadherin cleavage domains may include those E cadherin cleavage domains that are naturally occurring or non-natural variants thereof, including e.g., domains having less than 100% sequence identity with a naturally occurring E cadherin cleavage domain, including one or more of the domains provided herein, such as less than 100% but at least 40%, at least 50%, at least 60%, at least 70%, at least 75%, at least 80%, at least 85%, at least 90%, at least 95%, at least 96%, at least 97%, at least 98% or at least 99% sequence identity with a naturally occurring E cadherin cleavage domain (including e.g., mammalian and/or non-mammalian E cadherin cleavage domains).

Useful E cadherin cleavage domains may include e.g., those derived from accession number GenBank AID22384.1 or a homolog or variant thereof, including e.g.,:

MVSKGEETTMGVIKPDMKIKLKMEGNVNGHAFVIEGEGEGKPYDGTNTINLEVKEGAPLPFSY DILTTAFAYGNRAFTKYPDDIPNYFKQSFPEGYSWERTMTFEDKGIVKVKSDISMEEDSFIYEIHL KGENFPPNGPVMQKKTTGWDASTERMYVRDGVLKGDVKHKLLLEGGGHHRVDFKTIYRAKK AVKLPDYHFVDHRIEILNHDKDYNKVTVYESAVARNSTDGMDELYKGPGGAGPGGAGPGGAG PGGAGPGGAGPGGAGPGGAGPGGAMVSKGEELFTGVVPILVELDGDVNGHKFSVSGEGEGDA TYGKLTLKLICTTGKLPVPWPTLVTTLGYGLQCFARYPDHMKQHDFFKSAMPEGYVQERTIFFK DDGNYKTRAEVKFEGDTLVNRIELKGIDFKEDGNILGHKLEYNYNSHNVYITADKQKNGIKANF KIRHNIEDGGVQLADHYQQNTPIGDGPVLLPDNHYLSYQSKLSKDPNEKRDHMVLLEFVTAAGI TLGMDELYK (SEQ ID NO:75), or a polypeptide having less than 100% sequence identity with the preceding sequence or another sequence derived from the protein of the provided accession number, including e.g., at least 99% sequence identity, at least 98%, at least 97%, at least 96%, at least 95%, at least 90%, at least 85%, at least 80%, at least 75%, at least 70%, at least 65% or at least 60% sequence identity with one or more of the provided sequences.

Force sensor cleavage domains that may find use in an A2 chimeric polypeptide include interleukin-1 receptor type 2 (i.e. IL1R2) cleavage domains. Useful IL1R2 cleavage domains will vary and may be derived from an IL1R2 protein (e.g., interleukin-1 receptor type 2 isoform 1 precursor) or homolog thereof and will generally include at least one proteolytic cleavage site of the IL1R2 protein. In some instances, an IL1R2 polypeptide included in A2 chimeric polypeptide may be a mammalian IL1R2 cleavage domain or a variant thereof, including but not limited to e.g., human, non-human primate, rodent (e.g., mouse, rat, etc.), and the like IL1R2 cleavage domains and homologs and variants thereof. Useful IL1R2 cleavage domains may include those IL1R2 cleavage domains that are naturally occurring or non-natural variants thereof, including e.g., domains having less than 100% sequence identity with a naturally occurring IL1R2 cleavage domain, including one or more of the domains provided herein, such as less than 100% but at least 40%, at least 50%, at least 60%, at least 70%, at least 75%, at least 80%, at least 85%, at least 90%, at least 95%, at least 96%, at least 97%, at least 98% or at least 99% sequence identity with a naturally occurring IL1R2 cleavage domain (including e.g., mammalian and/or non-mammalian IL1R2 cleavage domains).

Useful IL1R2 cleavage domains may include e.g., those derived from accession number RefSeq NP_004624.1 or a homolog or variant thereof, including e.g.,:

AARSCRFRGRHYKREFRLEGEPVALRCPQVPYWLWASVSPRINLTWHKNDSARTVPGEEETRM WAQDGALWLLPALQEDSGTYVCTTRNASYCDKMSIELRVFENTDAFLPFISYPQILTLSTSGVLV CPDLSEFTRDKTDVKIQWYKDSLLLDKDNEKFLSVRGTTHLLVHDVALEDAGYYRCVLTFAHE GQQYNITRSIELRIKKKKEETIPVIISPLKTISASLGSRLTIPCKVFLGTGTPLTTMLWWTANDTHIE SAYPGGRVTEGPRQEYSENNENYIEVPLIFDPVTREDLHMDFKCVVHNTLSFQTLRTTVKEASST FSGR (SEQ ID NO:76), or a polypeptide having less than 100% sequence identity with the preceding sequence or another sequence derived from the protein of the provided accession number, including e.g., at least 99% sequence identity, at least 98%, at least 97%, at least 96%, at least 95%, at least 90%, at least 85%, at least 80%, at least 75%, at least 70%, at least 65% or at least 60% sequence identity with one or more of the provided sequences.

Force sensor cleavage domains that may find use in an A2 chimeric polypeptide include major prion protein (i.e. PrP) cleavage domains. Useful PrP cleavage domains will vary and may be derived from a PrP protein (e.g., major prion protein precursor) or homolog thereof and will generally include at least one proteolytic cleavage site of the PrP protein. In some instances, a PrP polypeptide included in an A2 chimeric polypeptide may be a mammalian PrP cleavage domain or a variant thereof, including but not limited to e.g., human, non-human primate, rodent (e.g., mouse, rat, etc.), and the like PrP cleavage domains and homologs and variants thereof. Useful PrP cleavage domains may include those PrP cleavage domains that are naturally occurring or non-natural variants thereof, including e.g., domains having less than 100% sequence identity with a naturally occurring PrP cleavage domain, including one or more of the domains provided herein, such as less than 100% but at least 40%, at least 50%, at least 60%, at least 70%, at least 75%, at least 80%, at least 85%, at least 90%, at least 95%, at least 96%, at least 97%, at least 98% or at least 99% sequence identity with a naturally occurring PrP cleavage domain (including e.g., mammalian and/or non-mammalian PrP cleavage domains).

Useful PrP cleavage domains may include e.g., those derived from accession number RefSeq NP_035300.1 or a homolog or variant thereof, including e.g.,:

KRPKPGGWNTGGSRYPGQGSPGGNRYPPQGGTWGQPHGGGWGQPHGGSWGQPHGGSWGQP HGGGWGQGGGTHNQWNKPSKPKTNLKHVAGAAAAGAVVGGLGGYMLGSAMSRPMIHFGND WEDRYYRENMYRYPNQVYYRPVDQYSNQNNFVHDCVNITIKQHTVTTTTKGENFTETDVKM MERVVEQMCVTQYQKESQAYYDGRRSSSTVLFSSPPVILLISFLIFLIVGR (SEQ ID NO:77), or a polypeptide having less than 100% sequence identity with the preceding sequence or another sequence derived from the protein of the provided accession number, including e.g., at least 99% sequence identity, at least 98%, at least 97%, at least 96%, at least 95%, at least 90%, at least 85%, at least 80%, at least 75%, at least 70%, at least 65% or at least 60% sequence identity with one or more of the provided sequences.

Force sensor cleavage domains that may find use in an A2 chimeric polypeptide include neuregulin cleavage domains. Useful neuregulin cleavage domains will vary and may be derived from a neuregulin protein (e.g., pro-neuregulin-1, membrane-bound isoform isoform 111-3, neuregulin Nrg1 (type III), etc.) or homolog thereof and will generally include at least one proteolytic cleavage site of the neuregulin protein. In some instances, a neuregulin polypeptide included in an A2 chimeric polypeptide may be a mammalian neuregulin cleavage domain or a variant thereof, including but not limited to e.g., human, non-human primate, rodent (e.g., mouse, rat, etc.), and the like neuregulin cleavage domains and homologs and variants thereof. Useful neuregulin cleavage domains may include those neuregulin cleavage domains that are naturally occurring or non-natural variants thereof, including e.g., domains having less than 100% sequence identity with a naturally occurring neuregulin cleavage domain, including one or more of the domains provided herein, such as less than 100% but at least 40%, at least 50%, at least 60%, at least 70%, at least 75%, at least 80%, at least 85%, at least 90%, at least 95%, at least 96%, at least 97%, at least 98% or at least 99% sequence identity with a naturally occurring neuregulin cleavage domain (including e.g., mammalian and/or non-mammalian neuregulin cleavage domains).

Useful neuregulin cleavage domains may include e.g., those derived from accession number RefSeq NP_001309136.1 or a homolog or variant thereof, including e.g.,:

GDRCQNYVMASFYKHLGIEFMEAEELYQKRVLTITGICIAR (SEQ ID NO:78), or a polypeptide having less than 100% sequence identity with the preceding sequence or another sequence derived from the protein of the provided accession number, including e.g., at least 99% sequence identity, at least 98%, at least 97%, at least 96%, at least 95%, at least 90%, at least 85%, at least 80%, at least 75%, at least 70%, at least 65% or at least 60% sequence identity with one or more of the provided sequences.

Force sensor cleavage domains that may find use in an A2 chimeric polypeptide include adhesion-GPCR cleavage domains. Useful adhesion-GPCR cleavage domains will vary and may be derived from an adhesion-GPCR protein (e.g., Drosophila Flamingo) or homolog thereof and will generally include at least one proteolytic cleavage site of the adhesion-GPCR protein. In some instances, an adhesion-GPCR polypeptide included in an A2 chimeric polypeptide may be a mammalian adhesion-GPCR cleavage domain or a variant thereof, including but not limited to e.g., human, non-human primate, rodent (e.g., mouse, rat, etc.), and the like adhesion-GPCR cleavage domains and homologs and variants thereof. Useful adhesion-GPCR cleavage domains may include those adhesion-GPCR cleavage domains that are naturally occurring or non-natural variants thereof, including e.g., domains having less than 100% sequence identity with a naturally occurring adhesion-GPCR cleavage domain, including one or more of the domains provided herein, such as less than 100% but at least 40%, at least 50%, at least 60%, at least 70%, at least 75%, at least 80%, at least 85%, at least 90%, at least 95%, at least 96%, at least 97%, at least 98% or at least 99% sequence identity with a naturally occurring adhesion-GPCR cleavage domain (including e.g., mammalian and/or non-mammalian adhesion-GPCR cleavage domains).

Useful adhesion-GPCR cleavage domains may include e.g., those derived from accession number GenBank BAA84069.1 or a homolog or variant thereof, including e.g.,:

### PRNPQCVRWNSFTNRWTRLGCQTEIPDFDGDFNPAAQQAILVNCSCTHISSYAVIVDVIDPEDIPE PSLLVQR (SEQ ID NO:79) or

ITYPSEQMQQSEQVVYRSLGSPHLAQPIKLQMWLDVDSARFGPRSNPQCVRWNSFTNRWTRLG CQTEIPDFDGDFNPAAQQAILVNCSCTHISSYAVIVDVIDPEDIPEPSLLVQR (SEQ ID NO:80), or a polypeptide having less than 100% sequence identity with the preceding sequence or another sequence derived from the protein of the provided accession number, including e.g., at least 99% sequence identity, at least 98%, at least 97%, at least 96%, at least 95%, at least 90%, at least 85%, at least 80%, at least 75%, at least 70%, at least 65% or at least 60% sequence identity with one or more of the provided sequences.

Force sensor cleavage domains that may find use in an A2 chimeric polypeptide include synthetic cleavage domains, including e.g., flagellin-derived cleavage domains. Useful flagellin-derived cleavage domains will vary and may be derived from a flagellin protein or homolog thereof and will generally include at least one proteolytic cleavage site. Useful synthetic cleavage domains may include, e.g., PRGPGGAGPGGAGPGGAGPGGAGPGGAGPGGAGPGGAGPGGARR (SEQ ID NO:81) including e.g., domains having less than 100% sequence identity with synthetic cleavage domain, including one or more of the domains provided herein, such as less than 100% but at least 40%, at least 50%, at least 60%, at least 70%, at least 75%, at least 80%, at least 85%, at least 90%, at least 95%, at least 96%, at least 97%, at least 98% or at least 99% sequence identity with a naturally occurring synthetic cleavage domain (including e.g., the synthetic domain provided above).

The cleavage domains (including e.g., those provided above) may be included in an A2 chimeric polypeptide at any convenient and appropriate location that may vary, e.g., depending on the length of the cleavage domain, the inclusion or exclusion of additional domains (i.e., domains besides the cleavage domain) of the protein from which the cleavage domain is derived in the chimeric polypeptide, the presence or absence of other domains, e.g., as described herein, within the A2 chimeric polypeptide, and the like. In some embodiments, the cleavage domain may be positioned within the A2 chimeric polypeptide essentially as described for the vWF cleavage domain(s) described herein. In some embodiments, a cleavage domain may be inserted within the A2 chimeric polypeptide following a Notch domain, e.g., following and/or adjacent to a PPANVKYV (SEQ ID NO:82) of a Notch domain), or the like. Useful force sensor cleavage domains will vary in length, including e.g., where the overall length of the force sensor cleavage domain is 1000 amino acids or less, including e.g., 900 amino acids or less, 800 amino acids or less, 700 amino acids or less, 600 amino acids or less, 500 amino acids or less, 400 amino acids or less, 300 amino acids or less, 200 amino acids or less, 100 amino acids or less or 50 amino acids or less. In some instances, the subject force sensor cleavage domain may range from less than 40 to more than 1000 amino acid in length, including but not limited to e.g., from 40 to 1000, from 50 to 1000, from 75 to 1000, from 100 to 1000, from 125 to 1000, from 150 to 1000, from 150 to 900, from 150 to 800, from 150 to 700, from 150 to 600, from 150 to 500, from 150 to 400, from 150 to 350, from 150 to 300, from 150 to 275, from 150 to 250, from 150 to 225, from 150 to 200, from 40 to 900, from 40 to 800, from 40 to 700, from 40 to 600, from 40 to 500, from 40 to 400, from 40 to 350, from 40 to 300, from 40 to 275, from 40 to 250, from 40 to 225, from 40 to 200, from 40 to 100 or the like.

In some instances, a force sensor cleavage domain may include sequence of a force sensitive protein in the N- and/or C-terminal direction adjacent to a force sensor cleavage domain, including up to 100 amino acids or more in the N- and/or C-terminal direction adjacent to the force sensor cleavage domain, including but not limited to e.g., 100 amino acids or less, 90 amino acids or less, 80 amino acids or less, 70 amino acids or less, 60 amino acids or less, 50 amino acids or less, 40 amino acids or less, 30 amino acids or less, 20 amino acids or less, 10 amino acids or less, etc., in the N- and/or C-terminal direction adjacent to a force sensor cleavage domain

### Transmembrane Domains

An A2 chimeric polypeptide will generally include a transmembrane domain Useful transmembrane domains include those having a proteolytic cleavage site (i.e., cleavable transmembrane domains). Proteolytic cleavage of a cleavable transmembrane domain of an A2 chimeric polypeptide will generally be prevented prior to cleavage of the chimeric polypeptide at the force sensor cleavage domain. Put another way, within an A2 chimeric polypeptide, cleavage at a cleavable transmembrane domain cleavage site may be blocked, e.g., blocked by one or more ectodomains of the chimeric polypeptide, until the chimeric polypeptide is cleaved at a proteolytic cleavage site within the force sensor cleavage domain Thus, cleavage of an A2 chimeric polypeptide at a proteolytic cleavage site within the force sensor cleavage domain may thereby expose a cleavage site of the cleavable transmembrane domain, i.e., exposing an otherwise inaccessible transmembrane domain cleavage site to cleavage by a protease. The process whereby removal of one or more ectodomains is required for cleavage of a cleavable transmembrane domain may also be referred to as ectodomain shedding. As such, in some instances, ectodomain shedding by cleavage at a force sensor cleavage domain may provide for subsequent cleavage at a transmembrane domain cleavage site.

Various cleavable transmembrane domains may find use in an A2 chimeric polypeptide. For example, in some instances, useful cleavable transmembrane domains include those having, either naturally or artificially, a y-secretase cleavage site. Substrates of y-secretase include e.g., Alcadein α, Alcadein γ (calsyntenin), APLP1, APLP2, ApoER2, APP, AβPP, Betacellulin (BTC), Betaglycan, CD43, CD44, CSF1R, CX3CL1 (fractalkine), CXCL16, DCC, Deltal, Desmoglein-2, DNER, Dystroglycan, E-cadherin, EpCAM, EphA4, EphB2 EphrinB1, EphrinB2, ErbB4, GHR, HLA, HLA-A2, IFNaR2, IGF-1R, IL-1R1, IL-1R2, IL6R, IR, Ire1β, Ire1α, Jagged2, KCNE1, KCNE2, KCNE3, KCNE4, Klotho, L1, LAR, LRP1 (LDLR), LRP1B, LRP2 (megalin), LRP6, MUC1, Nav-β1, Nav-β2, Nav-β3, Nav-β4, N-cadherin, Nectin-1α, Neuregulin-1, Neuregulin-2, Notch1, Notch2, Notch3, Notch4, NPR-C, NRADD, p75-NTR, PAM, PLXDC2, Polyductin (PKHD1), Protocadherin-α4 (Pcdh-α4), Protocadherin-γ-C3 (Pcdh-γC3), PTP-LAR, Ptprz, RAGE, ROBO1, RPTPκ, RPTPμ, SorC3, SorCS1b, SorLA (LR11), Sortilin, Syndecan-1, Syndecan-2, Syndecan-3, Tie1, Tyrosinase, TYRP1, TYRP2, Vasorin, VE-cadherin, VEGF-R1, VGSC beta2, VLDLR, as well as those described in Bed & Sanders (Cell Mol Life Sci. (2008) 65(9):1311-1334) and Haapasalo & Kovacs (J Alzheimers Dis. (2011) 25(1):3-28); the disclosures of which are incorporated herein by reference in their entirety.

Useful transmembrane domains include but are not limited to Notch transmembrane domains, including e.g., invertebrate and vertebrate Notch transmembrane domains, including e.g., insect (e.g., drosophila) Notch transmembrane domains, mammalian (e.g., human, non-human primate, rodent (e.g., mouse, rat, etc.), etc.) Notch transmembrane domains, and the like. Notch transmembrane domains are generally cleavable transmembrane domains, as described herein, and may, e.g., include a y-secretase cleavage site, including natural and modified y-secretase cleavage sites, including e.g., a Notch S3 proteolytic cleavage site.

Useful Notch transmembrane domains include but are not limited to e.g., Notch 1, Notch 2, Notch 3 and Notch 4 transmembrane domains. Non-limiting examples of Notch transmembrane domains include but are not limited to e.g., FMYVAAAAFVLLFFVGCGVLL (SEQ ID NO: 83), LLYLLAVAVVIILFIILLGVI (SEQ ID NO:84), LPLLVAGAVLLLVILVLGVMV (SEQ ID NO:85), PVLCSPVAGVILLALGALLVL (SEQ ID NO:86), LMYVAAAAFVLLFFVGCGVLL (SEQ ID NO:87), LLYLLAVAVVIILFFILLGVI (SEQ ID NO:88), LLPLLVAGAVFLLIIFILGVM (SEQ ID NO:89), PILCSPVVGVLLLALGALLVL (SEQ ID NO:90), LHLMYVAAAAFVLLFFVGCGVLL (SEQ ID NO:91), LLYLLAVAVVIILFLILLGVI (SEQ ID NO:92), LPLLVAGAVFLLVIFVLGVMV (SEQ ID NO:93), and variants thereof.

A Notch transmembrane domain or a portion thereof utilized in an A2 chimeric polypeptide may include an S3 cleavage site (i.e., a gamma-secretase cleavage site). As such, an S3 proteolytic cleavage site can be located within the TM domain The S3 proteolytic cleavage site may be cleaved by gamma-secretase (y-secretase). A γ-secretase cleavage site can comprise a Gly-Val dipeptide sequence, where the enzyme cleaves between the Gly and the Val. For example, in some cases, an S3 proteolytic cleavage site has the amino acid sequence VGCGVLLS (SEQ ID NO:31), where cleavage occurs between the “GV” sequence. In some cases, an S3 proteolytic cleavage site comprises the amino acid sequence GCGVLLS (SEQ ID NO:32).

### Extracellular Antigen-Binding Domains

An A2 chimeric polypeptide comprises an antibody specific for a target antigen. The antibody can be any antigen-binding antibody-based polypeptide, a wide variety of which are known in the art. In some instances, the antibody is a monoclonal antibody, a single chain Fv (scFv), a Fab, etc. Other suitable antibodies include, e.g., cAb VHH (camelid antibody variable domains) and humanized versions, IgNAR VH (shark antibody variable domains) and humanized versions, sdAb VH (single domain antibody variable domains) and “camelized” antibody variable domains.

In some cases, the antigen-binding domain (antibody) is specific for a cancer antigen, i.e., an antigen expressed by (synthesized by) a neoplasia or cancer cell, i.e., a cancer cell associated antigen or a cancer (or tumor) specific antigen.

A cancer cell associated antigen can be an antigen associated with, e.g., a breast cancer cell, a B cell lymphoma, a pancreatic cancer, a Hodgkin lymphoma cell, an ovarian cancer cell, a prostate cancer cell, a mesothelioma, a lung cancer cell (e.g., a small cell lung cancer cell), a non-Hodgkin B-cell lymphoma (B-NHL) cell, an ovarian cancer cell, a prostate cancer cell, a mesothelioma cell, a lung cancer cell (e.g., a small cell lung cancer cell), a melanoma cell, a chronic lymphocytic leukemia cell, an acute lymphocytic leukemia cell, a neuroblastoma cell, a glioma, a glioblastoma, a medulloblastoma, a colorectal cancer cell, etc. A cancer cell associated antigen may also be expressed by a non-cancerous cell.

A cancer cell specific antigen can be an antigen specific for cancer and/or a particular type of cancer or cancer cell including e.g., a breast cancer cell, a B cell lymphoma, a pancreatic cancer, a Hodgkin lymphoma cell, an ovarian cancer cell, a prostate cancer cell, a mesothelioma, a lung cancer cell (e.g., a small cell lung cancer cell), a non-Hodgkin B-cell lymphoma (B-NHL) cell, an ovarian cancer cell, a prostate cancer cell, a mesothelioma cell, a lung cancer cell (e.g., a small cell lung cancer cell), a melanoma cell, a chronic lymphocytic leukemia cell, an acute lymphocytic leukemia cell, a neuroblastoma cell, a glioma, a glioblastoma, a medulloblastoma, a colorectal cancer cell, etc. A cancer (or tumor) specific antigen is generally not expressed by non-cancerous cells (or non-tumor cells). In some instances, a cancer (or tumor) specific antigen may be minimally expressed by one or more non-cancerous cell types (or non-tumor cell types). By “minimally expressed” is meant that the level of expression, in terms of either the per-cell expression level or the number of cells expressing, minimally, insignificantly or undetectably results in binding of the specific binding member to non-cancerous cells expressing the antigen.

An A2 chimeric polypeptide may, in some cases, target a surface expressed antigen. As used herein the term “surface expressed antigen” generally refers to antigenic proteins that are expressed at least partially extracellularly such that at least a portion of the protein is exposed outside the cells and available for binding with a binding partner. Essentially any surface expressed protein may find use as a target of an A2 chimeric polypeptide. Non-limiting examples of surface expressed antigens include but are not limited to e.g., CD19, CD20, CD30, CD38, Her2/neu, ERBB2, CA125, MUC-1, prostate-specific membrane antigen (PSMA), CD44 surface adhesion molecule, mesothelin, carcinoembryonic antigen (CEA), epidermal growth factor receptor (EGFR), EGFRvIII, vascular endothelial growth factor receptor-2 (VEGFR2), high molecular weight-melanoma associated antigen (HMW-MAA), IL-13R-a2, GD2, and the like. Surface expressed antigens that may be targeted also include but are not limited to e.g., those specifically targeted in conventional cancer therapies, including e.g., those targets of the targeted cancer therapeutics described herein.

In some instances, the antibody of an A2 chimeric polypeptide may target a cancer-associated antigen. In some instances, the antibody is specific for a cancer associated antigen. Non-limiting examples of cancer associated antigens include but are not limited to e.g., CD19, CD20, CD38, CD30, Her2/neu, ERBB2, CA125, MUC-1, prostate-specific membrane antigen (PSMA), CD44 surface adhesion molecule, mesothelin, carcinoembryonic antigen (CEA), epidermal growth factor receptor (EGFR), EGFRvIII, vascular endothelial growth factor receptor-2 (VEGFR2), high molecular weight-melanoma associated antigen (HMW-MAA), MAGE-A1, IL-13R-a2, GD2, and the like. Cancer-associated antigens also include, e.g., 4-1BB, 5T4, adenocarcinoma antigen, alpha-fetoprotein, BAFF, B-lymphoma cell, C242 antigen, CA-125, carbonic anhydrase 9 (CA-IX), C-MET, CCR4, CD152, CD19, CD20, CD200, CD22, CD221, CD23 (IgE receptor), CD28, CD30 (TNFRSF8), CD33, CD4, CD40, CD44 v6, CD51, CD52, CD56, CD74, CD80, CEA, CNT0888, CTLA-4, DRS, EGFR, EpCAM, CD3, FAP, fibronectin extra domain-B, folate receptor 1, GD2, GD3 ganglioside, glycoprotein 75, GPNMB, HER2/neu, HGF, human scatter factor receptor kinase, IGF-1 receptor, IGF-I, IgG1, L1-CAM, IL-13, IL-6, insulin-like growth factor I receptor, integrin α5B1, integrin αvβ3, MORAb-009, MS4A1, MUC1, mucin CanAg, N-glycolylneuraminic acid, NPC-1C, PDGF-R α, PDL192, phosphatidylserine, prostatic carcinoma cells, RANKL, RON, ROR1, SCH 900105, SDC1, SLAMF7, TAG-72, tenascin C, TGF beta 2, TGF-β, TRAIL-R1, TRAIL-R2, tumor antigen CTAA16.88, VEGF-A, VEGFR-1, VEGFR2, and vimentin.

The antibody portion of an A2 chimeric polypeptide can specifically bind an antigen that is associated with an inflammatory disease. Non-limiting examples of antigens associated with inflammatory disease include, e.g., AOC3 (VAP-1), CAM-3001, CCL11 (eotaxin-1), CD125, CD147 (basigin), CD154 (CD40L), CD2, CD20, CD23 (IgE receptor), CD25 (α chain of IL-2 receptor), CD3, CD4, CDS, IFN-α, IFN-γ, IgE, IgE Fc region, IL-1, IL-12, IL-23, IL-13, IL-17, IL-17A, IL-22, IL-4, IL-5, IL-5, IL-6, IL-6 receptor, integrin α4, integrin α4β7, LFA-1 (CD11a), myostatin, OX-40, scleroscin, SOST, TGF beta 1, TNF-α, and VEGF-A.

The antibody portion of an A2 chimeric polypeptide can specifically bind an autoantigen.

### Generating a Synthetic Immune Cell

A synthetic immune cell of the present disclosure can be generated using well-established methods. As noted above, a synthetic immune cell of the present disclosure is a genetically modified immune cell that is genetically modified with one or more nucleic acids comprising nucleotide sequences encoding: a) a chimeric polypeptide comprising: i) an antibody specific for a target antigen; and ii) a binding triggered transcriptional activator; and b) a cytokine or proliferation-inducing polypeptide that increases proliferation and/or activity of an effector immune cell, where the nucleotide sequence encoding the cytokine or proliferation-inducing polypeptide is operably linked to a transcriptional control element responsive to the transcriptional activator.

The one or more nucleic acids can be expression vectors. Suitable expression vectors include, but are not limited to, viral vectors (e.g. viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., Hum Gene Ther 5:1088 1097, 1999; WO 94/12649, WO 93/03769; WO 93/19191; WO 94/28938; WO 95/11984 and WO 95/00655); adeno-associated virus (see, e.g., Ali et al., Hum Gene Ther 9:81 86, 1998, Flannery et al., PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641 648, 1999; Ali et al., Hum Mol Genet 5:591 594, 1996; Srivastava in WO 93/09239, Samulski et al., J. Vir. (1989) 63:3822-3828; Mendelson et al., Virol. (1988) 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and the like. In some cases, the vector is a lentivirus vector. Also suitable are transposon-mediated vectors, such as piggyback and sleeping beauty vectors.

In some embodiments, the recombinant expression vector is a viral construct, e.g., a recombinant adeno-associated virus (AAV) construct, a recombinant adenoviral construct, a recombinant lentiviral construct, a recombinant retroviral construct, a recombinant lentiviral construct, etc. In some cases, a nucleic acid comprising a nucleotide sequence encoding a chimeric receptor polypeptide is a recombinant lentivirus vector. In some cases, a nucleic acid comprising a nucleotide sequence encoding a chimeric receptor polypeptide is a recombinant AAV vector. In some cases, a nucleic acid comprising a nucleotide sequence encoding a cytokine is a recombinant lentivirus vector. In some cases, a nucleic acid comprising a nucleotide sequence encoding a cytokine is a recombinant AAV vector.

The present disclosure provides a composition comprising: a) a first recombinant expression vector comprising a nucleotide encoding a chimeric polypeptide, where the chimeric polypeptide comprises: i) an antibody specific for a target antigen; and ii) a binding triggered transcriptional activator; and b) a second recombinant expression vector comprising a nucleotide encoding a cytokine or proliferation-inducing polypeptide that increases proliferation and/or activity of an effector immune cell, where the nucleotide sequence encoding the cytokine or proliferation-inducing polypeptide is operably linked to a transcriptional control element responsive to the transcriptional activator. In some cases, the chimeric polypeptide is a chimeric Notch polypeptide, as described above. In some cases, the cytokine is a variant IL-2 polypeptide, as described above.

An immune cell is genetically modified with one or more nucleic acids comprising nucleotide sequences encoding the chimeric polypeptide and the cytokine, where the genetic modification is accomplished by introducing into the immune cell the one or more nucleic acids. Methods of introducing a nucleic acid into a host cell (e.g., an immune cell) are known in the art and include, e.g., electroporation, transduction, transfection, etc.

### Compositions

The present disclosure provides a composition comprising: a) a synthetic immune cell as described above; and b) a second immune cell, where the second immune cell is a cytotoxic T cell (CTL). As described above, a synthetic immune cell is a genetically modified, in vitro immune cell, wherein the immune cell is genetically modified with one or more nucleic acids comprising nucleotide sequences encoding: a) a chimeric polypeptide comprising: i) an antibody specific for a target antigen; and ii) a binding triggered transcriptional activator; and b) a cytokine or proliferation-inducing polypeptide that increases proliferation and/or activity of an effector immune cell, where the nucleotide sequence encoding the cytokine or proliferation-inducing polypeptide is operably linked to a transcriptional control element responsive to the transcriptional activator.

In some cases, the second immune cell is a CTL that is genetically modified to express on its surface an exogenous T-cell receptor (TCR), wherein the exogenous TCR is specific for a target antigen. In some cases, the second immune cell is a CTL that is genetically modified to express on its surface a chimeric antigen receptor (CAR), wherein the CAR is specific for a target antigen. In some cases, the second immune cell is a CTL that is genetically modified to express a bispecific T-cell engager (BiTE), wherein the BiTE comprises: i) a first antigen-binding region specific for CD3; and ii) a second antigen-binding region specific for a target antigen other than CD3. In some cases, the target antigen to which the TCR, the CAR, or the BiTE binds is a different antigen than the antigen to which the antibody present in the chimeric polypeptide of the synthetic immune cell binds. In some cases, the target antigen to which the TCR, the CAR, or the BiTE binds is the same antigen as the antigen to which the antibody present in the chimeric polypeptide of the synthetic immune cell binds. In some cases, the TCR, the CAR, or the BiTE is specific for a cancer-associated antigen, and the antibody present in the chimeric polypeptide is specific for a cancer-associated antigen.

In some cases, the second immune cell is a CTL that is genetically modified with one or more nucleic acids comprising nucleotide sequences encoding a CAR. The terms “chimeric antigen receptor” and “CAR”, used interchangeably herein, refer to artificial multi-module molecules capable of triggering or inhibiting the activation of an immune cell which generally but not exclusively comprise an extracellular domain (e.g., a ligand/antigen binding domain), a transmembrane domain and one or more intracellular signaling domains. The term CAR is not limited specifically to CAR molecules but also includes CAR variants. CAR variants include split CARs wherein the extracellular portion (e.g., the ligand binding portion) and the intracellular portion (e.g., the intracellular signaling portion) of a CAR are present on two separate molecules. CAR variants also include ON-switch CARs which are conditionally activatable CARs, e.g., comprising a split CAR wherein conditional hetero-dimerization of the two portions of the split CAR is pharmacologically controlled (e.g., as described in PCT publication no. WO 2014/127261 and US Patent Application No. 2015/0368342, the disclosures of which are incorporated herein by reference in their entirety). CAR variants also include bispecific CARs, which include a secondary CAR binding domain that can either amplify or inhibit the activity of a primary CAR. CAR variants also include inhibitory chimeric antigen receptors (iCARs) which may, e.g., be used as a component of a bispecific CAR system, where binding of a secondary CAR binding domain results in inhibition of primary CAR activation. CAR molecules and derivatives thereof (i.e., CAR variants) are described, e.g., in PCT Application No. US2014/016527; Fedorov et al. Sci Transl Med (2013); 5(215):215ra172; Glienke et al. Front Pharmacol (2015) 6:21; Kakarla & Gottschalk 52 Cancer J (2014) 20(2):151-5; Riddell et al. Cancer J (2014) 20(2):141-4; Pegram et al. Cancer J (2014) 20(2):127-33; Cheadle et al. Immunol Rev (2014) 257(1):91-106; Barrett et al. Annu Rev Med (2014) 65:333-47; Sadelain et al. Cancer Discov (2013) 3(4):388-98; Cartellieri et al., J Biomed Biotechnol (2010) 956304; the disclosures of which are incorporated herein by reference in their entirety. Useful CARs also include the anti-CD19-4-1BB-CD3ζ CAR expressed by lentivirus loaded CTL019 (Tisagenlecleucel-T) CAR-T cells as commercialized by Novartis (Basel, Switzerland).

In some cases, the CAR comprises: a) an extracellular domain comprising an antigen-binding domain (e.g., an antibody, such as a scFv or a nanobody); b) a transmembrane region; and c) an intracellular signaling domain In some cases, the intracellular signaling domain comprises: i) a signaling domain from the zeta chain of human CD3; and ii) one or more costimulatory polypeptides. In some cases, the one or more costimulatory polypeptides is selected from CD28, 4-1BB, and OX-40. The CAR is in some cases a single polypeptide chain CAR. In some cases, the CAR comprises 2 polypeptide chains; for example, in some cases, the 2 polypeptide chains comprise dimerization domains that dimerize in the presence of a small molecule dimerizer.

A composition of the present disclosure can comprise, in addition to the synthetic immune cell and the second immune cell, a pharmaceutically acceptable excipient. Suitable excipient vehicles are, for example, water, saline, dextrose, glycerol, ethanol, or the like, and combinations thereof. In addition, if desired, the vehicle may contain minor amounts of auxiliary substances such as wetting or emulsifying agents or pH buffering agents. Actual methods of preparing such dosage forms are known, or will be apparent, to those skilled in the art. See, e.g., Remington's Pharmaceutical Sciences, Mack Publishing Company, Easton, Pa., 17th edition, 1985.

### Methods

The present disclosure provides a method of increasing proliferation and/or activity of a target immune cell in an individual. The method comprises administering to an individual in need thereof an effective amount (number) of a synthetic immune cell of the present disclosure, or an effective amount of a composition of the present disclosure (where the composition comprises a synthetic immune cell of the present disclosure and a second immune cell).

The target immune cell in the individual can be a tumor infiltrating lymphocyte (TIL), a cytotoxic T cell, a natural killer (NK) cell, or a regulatory T cell (Treg). The target immune cell in the individual can be one that is not genetically modified. The target immune cell in the individual can be one that is genetically modified, e.g., genetically modified to express an exogenous TCR, a CAR, or a BiTE. The target immune cell can be one that has been genetically modified to express a variant IL-2Rβ polypeptide on its cell surface. For example, the target immune cell can be one that has been genetically modified to express a variant IL-2Rβ polypeptide, as described above, where the variant IL-2Rβ binds an ortho-IL-2 polypeptide as described above.

In some cases, the target immune cell is specific for a cancer-associated antigen. In some cases, the target immune is specific for the same cancer-associated antigen to which the antibody present in the chimeric polypeptide binds. In some cases, the target immune cell is specific for a cancer-associated antigen; and the antibody present in the chimeric polypeptide is specific for a tissue-specific antigen. In some cases, the target immune cell is specific for a cancer-associated antigen; and the antibody present in the chimeric polypeptide is specific for a cell type-specific antigen.

In some cases, a method of the present disclosure comprises administering to an individual in need thereof a synthetic immune cell of the present disclosure, where from 10² to 10⁹ synthetic immune cells of the present disclosure are administered. For example, from 10² to about 10³, from about 10³ to about 10⁴, from about 10⁴ to about 10⁵, from about 10⁵ to about 10⁶, from about 10⁶ to about 10⁷, from about 10⁷ to about 10⁸, or from about 10⁸ to about 10^{9,} synthetic immune cells of the present disclosure are administered.

In some cases, a method of the present disclosure comprises administering to an individual in need thereof: a) a synthetic immune cell of the present disclosure; and b) a cytokine or proliferation-inducing polypeptide that increases proliferation and/or activity of a target effector immune cell. Suitable cytokines are described above. In some cases, the cytokine is a variant IL-2 polypeptide, as described above.

In some instances, a method of the instant disclosure finds use in treating a cancer. Cancers that can be treated using a method of the present disclosure include, but are not limited to, Acute Lymphoblastic Leukemia (ALL), Acute Myeloid Leukemia (AML), Adrenocortical Carcinoma, AIDS-Related Cancers (e.g., Kaposi Sarcoma, Lymphoma, etc.), Anal Cancer, Appendix Cancer, Astrocytomas, Atypical Teratoid/Rhabdoid Tumor, Basal Cell Carcinoma, Bile Duct Cancer (Extrahepatic), Bladder Cancer, Bone Cancer (e.g., Ewing Sarcoma, Osteosarcoma and Malignant Fibrous Histiocytoma, etc.), Brain Stem Glioma, Brain Tumors (e.g., Astrocytomas, Central Nervous System Embryonal Tumors, Central Nervous System Germ Cell Tumors, Craniopharyngioma, Ependymoma, etc.), Breast Cancer (e.g., female breast cancer, male breast cancer, childhood breast cancer, etc.), Bronchial Tumors, Burkitt Lymphoma, Carcinoid Tumor (e.g., Childhood, Gastrointestinal, etc.), Carcinoma of Unknown Primary, Cardiac (Heart) Tumors, Central Nervous System (e.g., Atypical Teratoid/Rhabdoid Tumor, Embryonal Tumors, Germ Cell Tumor, Lymphoma, etc.), Cervical Cancer, Childhood Cancers, Chordoma, Chronic Lymphocytic Leukemia (CLL), Chronic Myelogenous Leukemia (CML), Chronic Myeloproliferative Neoplasms, Colon Cancer, Colorectal Cancer, Craniopharyngioma, Cutaneous T-Cell Lymphoma, Duct (e.g., Bile Duct, Extrahepatic, etc.), Ductal Carcinoma In Situ (DCIS), Embryonal Tumors, Endometrial Cancer, Ependymoma, Esophageal Cancer, Esthesioneuroblastoma, Ewing Sarcoma, Extracranial Germ Cell Tumor, Extragonadal Germ Cell Tumor, Extrahepatic Bile Duct Cancer, Eye Cancer (e.g., Intraocular Melanoma, Retinoblastoma, etc.), Fibrous Histiocytoma of Bone (e.g., Malignant, Osteosarcoma, ect.), Gallbladder Cancer, Gastric (Stomach) Cancer, Gastrointestinal Carcinoid Tumor, Gastrointestinal Stromal Tumors (GIST), Germ Cell Tumor (e.g., Extracranial, Extragonadal, Ovarian, Testicular, etc.), Gestational Trophoblastic Disease, Glioma, Hairy Cell Leukemia, Head and Neck Cancer, Heart Cancer, Hepatocellular (Liver) Cancer, Histiocytosis (e.g., Langerhans Cell, etc.), Hodgkin Lymphoma, Hypopharyngeal Cancer, Intraocular Melanoma, Islet Cell Tumors (e.g., Pancreatic Neuroendocrine Tumors, etc.), Kaposi Sarcoma, Kidney Cancer (e.g., Renal Cell, Wilms Tumor, Childhood Kidney Tumors, etc.), Langerhans Cell Histiocytosis, Laryngeal Cancer, Leukemia (e.g., Acute Lymphoblastic (ALL), Acute Myeloid (AML), Chronic Lymphocytic (CLL), Chronic Myelogenous (CML), Hairy Cell, etc.), Lip and Oral Cavity Cancer, Liver Cancer (Primary), Lobular Carcinoma In Situ (LCIS), Lung Cancer (e.g., Non-Small Cell, Small Cell, etc.), Lymphoma (e.g., AIDS-Related, Burkitt, Cutaneous T-Cell, Hodgkin, Non-Hodgkin, Primary Central Nervous System (CNS), etc.), Macroglobulinemia (e.g., Waldenström, etc.), Male Breast Cancer, Malignant Fibrous Histiocytoma of Bone and Osteosarcoma, Melanoma, Merkel Cell Carcinoma, Mesothelioma, Metastatic Squamous Neck Cancer with Occult Primary, Midline Tract Carcinoma Involving NUT Gene, Mouth Cancer, Multiple Endocrine Neoplasia Syndromes, Multiple Myeloma/Plasma Cell Neoplasm, Mycosis Fungoides, Myelodysplastic Syndromes, Myelodysplastic/Myeloproliferative Neoplasms, Myelogenous Leukemia (e.g., Chronic (CML), etc.), Myeloid Leukemia (e.g., Acute (AML), etc.), Myeloproliferative Neoplasms (e.g., Chronic, etc.), Nasal Cavity and Paranasal Sinus Cancer, Nasopharyngeal Cancer, Neuroblastoma, Non-Hodgkin Lymphoma, Non-Small Cell Lung Cancer, Oral Cancer, Oral Cavity Cancer (e.g., Lip, etc.), Oropharyngeal Cancer, Osteosarcoma and Malignant Fibrous Histiocytoma of Bone, Ovarian Cancer (e.g., Epithelial, Germ Cell Tumor, Low Malignant Potential Tumor, etc.), Pancreatic Cancer, Pancreatic Neuroendocrine Tumors (Islet Cell Tumors), Papillomatosis, Paraganglioma, Paranasal Sinus and Nasal Cavity Cancer, Parathyroid Cancer, Penile Cancer, Pharyngeal Cancer, Pheochromocytoma, Pituitary Tumor, Pleuropulmonary Blastoma, Primary Central Nervous System (CNS) Lymphoma, Prostate Cancer, Rectal Cancer, Renal Cell (Kidney) Cancer, Renal Pelvis and Ureter, Transitional Cell Cancer, Retinoblastoma, Rhabdomyosarcoma, Salivary Gland Cancer, Sarcoma (e.g., Ewing, Kaposi, Osteosarcoma, Rhabdomyosarcoma, Soft Tissue, Uterine, etc.), Sezary Syndrome, Skin Cancer (e.g., Childhood, Melanoma, Merkel Cell Carcinoma, Nonmelanoma, etc.), Small Cell Lung Cancer, Small Intestine Cancer, Soft Tissue Sarcoma, Squamous Cell Carcinoma, Squamous Neck Cancer (e.g., with Occult Primary, Metastatic, etc.), Stomach (Gastric) Cancer, T-Cell Lymphoma, Testicular Cancer, Throat Cancer, Thymoma and Thymic Carcinoma, Thyroid Cancer, Transitional Cell Cancer of the Renal Pelvis and Ureter, Ureter and Renal Pelvis Cancer, Urethral Cancer, Uterine Cancer (e.g., Endometrial, etc.), Uterine Sarcoma, Vaginal Cancer, Vulvar Cancer, Waldenström Macroglobulinemia, Wilms Tumor, and the like. Thus, in some cases, an individual in need thereof is an individual who has a cancer, e.g., who has been diagnosed as having a cancer.

In some cases, a method of the present disclosure finds use in treating an autoimmune disorder. Thus, in some cases, an individual in need thereof is an individual who has been diagnosed as having an autoimmune disorder.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure numbered 1-55 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

Aspect 1. A genetically modified, in vitro immune cell, wherein the immune cell is genetically modified with one or more nucleic acids comprising nucleotide sequences encoding: a) a chimeric polypeptide comprising: i) an antibody specific for a target antigen; and ii) a binding triggered transcriptional activator; and b) a cytokine or proliferation-inducing polypeptide that increases proliferation and/or activity of an effector immune cell, wherein the nucleotide sequence encoding the cytokine or proliferation-inducing polypeptide is operably linked to a transcriptional control element responsive to the transcriptional activator.

Aspect 2. The genetically modified immune cell of aspect 1, wherein the immune cell is a T cell or a macrophage.

Aspect 3. The genetically modified immune cell of aspect 1, wherein the binding triggered transcriptional activator comprises, from N-terminal to C-terminal and in covalent linkage:
i) an extracellular domain comprising an antibody specific for a target antigen;
ii) a Notch regulatory polypeptide that comprises one or more proteolytic cleavage sites; and
iii) an intracellular domain comprising a transcriptional activator,

wherein binding of the antibody to the target antigen induces cleavage of the Notch receptor polypeptide at the one or more proteolytic cleavage sites, thereby releasing the intracellular domain.

Aspect 4. The genetically modified immune cell of aspect 3, wherein the Notch regulatory region comprises a Lin 12-Notch repeat, a heterodimerization domain comprising an S2 proteolytic cleavage site and a transmembrane domain comprising an S3 proteolytic cleavage site.

Aspect The genetically modified immune cell of aspect 3 or aspect 4, wherein the Notch regulatory region further comprises, at its N-terminus, one or more epidermal growth factor (EGF) repeats.

Aspect 6. The genetically modified immune cell of aspect 1, wherein the binding triggered transcriptional activator comprises, from N-terminal to C-terminal and in covalent linkage:
a) an extracellular domain comprising an antibody specific for a target antigen;
b) a non-Notch force sensor cleavage domain comprising a proteolytic cleavage site;
c) a cleavable transmembrane domain; and
d) an intracellular domain comprising a Notch intracellular signaling domain comprising a transcriptional activator, wherein binding of the antibody to the target antigen induces cleavage of the non-Notch force sensor cleavage domain at the proteolytic cleavage site, thereby releasing the intracellular domain, and wherein the non-Notch force sensor cleavage domain is selected from the group consisting of: a von Willebrand Factor (vWF) cleavage domain, an amyloid-beta cleavage domain, a CD16 cleavage domain, a CD44 cleavage domain, a Delta cleavage domain, a cadherin cleavage domain, an ephrin-type receptor or ephrin ligand cleavage domain, a protocadherin cleavage domain, a filamin cleavage domain, a synthetic E cadherin cleavage domain, an interleukin-1 receptor type 2 (IL1R2) cleavage domain, a major prion protein (PrP) cleavage domain, a neuregulin cleavage domain and an adhesion-GPCR cleavage domain,

Aspect 7. The genetically modified immune cell of aspect 6, wherein the non-Notch force sensor cleavage domain is a vWF cleavage domain.

Aspect 8. The genetically modified immune cell of aspect 7, wherein the vWF cleavage domain comprises a vWF A2 domain or a variant thereof.

Aspect 9. The genetically modified immune cell of any one of aspects 1-8, wherein the antibody is a nanobody, a diabody, a triabody, a minibody, a F(ab′)₂ fragment, a Fab fragment, a single chain variable fragment (scFv) or a single domain antibody (sdAb).

Aspect 10. The genetically modified immune cell of any one of aspects 1-9, wherein the cytokine is IL-2.

Aspect 11. The genetically modified immune cell of aspect 10, wherein the IL-2 is an IL-2 variant that exhibits increased binding affinity for IL-2R13 compared to wild-type IL-2.

Aspect 12. The genetically modified T cell of aspect 11, wherein the IL-2 variant comprises amino acid substitutions L80F, R81D, L85V, I86V, and I92F, compared to wild-type human IL-2.

Aspect 13. The genetically modified immune cell of aspect 10, wherein the IL-2 is a variant that preferentially activates regulatory T cells (T regs).

Aspect 14. The genetically modified immune cell of aspect 10, wherein the IL-2 is a variant that preferentially activates natural killer (NK) cells.

Aspect 15. The genetically modified immune cell of aspect 10, wherein the IL-2 is:
i) a variant IL-2 that binds to a variant IL-2Rβ comprising one or more amino acid substitutions selected from Q70Y, T73D, T73Y, H133D, H133E, H133K, Y134F, Y134E, and Y134R; and ii) exhibits reduced binding to wild-type IL-2Rβ.

Aspect 16. The genetically modified immune cell of aspect 15, wherein the variant IL-2 comprises one or more amino acid substitutions selected from: i) H16N, L19V, D20N, Q22T, M23H, and G27K; ii) E15D, H16N, L19V, D20L, Q22T, and M23H; iii) E15D, H16N, L19V, D20L, Q22T, and M23A; or iv) E15D, H16N, L19V, D20L, Q22K, M23A.

Aspect 17. The genetically modified immune cell of any one of aspects 1-9, wherein the cytokine is IL-15 or IL7.

Aspect 18. The genetically modified immune cell of any one of aspects 1-9, wherein the proliferation-inducing polypeptide binds IL-2Rβγ_{c} heterodimer, but does not bind IL-2Rα or IL-2Rβ.

Aspect 19. The genetically modified immune cell of any one of aspects 1-18, wherein the target antigen is a cancer-associated antigen.

Aspect 20. The genetically modified immune cell of aspect 19, wherein the cancer-associated antigen is selected from CD19, CD20, CD38, CD30, Her2/neu, ERBB2, CA125, MUC-1, prostate-specific membrane antigen (PSMA), CD44 surface adhesion molecule, mesothelin, carcinoembryonic antigen (CEA), epidermal growth factor receptor (EGFR), EGFRvIII, vascular endothelial growth factor receptor-2 (VEGFR2), high molecular weight-melanoma associated antigen (HMW-MAA), MAGE-A1, IL-13R-a2, and GD2.

Aspect 21. The genetically modified immune cell of any one of aspects 1-18, wherein the target antigen is tissue-specific antigen or an organ-specific antigen or a cell type-specific antigen.

Aspect 22. The genetically modified immune cell of any one of aspects 1-18, wherein the target antigen is a stromal cell antigen.

Aspect 23. The genetically modified immune cell of any one of aspects 1-22, wherein the genetically modified immune cell is a T cell that does not express an endogenous major histocompatibility complex (MHC) class I polypeptide on its surface.

Aspect 24. The genetically modified immune cell of aspect 23, wherein the genetically modified immune cell comprises a deletion of all or a portion of at least one MHC class I coding region.

Aspect 25. The genetically modified immune cell of aspect 24, wherein the at least one MHC class I coding region is a β2-microglobulin coding region.

Aspect 26. The genetically modified immune cell of any one of aspects 1-25, wherein the genetically modified immune cell is a T cell that does not express an endogenous T-cell receptor (TCR).

Aspect 27. A composition comprising:
a) the genetically modified immune cell of any one of aspects 1-26; and
b) a cytotoxic T cell (CTL).

Aspect 28. The composition of aspect 27, wherein the CTL is genetically modified to express:
a) an exogenous T-cell receptor (TCR), wherein the exogenous TCR is specific for a target antigen; or
b) a chimeric antigen receptor (CAR), wherein the CAR is specific for a target antigen; or
c) a bispecific T-cell engager (BiTE), wherein the BiTE comprises: i) a first antigen-binding region specific for CD3; and ii) a second antigen-binding region specific for a target antigen other than CD3.

Aspect 29. The composition of aspect 27 or aspect 28, wherein the TCR, the CAR, or the BiTE is specific for a cancer-associated antigen, and wherein the antibody present in the chimeric polypeptide is specific for a cancer-associated antigen.

Aspect 30. The composition of aspect 29, wherein the TCR, the CAR, or the BiTE is specific for the same cancer-associated antigen as the cancer-associate antigen to which the antibody present in the chimeric polypeptide binds.

Aspect 31. The composition of aspect 29, wherein the TCR, the CAR, or the BiTE is specific for a cancer-associated antigen that is different from the cancer-associate antigen to which the antibody present in the chimeric polypeptide binds.

Aspect 32. The composition of any one of aspects 28-31, wherein the CTL is genetically modified to express a CAR, and wherein the CAR comprises: a) an extracellular domain comprising the antigen-binding domain; b) a transmembrane region; and c) an intracellular signaling domain.

Aspect 33. The composition of aspect 32, wherein the intracellular signaling domain comprises: i) a signaling domain from the zeta chain of human CD3; and ii) one or more costimulatory polypeptides.

Aspect 34. The composition of aspect 33, wherein the one or more costimulatory polypeptides is selected from CD28, 4-1BB, and OX-40.

Aspect 35. The composition of any one of aspects 32-34, wherein the CAR is a single polypeptide chain.

Aspect 36. The composition of any one of aspects 32-34, wherein the CAR comprises 2 polypeptide chains.

Aspect 37. The composition of aspect 36, wherein the 2 polypeptide chains dimerize in the presence of a small molecule dimerizer.

Aspect 38. A method of increasing proliferation and/or activity of a target immune cell in an individual, the method comprising administering to the individual a genetically modified immune cell according to any one of aspects 1-26 or a composition according to any one of aspects 27-37.

Aspect 39. The method of any one of aspects 38-40, wherein the target immune cell is a tumor infiltrating lymphocyte (TIL), a cytotoxic T cell, a natural killer (NK) cell, or a regulatory T cell (Treg).

Aspect 40. The method of aspect 38 or aspect 39, wherein the target immune cell is an endogenous immune cell.

Aspect 41. The method of aspect 38 or aspect 39, wherein the target immune cell is an exogenous immune cell that has been genetically modified and introduced into the individual.

Aspect 42. The method of aspect 41, wherein the target immune cell is T cell that has been genetically modified to express an exogenous T-cell receptor (TCR) or an exogenous chimeric antigen receptor (CAR).

Aspect 43. The method of aspect 41 or aspect 42, wherein the target immune cell is genetically modified to express a variant IL2 receptor on its surface.

Aspect 44. The method of any one of aspects 38-43, wherein the target immune cell is specific for a cancer-associated antigen.

Aspect 45. The method of aspect 44, wherein the target immune cell is specific for the same cancer-associated antigen to which the antibody present in the chimeric polypeptide binds.

Aspect 46. The method of any one of aspects 38-45, comprising administering to the individual an effective amount of a cytokine or proliferation-inducing polypeptide that increases proliferation and/or activity of an effector immune cell.

Aspect 47. The method of aspect 46, wherein the cytokine is IL-2.

Aspect 48. The method of aspect 47, wherein the IL-2 is an IL-2 variant that exhibits increased binding affinity for IL-2R13 compared to wild-type IL-2.

Aspect 49. The method of aspect 48, wherein the IL-2 variant comprises amino acid substitutions L80F, R81D, L85V, I86V, and I92F, compared to wild-type human IL-2.

Aspect 50. The method of aspect 47, wherein the IL-2 is a variant that preferentially activates regulatory T cells (T regs).

Aspect 51. The method of aspect 47, wherein the IL-2 is a variant that preferentially activates natural killer (NK) cells.

Aspect 52. The method of aspect 47, wherein the IL-2 is: i) a variant IL-2 that binds to a variant IL-2Rβ comprising one or more amino acid substitutions selected from Q70Y, T73D, T73Y, H133D, H133E, H133K, Y134F, Y134E, and Y134R; and ii) exhibits reduced binding to wild-type IL-2Rβ.

Aspect 53. The method of aspect 52, wherein the variant IL-2 comprises one or more amino acid substitutions selected from: i) H16N, L19V, D2ON, Q22T, M23H, and G27K; ii) E15D, H16N, L19V, D20L, Q22T, and M23H; iii) E15D, H16N, L19V, D20L, Q22T, and M23A; or iv) E15D, H16N, L19V, D20L, Q22K, M23A.

Aspect 54. The method of aspect 46, wherein the cytokine is IL-15 or IL-7.

Aspect 55. The method of aspect 46, wherein the proliferation-inducing polypeptide binds IL-2Rβγ_{c} heterodimer, but does not bind IL-2Rα or IL-2Rβ.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1

Synthetic helper T cells were designed and generated. The synthetic helper T cells inducibly secrete proliferative cytokines upon recognition of a local tumor antigen. These synthetic helper cells expanded effector T cells or NK cells by secreting an enhanced-affinity interleukin (IL)-2. In a two-tumor mouse model, synthetic helper cells locally expanded T cells only in a tumor expressing the cognate triggering antigen, and potently enhanced tumor killing by T cells expressing a weak TCR.

### Materials and Methods

### Lentiviral DNA Constructs

SynNotch sequences such as “anti-CD19 synNotch” (anti-CD19-scFv-Notch-Gal4VP64) or “anti-GFP synNotch (LaG16-LaG2 tandem anti-GFP nanobody-Notch-Gal4VP64) were cloned downstream of a pGK or SFFV promoter, and response elements contained payloads (typically fluorescent protein-P2A-cytokine) were cloned downstream of a mCMV promoter and GAL4UAS, both in the pHR backbone, either a single multicistronic constructs or one for each component.

### T Cell Culture

T cells were isolated from Leukopaks using CD8+ selection kits, following which they were frozen in RPMI with 20% human AB serum and 10% DMSO. For assays, frozen T cells were thawed in T cell media (ImmunoCult) with IL-2 (always 30 U/mL unless otherwise specified) and resuspended at 1e6 cells/mL on day -13 (relative to the experiment start date) and activated with 25 uL anti-CD3/anti-CD28 beads per 1e6 T cells on day -12. Meanwhile, Lx293t lentiviral packaging cells were seeded in 6-well plates at 7e5 cells/well in DMEM F21 with 10% FBS on day -14, and transfected with pHR constructs for transfer into T cells as well as pCMV and pMD2.g packaging plasmids using FuGene on day -13. On day -11, viral supernatant was collected, cells removed by centrifugation at 500×g for 5 min, and applied to T cells at 1 mL per le7 T cells for single construct transductions or 0.75 mL of each construct for double construct transductions in 24-well plates. On day -10, T cells with virus were centrifuged at 400×g for 4 minutes, virus discarded, and T cells resuspended in media with IL-2. Cells were sorted for positive transduction on a BD FACSAria II or FACSAria Fusion on day -7. Cells were sorted for expression of a fluorescent protein marker or for positive staining of a Myctag on synNotch, or both.

Cells with synNotch were also negatively sorted on the fluorescent protein marker of synNotch activation in order to remove cells with high basal activity of synNotch (leaky cells). On days -5 through -3, T cells were counted with a Countess and Immunocult with IL-2 was added to dilute cell concentration to 5e5 cells/mL. On day 0, cells were used in assays in vitro or in vivo assays.

### Tumor Cell Culture

K562 cells were cultured in Iscove's Modified Dulbecco's Modified Eagle Medium with 10% FBS and split to 2.5e5 cells/mL every 3 days or 3.5e5 cells/mL every 2 days. A375 cells were cultured in DMEM with Glutamax and 10% FBS and split 1:6 every 2 days or 1:10 every 3 days.

### In Vitro Assay Preparation

T cells and target cells were washed of residual media and cytokines by two rounds of centrifugations at 400×g for 4 minutes followed by resuspension in Immunocult without IL-2. In some cases, T cells were stained with 1:5000 CellTrace proliferation stain (source) following manufacturer's protocol. After counting, various numbers of T cells and tumor cells were added to wells of 96 well plates, with or without various concentrations of IL-2, in 200 uL total volumes, with 2-3 replicates per conditions. For continuously-cultured experiments, at least 25 uL of each well was removed for analysis at regular time intervals (typically every 2-4 days) and replaced with fresh media. For closed-end experiments, multiple replicate time points were created on day 0 and analyzed destructively at different days (typically every 2-4 days).

### Mouse Experiments

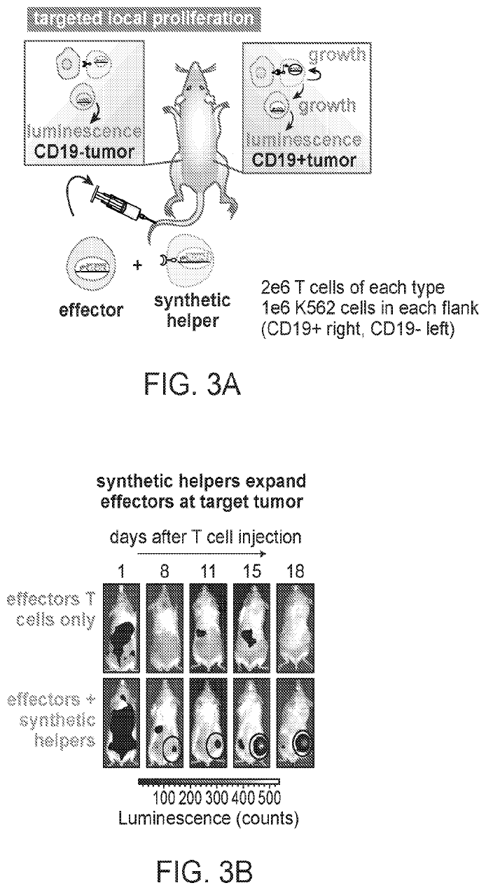

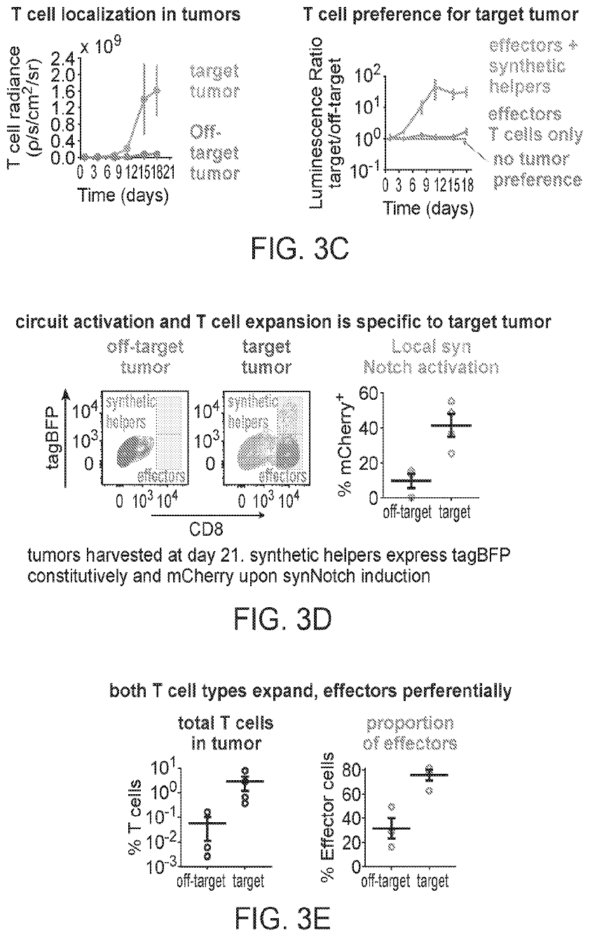

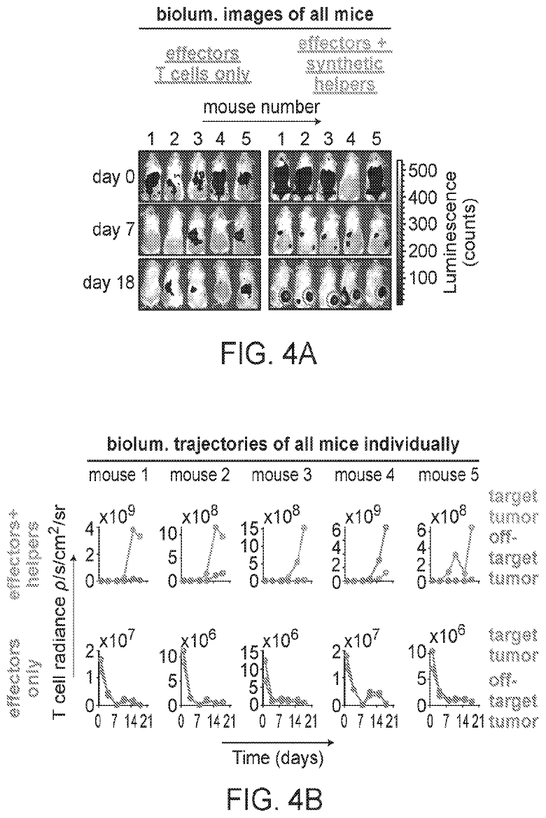

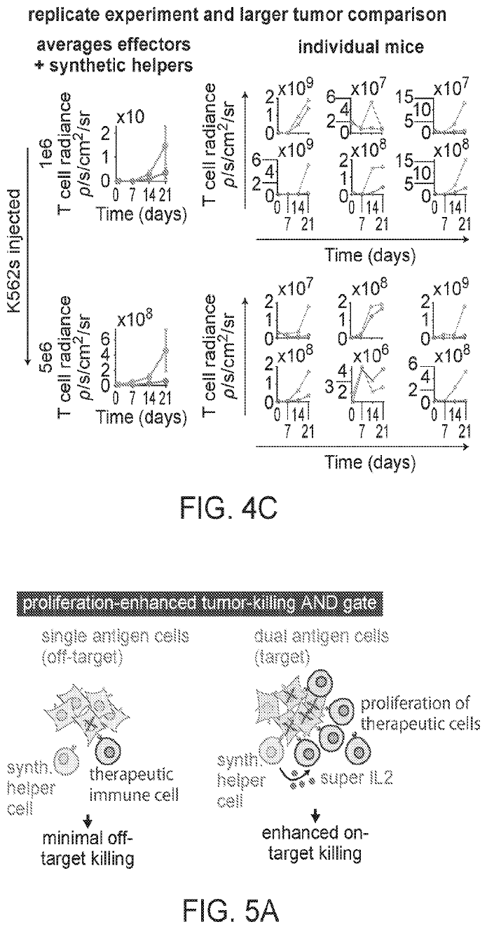

T cells were washed in phosphate-buffered saline (PBS), resuspended at 10 times the injection amount per mL, and 100 uL was injected into immunocompromised NOD scid gamma mice via the tail vein on day 0. Target tumor cells washed in PBS, resuspend at 10 times the injection amount per mL, and 100 uL was injected subcutaneously in the flank on day 0 (experiments in FIG. 3) or day -4 (experiments in FIG. 4). Tumor size was measured by caliper. Bioluminescence imaging was performed using an IVIS as follows: mice were injected intraperitoneally with 200 uL d-luciferin and imaged 15 minutes later. Excised tumors were cut in half and send for histology by HistoWiz or dissociated to perform flow cytometry. To prepare for flow cytometry, tumors were minced with razor blades, digested with collagenase and DNAse, passed through cell strainers, washed with PBS with EDTA, and stained. Excised spleens were prepared for flow cytometry by gently forcing through cell strainers with a rubber syringe plunger, then washing with PBS.

### Flow Cytometry and Analysis

Cells in 96 well plates were analyzed by flow cytometry on a BD LSRii or BD LSRFortessa X-20 with high-throughput system. For cell counting experiments, cells were not washed to ensure minimal errors due to cell loss: cells in media were mixed and transferred from the assay plates to new round bottom plates and PBS with Sytox Red, Blue, or Green was added to a final dilution of 1:500 in 200 uL total volume. For other experiments, plates were centrifuged at 400×g for 4 minutes, supernatant discarded, and stained with 1:500 Live/Dead Near Infrared in PBS. Plates were centrifuged, supernatant discarded, followed by staining with fluorescently labeled antibodies at various dilutions in PBS with 5% FBS. Wells were washed twice with and resuspended in PBS with 5% FBS. Compensation was performed using single antibody-stained Ultracomp beads, single antibody-stained transduced or untransduced cells, or unstained transduced cells expressing a single fluorescent protein. Analysis of flow data was performed in FlowJo. Cells were resolved from debris, anti-myctac beads, and bubbles based on forward scatter and side scatter signal. Cell singlets were isolated by the ratio of forward scatter area to height signal. Live cells were discriminated using Sytox or Live/Dead signal. Different cell types were distinguished based on the presence or absence unique fluorescent protein expression or antibody staining combinations.

### Results

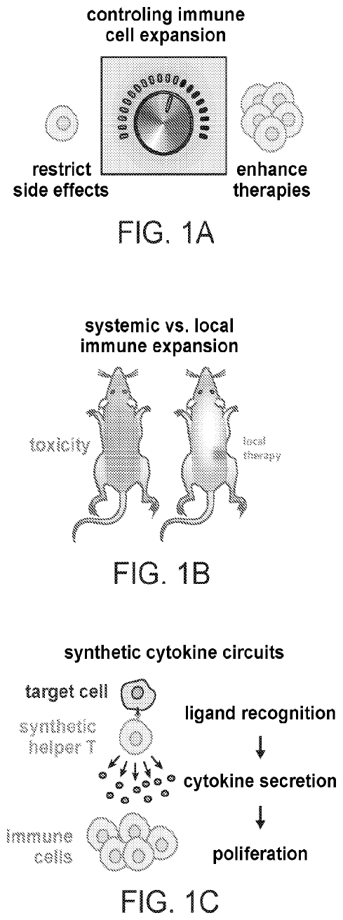

The results are shown in FIGS. 1-6.

FIG. 1A-1C: A. Controlling immune cell expansion is critical for cell therapies to balance between amplification and side effects. B. Local proliferation is important to enhance therapies at relevant tissues without causing systemic toxicity. C. A synthetic helper T cell could recognize target tissues or tumors and secrete cytokines to cause local proliferation of immune cells.

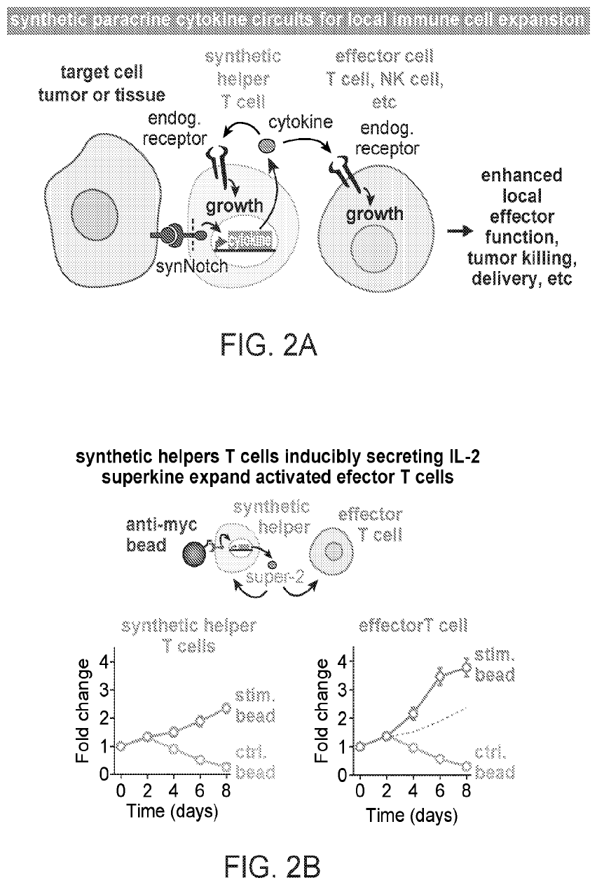

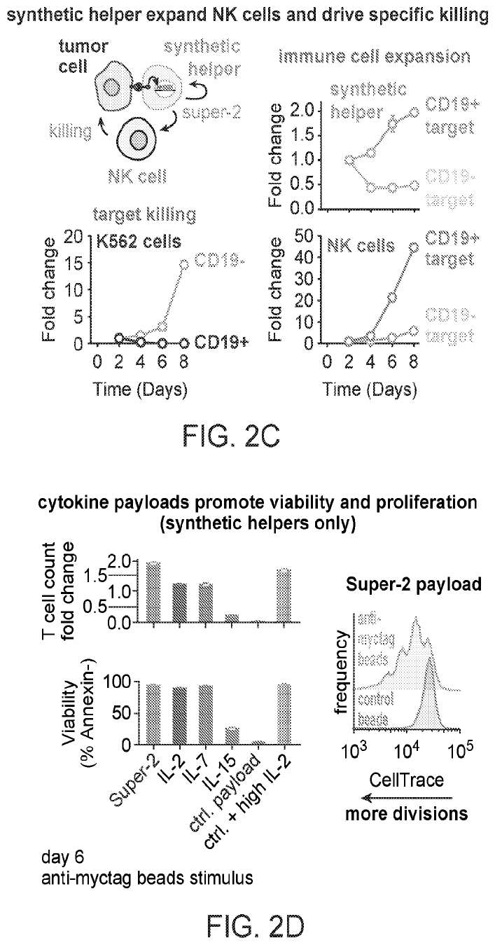

FIG. 2A-2C: Synthetic helper T cells with inducible cytokine circuits for immune cell expansion. A. Synthetic helper T cells use synNotch to secrete cytokines in response to target cell recognition, which are sensed via endogenous receptors, increasing survival, growth, and activation. B. Synthetic helper T cells were co-cultured with CD8+ effector T cells and stimulated with anti-myctag beads that trigger synNotch by binding the myc-tag on its N-terminus. Both synthetic helper T cells (left below diagram, blue) and effector T cells (right below diagram, orange) proliferated in response to stimulating beads but died in response to control beads (dark vs. light color). Effector cells proliferated more than synthetic helper cells (dashed blue line). CFSE dilution confirms fold change data (right of diagram): synthetic helper cells inducibly caused effector proliferation comparable to high doses of IL-2 (increasing from light gray to black: 0, 30, 100, 300 U/mL). C. Synthetic helper T cells were co-cultured with NK cells and K562 tumor cells expressing CD19 ligand (dark lines) or not (light lines). SynNotch binding of CD19 caused synthetic helper cells to secrete Super-2, which caused T cell and NK cell proliferation (right column, top and bottom respectively) and killing of K562 cells (lower left).

FIG. 3A-3E. Synthetic helper T cells can drive locally targeted proliferation in vivo. A. Setup of mouse experiment with bilateral tumors. Right (“target”) tumors express CD19 ligand whereas left (“off-target”) tumors do not. Synthetic helper T cells with anti-CD19 synNotch driving Super-2 were coinjected with effector T cells expressing eff-luc. B. Bioluminescence imaging of eff-luc expressing effector T cells with or without helpers over 18 days following T cell injection. Orange circles highlight proliferation in the target tumor. One characteristic mouse per group is shown. C. Quantification of bioluminescence signal in each tumor for mice receiving both effector and synthetic helper T cells showing specific effector T cell expansion in the target tumor. D. Ratio of luminescence signal in the target tumor to that in the off-target tumor, showing that effector cells with synthetic helper cells preferentially localize on the target side, whereas effector cells alone have no target/off-target localization preference. D. Tumors were harvested at day 21 from mice given both effector and synthetic helper cells and dissociated. Flow cytometry resolves CD8+ T cells from non-T cells and tagBFP+synthetic helper cells from effector cells. mCherry, a marker of synNotch activation, was more frequently expressed among synthetic helper cells in the target tumor than the off-target tumor. E. Percentage of T cells (among all live cell events in flow analysis) and percentage of effectors (among T cell events) show higher frequency in the target tumor relative to the off-target tumor.

FIG. 4A-4C. Bioluminescence images of individual mice over 18 days, from the same experiment shown in FIG. 3A-3E, showing accumulation of effluc+effector T cells in the target tumor (orange circle) only when synthetic helper T cells are coinjected (right group under blue line). Without synthetic helper T cells (left group under orange line), effector cells tend to localize to the spleen in small amounts but do not accumulate in either tumor. B. Time trajectories of total bioluminescence signal from effluc+effector T cell in either the target (blue or orange line) or off-target tumor (gray), quantified from the images in A, confirming specific accumulation of effector T cells in the target tumor when synthetic helper T cells are added (top row as compared to bottom). C. Replication of the experiment in FIG. 3A-3E with T cells from another donor (top left: group averages; top right: individual mice) as well as an additional run with larger tumors created by injecting 5e6 K562s rather of 1e6 (bottom left: group averages; bottom right: individual mice), both confirming the same trend on average.

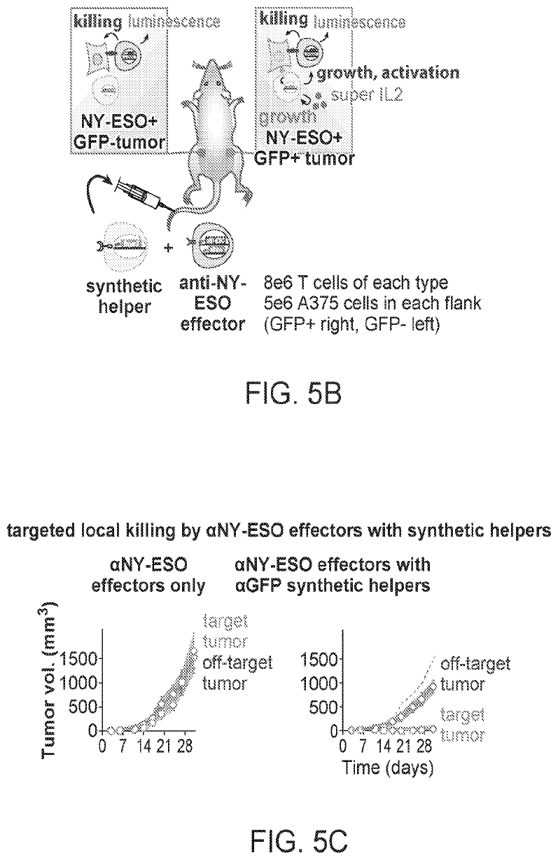

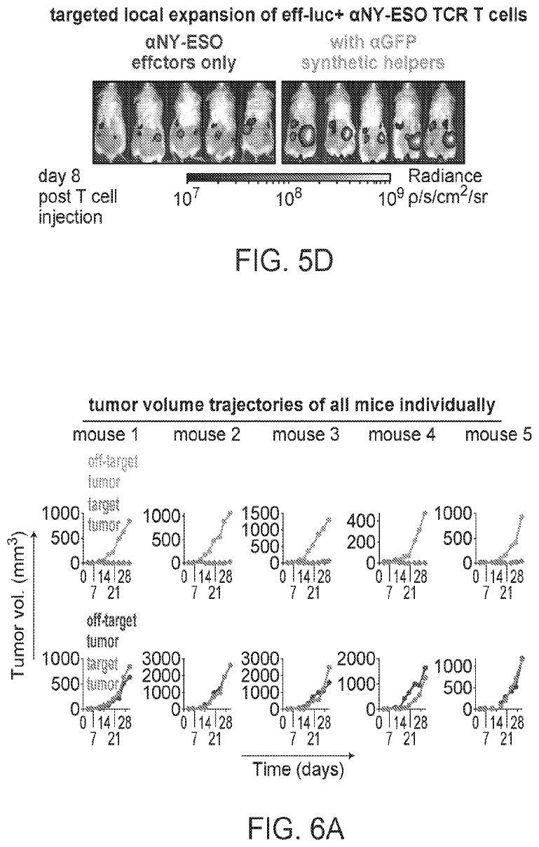

FIG. 5A-5D. A. Synthetic helpers form a functional AND-gate circuit with cytotoxic effectors to specifically enhance local killing of target tumors. B. In vivo experiment in NSG mice C. Tumor volume increases for effectors only but decreases in a target-specific way when synthetic helpers are added. D. Bioluminescence of effectors at an early time point (day 8) shows that synthetic helpers caused proliferation specifically in the target tumor.

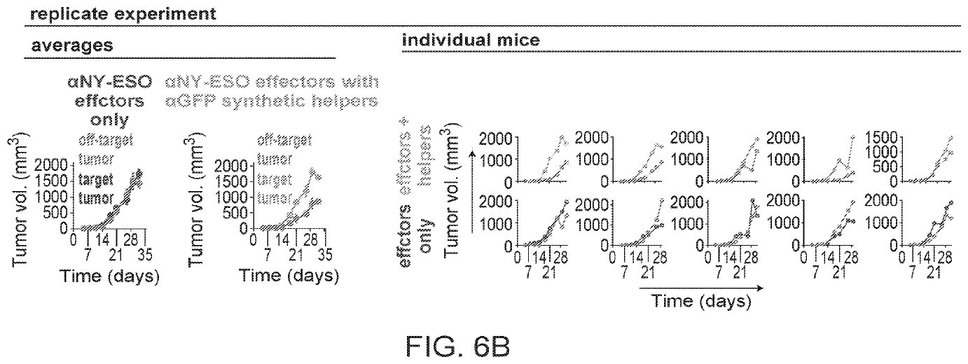

FIG. 6A-6B. Tumor volume trajectories of individual mice from the experiment shown in FIG. 5A-5D. Lower target tumor volume (top 5 plots, gray) vs. off-target (blue) demonstrates specific killing of the target tumor by anti-NY-ESO TCR+effector T cells only in the presence of coinjected synthetic helper T cells. Without synthetic helper T cells (bottom 5 plots), anti-NY-ESO TCR+effector T cells are ineffective at reducing the volume of either target (gray) or off-target (purple) tumors. B. Replication of the experiment shown in FIG. 5A-5D with T cells from another donor. Average tumor volume over time (left two plots) as well as tumor volume trajectories for individual mice (right 10 plots, following the layout of A) confirm the same trend.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A genetically modified, in vitro immune cell, wherein the immune cell is genetically modified with one or more nucleic acids comprising nucleotide sequences encoding:
a) a chimeric polypeptide comprising: i) an antibody specific for a target antigen; and ii) a binding triggered transcriptional activator; and
b) a cytokine or proliferation-inducing polypeptide that increases proliferation and/or activity of an effector immune cell,
wherein the nucleotide sequence encoding the cytokine or proliferation-inducing polypeptide is operably linked to a transcriptional control element responsive to the transcriptional activator.

2. The genetically modified immune cell of claim 1, wherein the immune cell is a T cell or a macrophage.

3. The genetically modified immune cell of claim 1, wherein the binding triggered transcriptional activator comprises, from N-terminal to C-terminal and in covalent linkage:
i) an extracellular domain comprising an antibody specific for a target antigen;
ii) a Notch regulatory polypeptide that comprises one or more proteolytic cleavage sites; and
iii) an intracellular domain comprising a transcriptional activator,
wherein binding of the antibody to the target antigen induces cleavage of the Notch receptor polypeptide at the one or more proteolytic cleavage sites, thereby releasing the intracellular domain

4. The genetically modified immune cell of claim 3, wherein the Notch regulatory region comprises a Lin 12-Notch repeat, a heterodimerization domain comprising an S2 proteolytic cleavage site and a transmembrane domain comprising an S3 proteolytic cleavage site.

5. The genetically modified immune cell of claim 3 or claim 4, wherein the Notch regulatory region further comprises, at its N-terminus, one or more epidermal growth factor (EGF) repeats.

6. The genetically modified immune cell of claim 1, wherein the binding triggered transcriptional activator comprises, from N-terminal to C-terminal and in covalent linkage:
a) an extracellular domain comprising an antibody specific for a target antigen;
b) a non-Notch force sensor cleavage domain comprising a proteolytic cleavage site;
c) a cleavable transmembrane domain; and
d) an intracellular domain comprising a Notch intracellular signaling domain comprising a transcriptional activator, wherein binding of the antibody to the target antigen induces cleavage of the non-Notch force sensor cleavage domain at the proteolytic cleavage site, thereby releasing the intracellular domain, and wherein the non-Notch force sensor cleavage domain is selected from the group consisting of: a von Willebrand Factor (vWF) cleavage domain, an amyloid-beta cleavage domain, a CD16 cleavage domain, a CD44 cleavage domain, a Delta cleavage domain, a cadherin cleavage domain, an ephrin-type receptor or ephrin ligand cleavage domain, a protocadherin cleavage domain, a filamin cleavage domain, a synthetic E cadherin cleavage domain, an interleukin-1 receptor type 2 (IL1R2) cleavage domain, a major prion protein (PrP) cleavage domain, a neuregulin cleavage domain and an adhesion-GPCR cleavage domain,

7. The genetically modified immune cell of claim 6, wherein the non-Notch force sensor cleavage domain is a vWF cleavage domain

8. The genetically modified immune cell of claim 7, wherein the vWF cleavage domain comprises a vWF A2 domain or a variant thereof.

9. The genetically modified immune cell of any one of claims 1-8, wherein the antibody is a nanobody, a diabody, a triabody, or a minibody, a F(ab′)₂ fragment, a Fab fragment, a single chain variable fragment (scFv) or a single domain antibody (sdAb).

10. The genetically modified immune cell of any one of claims 1-9, wherein the cytokine is IL-2.

11. The genetically modified immune cell of claim 10, wherein the IL-2 is an IL-2 variant that exhibits increased binding affinity for IL-2Rβ compared to wild-type IL-2.

12. The genetically modified T cell of claim 11, wherein the IL-2 variant comprises amino acid substitutions L80F, R81D, L85V, I86V, and I92F, compared to wild-type human IL-2.

13. The genetically modified immune cell of claim 10, wherein the IL-2 is a variant that preferentially activates regulatory T cells (T regs).

14. The genetically modified immune cell of claim 10, wherein the IL-2 is a variant that preferentially activates natural killer (NK) cells.

15. The genetically modified immune cell of claim 10, wherein the IL-2 is: i) a variant IL-2 that binds to a variant IL-2Rβ comprising one or more amino acid substitutions selected from Q70Y, T73D, T73Y, H133D, H133E, H133K, Y134F, Y134E, and Y134R; and ii) exhibits reduced binding to wild-type IL-2Rβ.

16. The genetically modified immune cell of claim 15, wherein the variant IL-2 comprises one or more amino acid substitutions selected from: i) H16N, L19V, D2ON, Q22T, M23H, and G27K; ii) E15D, H16N, L19V, D2OL, Q22T, and M23H; iii) E15D, H16N, L19V, D2OL, Q22T, and M23A; or iv) E15D, H16N, L19V, D2OL, Q22K, M23A.

17. The genetically modified immune cell of any one of claims 1-9, wherein the cytokine is IL-15 or IL-7.

18. The genetically modified immune cell of any one of claims 1-9, wherein the proliferation-inducing polypeptide binds IL-2Rβγ_{c} heterodimer, but does not bind IL-2Rα or IL-2Rβ.

19. The genetically modified immune cell of any one of claims 1-18, wherein the target antigen is a cancer-associated antigen.

20. The genetically modified immune cell of claim 19, wherein the cancer-associated antigen is selected from CD19, CD20, CD38, CD30, Her2/neu, ERBB2, CA125, MUC-1, prostate-specific membrane antigen (PSMA), CD44 surface adhesion molecule, mesothelin, carcinoembryonic antigen (CEA), epidermal growth factor receptor (EGFR), EGFRvIII, vascular endothelial growth factor receptor-2 (VEGFR2), high molecular weight-melanoma associated antigen (HMW-MAA), MAGE-A1, IL-13R-a2, and GD2.

21. The genetically modified immune cell of any one of claims 1-18, wherein the target antigen is tissue-specific antigen or an organ-specific antigen or a cell type-specific antigen.

22. The genetically modified immune cell of any one of claims 1-18, wherein the target antigen is a stromal cell antigen.

23. The genetically modified immune cell of any one of claims 1-22, wherein the genetically modified immune cell is a T cell that does not express an endogenous major histocompatibility complex (MHC) class I polypeptide on its surface.

24. The genetically modified immune cell of claim 23, wherein the genetically modified immune cell comprises a deletion of all or a portion of at least one MHC class I coding region.

25. The genetically modified immune cell of claim 24, wherein the at least one MHC class I coding region is a β2-microglobulin coding region.

26. The genetically modified immune cell of any one of claims 1-25, wherein the genetically modified immune cell is a T cell that does not express an endogenous T-cell receptor (TCR).

27. A composition comprising:
a) the genetically modified immune cell of any one of claims 1-26; and
b) a cytotoxic T cell (CTL).

28. The composition of claim 27, wherein the CTL is genetically modified to express:
a) an exogenous T-cell receptor (TCR), wherein the exogenous TCR is specific for a target antigen; or
b) a chimeric antigen receptor (CAR), wherein the CAR is specific for a target antigen; or
c) a bispecific T-cell engager (BiTE), wherein the BiTE comprises: i) a first antigen-binding region specific for CD3; and ii) a second antigen-binding region specific for a target antigen other than CD3.

29. The composition of claim 27 or claim 28, wherein the TCR, the CAR, or the BiTE is specific for a cancer-associated antigen, and wherein the antibody present in the chimeric polypeptide is specific for a cancer-associated antigen.

30. The composition of claim 29, wherein the TCR, the CAR, or the BiTE is specific for the same cancer-associated antigen as the cancer-associate antigen to which the antibody present in the chimeric polypeptide binds.

31. The composition of claim 29, wherein the TCR, the CAR, or the BiTE is specific for a cancer-associated antigen that is different from the cancer-associate antigen to which the antibody present in the chimeric polypeptide binds.

32. The composition of any one of claims 28-31, wherein the CTL is genetically modified to express a CAR, and wherein the CAR comprises: a) an extracellular domain comprising the antigen-binding domain; b) a transmembrane region; and c) an intracellular signaling domain

33. The composition of claim 32, wherein the intracellular signaling domain comprises: i) a signaling domain from the zeta chain of human CD3; and ii) one or more costimulatory polypeptides.

34. The composition of claim 33, wherein the one or more costimulatory polypeptides is selected from CD28, 4-1BB, and OX-40.

35. The composition of any one of claims 32-34, wherein the CAR is a single polypeptide chain.

36. The composition of any one of claims 32-34, wherein the CAR comprises 2 polypeptide chains.

37. The composition of claim 36, wherein the 2 polypeptide chains dimerize in the presence of a small molecule dimerizer.

38. A method of increasing proliferation and/or activity of a target immune cell in an individual, the method comprising administering to the individual a genetically modified immune cell according to any one of claims 1-26 or a composition according to any one of claims 27-37.

39. The method of claim 38, wherein the target immune cell is a tumor infiltrating lymphocyte (TIL), a cytotoxic T cell, a natural killer (NK) cell, or a regulatory T cell (Treg).

40. The method of claim 38 or claim 39, wherein the target immune cell is an endogenous immune cell.

41. The method of claim 38 or claim 39, wherein the target immune cell is an exogenous immune cell that has been genetically modified and introduced into the individual.

42. The method of claim 41, wherein the target immune cell is T cell that has been genetically modified to express an exogenous T-cell receptor (TCR) or an exogenous chimeric antigen receptor (CAR).

43. The method of claim 41 or claim 42, wherein the target immune cell is genetically modified to express a variant IL2 receptor on its surface.

44. The method of any one of claims 38-43, wherein the target immune cell is specific for a cancer-associated antigen.

45. The method of claim 44, wherein the target immune cell is specific for the same cancer-associated antigen to which the antibody present in the chimeric polypeptide binds.

46. The method of any one of claims 38-45, comprising administering to the individual an effective amount of a cytokine or proliferation-inducing polypeptide that increases proliferation and/or activity of an effector immune cell.

47. The method of claim 46, wherein the cytokine is IL-2.

48. The method of claim 47, wherein the IL-2 is an IL-2 variant that exhibits increased binding affinity for IL-2R13 compared to wild-type IL-2.

49. The method of claim 48, wherein the IL-2 variant comprises amino acid substitutions L80F, R81D, L85V, I86V, and I92F, compared to wild-type human IL-2.

50. The method of claim 47, wherein the IL-2 is a variant that preferentially activates regulatory T cells (T regs).

51. The method of claim 47, wherein the IL-2 is a variant that preferentially activates natural killer (NK) cells.

52. The method of claim 47, wherein the IL-2 is: i) a variant IL-2 that binds to a variant IL-2Rβ comprising one or more amino acid substitutions selected from Q70Y, T73D, T73Y, H133D, H133E, H133K, Y134F, Y134E, and Y134R; and ii) exhibits reduced binding to wild-type IL-2Rβ.

53. The method of claim 52, wherein the variant IL-2 comprises one or more amino acid substitutions selected from: i) H16N, L19V, D2ON, Q22T, M23H, and G27K; ii) E15D, H16N, L19V, D20L, Q22T, and M23H; iii) E15D, H16N, L19V, D20L, Q22T, and M23A; or iv) E15D, H16N, L19V, D20L, Q22K, M23A.

54. The method of claim 46, wherein the cytokine is IL-15 or IL-7.

55. The method of claim 46, wherein the proliferation-inducing polypeptide binds IL-2Rβγ_{c} heterodimer, but does not bind IL-2Rα or IL-2Rβ.
